(19) United States
(12) Patent Application Publication
(10) Pub. No.: US 2023/0024904 A1
(43) Pub. Date: Jan. 26, 2023

(54) DELIVERY COMPOSITIONS AND METHODS

(71) Applicant: EDITY THERAPEUTICS LTD., Ness Ziona (IL)

(72) Inventors: Michal GOLAN MASHIACH, Ness Ziona (IL); Yoav MANASTER, Tel Aviv (IL)

(21) Appl. No.: 17/855,014

(22) Filed: Jun. 30, 2022

Related U.S. Application Data
(63) Continuation of application No. PCT/IL2021/050007, filed on Jan. 3, 2021.
(60) Provisional application No. 62/956,342, filed on Jan. 2, 2020.

(51) Int. Cl. A61K 35/15

(52) U.S. Cl. CPC A61K 35/15

(57) ABSTRACT

Modified cells comprising an exogenous non-cytotoxic therapeutic proteinaceous agent are provided. Non-cytotoxic chimeric polypeptides comprising a lymphocyte lytic granule-secreted protein or a functional fragment thereof and a protein of interest are also provided. Therapeutic compositions, nucleic acid molecules and methods of use related to the modified lymphocytes and chimeric polypeptides of the invention are also provided.

### CROSS REFERENCE TO RELATED APPLICATIONS

This application is a Bypass Continuation of International Application No. PCT/IL2021/050007 having International filing date of Jan. 3, 2021, which claims the benefit of priority of U.S. Provisional Patent Application No. 62/956,342, filed Jan. 2, 2020, the contents of which are all incorporated herein by reference in their entirety.

### FIELD OF INVENTION

The present invention is in the field of cell-based delivery systems.

### BACKGROUND OF THE INVENTION

There is an urgent need for biological delivery systems that can introduce therapeutic agents into the cytoplasm or even the nucleus of target cells. While delivery systems that can bring therapeutic agents to the cell surface of target cells exist, they are only effective if their target is a cell surface receptor, or if the therapeutic agent contains a cell-penetrating moiety. Thus, many otherwise-druggable cytoplasmic targets are not currently amenable to treatment, due to the lack of delivery systems that can reach them.

In recent years, genome-editing technologies have been developed to exhibit high efficiency, specificity and versatility. Nevertheless, the abovementioned limited ability to effectively deliver treatment into cells equally applies to these novel technologies.

The hurdles that impede effective application of gene therapy technologies include immunogenicity, the need for specificity to the target organ, tissue or cell and the lack of adequate composition, packaging, functional characteristics, and stability of delivery vehicles. Current gene therapy and gene-editing delivery systems for systemic gene therapy, whether viral or non-viral, are rapidly cleared from the circulation following systemic administration. In addition, the immunogenicity of viral systems can elicit an acute inflammatory response, which at best limits the effect of subsequent doses and at worst can be hazardous to the patient. Therefore, there is still a need for efficient and precise gene editing delivery systems.

Leukocytes, and in particular lymphocytes, have been widely investigated and utilized as cytotoxic therapeutic agents. These cells have been perfected by hundreds of millions of years of evolution to surveil, target and potentially kill, when necessary, any cell in the body. Lymphocyte-induced killing is a highly efficient and highly specific process, which occurs with minimal collateral damage, sparing neighboring bystander cells. Several lineages of lymphocytes, such as T cells, NK cells and NKT cells, can directly adhere to target cells and secrete the content of their lytic granules into those cells, and thus subsequently kill them.

Two key proteins are involved in this process—Perforin assembles on the target cell membrane and forms pores, through which Granzyme, the lytic protein, enters the target cell and initiates an apoptotic cascade. Although, delivery of Granzyme has been envisioned as method of killing cancer cells, it has never been used as a delivery/non-cytotoxic option. International Patent Application WO2015/157864, as well as articles Dalken et al., “Targeted induction of apoptosis by chimeric granzyme B fusion proteins carrying antibody and growth factor domains for cell recognition”, Oberoi et al., 2013 “EGFR-targeted granzyme B expressed in NK cells enhances natural cytotoxicity and mediates specific killing of tumor cells”, and Zhoa et al., “Secreted antibody/granzyme B fusion protein stimulates selected killing of HER2-overexpressing tumor cells” all make use of cytotoxic cells and cytotoxic granzyme fusions proteins to enhance immune cell killing of target cells. In all of these cases the goal is the death of the target cell, and in all cases the cells natural cytotoxicity is enhanced. A non-cytotoxic method of protein transfer, that highjacks lymphocytes' natural lytic pathway to deliver therapeutic agents, in particular gene therapies, would provide a formidable solution to the therapeutic delivery problem. Converting this cell killing pathway to a delivery pathway provides a novel solution that heretofore was not envisioned.

### SUMMARY OF THE INVENTION

The present invention provides modified cells comprising an exogenous non-lytic therapeutic proteinaceous agent. Non-lytic chimeric polypeptides comprising a lymphocyte lytic granule-secreted protein, or a functional fragment thereof, and a protein of interest are also provided. Therapeutic compositions comprising the modified cells, nucleic acid molecules encoding the chimeric polypeptides and methods of use are also provided.

According to a first aspect, there is provided a modified lymphocyte or myeloid cell, wherein the lymphocyte or myeloid cell comprises an exogenous non-cytotoxic therapeutic proteinaceous agent.

According to some embodiments, the lymphocyte or myeloid cell does not comprise the non-cytotoxic therapeutic proteinaceous agent within the lymphocyte or myeloid cell's cellular membrane.

According to some embodiments, the agent comprises a therapeutic portion that is not any of:
a. a naturally secreted protein,
b. a membranal protein expressed in the membrane of the modified cell,
c. a surface receptor-binding agent,
d. a viral penetration or envelope protein, and
e. a nanoparticle conjugated or encapsulated agent.

According to some embodiments, the lymphocyte is selected from a T cell and a natural killer (NK) cell, or the myeloid cell is a macrophage.

According to some embodiments, the lymphocyte is a non-cytotoxic lymphocyte.

According to some embodiments, the lymphocyte or myeloid cell is a cell of a cell line or a primary cell.

According to some embodiments, the therapeutic proteinaceous agent is in a secretory granule of the lymphocyte.

According to some embodiments, the therapeutic portion is a cytoplasmic or nuclear protein.

According to some embodiments, the therapeutic proteinaceous agent does not comprise a signal peptide.

According to some embodiments, the therapeutic proteinaceous agent is an RNA-protein complex.

According to some embodiments, the therapeutic proteinaceous agent comprises a molecular weight of at least 50 kDa.

According to some embodiments, the therapeutic proteinaceous agent comprises a chimeric protein comprising a lymphocyte lytic granule-secreted protein or a functional fragment thereof or variant thereof and a therapeutic polypeptide.

According to some embodiments, the lymphocyte lytic granule-secreted protein is directly conjugated to the therapeutic polypeptide by a peptide bond or is indirectly conjugated by a protein linker.

According to some embodiments, the linker is a cleavable linker.

According to some embodiments, the cleavable linker is cleaved in acidic pH.

According to some embodiments, the therapeutic polypeptide comprises a nuclear localization sequence (NLS).

According to some embodiments, the lymphocyte lytic granule-secreted protein is selected from the group consisting of: granzyme A, granzyme B, granzyme H, granzyme K, granzyme M, Granulysin, Serglycin, and Perforin.

According to some embodiments, the lymphocyte lytic granule-secreted protein is granzyme B.

According to some embodiments, the lymphocyte lytic granule-secreted protein is non-cytotoxic or inactivated.

According to some embodiments, the therapeutic proteinaceous agent is a genome-editing agent.

According to some embodiments, the genome-editing agent comprises CRISPR associated protein 9 (Cas9).

According to some embodiments, the genome-editing agent comprises a meganuclease.

According to another aspect, there is provided a therapeutic composition comprising a modified lymphocyte of the invention.

According to some embodiments, the therapeutic composition of the invention is formulated for administration to a subject, and comprises a pharmaceutically acceptable carrier, excipient, or adjuvant or both.

According to another aspect, there is provided a non-cytotoxic chimeric polypeptide comprising a lymphocyte lytic granule-secreted protein or a functional fragment thereof and a protein of interest.

According to some embodiments, the protein of interest does not bind a cell surface receptor in the target cell.

According to some embodiments, the lymphocyte lytic granule-secreted protein is directly conjugated to the protein of interest by a peptide bond or is indirectly conjugated by a protein linker.

According to some embodiments, the linker is a cleavable linker, optionally wherein cleavable linker is cleaved in the secretory granule.

According to some embodiments, the protein of interest comprises at least one NLS.

According to some embodiments, the lymphocyte lytic granule-secreted protein is selected from the group consisting of: granzyme A, granzyme B, granzyme H, granzyme K, granzyme M, Granulysin, Serglycin, and Perforin.

According to some embodiments, the lymphocyte lytic granule-secreted protein is granzyme B.

According to some embodiments, the protein of interest is a genome-editing agent.

According to some embodiments, the genome-editing agent is CRISPR associated protein 9 (Cas9).

According to some embodiments, the genome-editing agent is a meganuclease.

According to another aspect, there is provided a polynucleotide encoding a chimeric polypeptide of the invention.

According to some embodiments, the polynucleotide is an expression vector capable of expressing the chimeric polypeptide in a lymphocyte or myeloid cell.

According to another aspect, there is provided a method of delivering a non-lytic therapeutic proteinaceous agent to a target cell, the method comprising contacting the target cell with any one of:
a. a modified lymphocyte or myeloid cell of the invention;
b. a therapeutic composition of the invention;
c. a modified lymphocyte or myeloid cell expressing a chimeric polypeptide of the invention; and
d. a modified lymphocyte or myeloid cell expressing a polynucleotide of the invention;

thereby delivering a non-cytotoxic therapeutic proteinaceous agent to a target cell.

According to some embodiments, the target cell is in a subject in need of treatment with the non-cytotoxic therapeutic proteinaceous agent.

According to some embodiments, the modified lymphocyte or myeloid cell is autologous or allogeneic to the subject.

According to some embodiments, the method of the invention comprises extracting lymphocytes or myeloid cells from the subject, expressing in the lymphocytes or myeloid cells a non-cytotoxic therapeutic proteinaceous agent to produce modified lymphocytes or myeloid cells and returning the modified lymphocytes or myeloid cells to the subject.

By another aspect, there is provided, the modified lymphocytes or myeloid cells of the invention or the pharmaceutical compositions of the invention for use in treating a subject in need thereof.

According to another aspect, there is provided a method of delivering a non-lytic therapeutic protein of interest into a target cell, the method comprising contacting the target cell with a modified leukocyte wherein the modified leukocyte comprises reduced cytotoxicity as compared to a non-modified leukocyte and comprises the non-lytic therapeutic protein of interest, thereby delivering a non-lytic therapeutic protein of interest into a target cell.

According to another aspect, there is provided a method of delivering a genome editing protein into a target cell, the method comprising contacting the target cell with a modified leukocyte wherein the modified leukocyte comprises the genome editing protein, thereby delivering a non-lytic therapeutic protein of interest into a target cell.

According to some embodiments, the non-lytic therapeutic protein of interest or genome editing protein is delivered to the cytoplasm or nucleus of the target cell.

According to some embodiments, the modified leukocyte is capable of forming an immune synapse with the target cell.

According to some embodiments, the modified leukocyte comprises the non-lytic therapeutic protein of interest or genome editing protein within a secretory lysosome.

According to some embodiments, the modified leukocyte does not comprise the non-lytic therapeutic protein of interest or genome editing protein within or conjugated to the modified cell's cellular membrane.

According to some embodiments, the modified leukocyte is selected from a modified T cell, modified natural killer (NK) cell and a modified myeloid cell.

According to some embodiments, the modified leukocyte is a modified non-cytotoxic leukocyte, or wherein the modified leukocyte has been further modified to reduce cytotoxicity.

According to some embodiments, the modified leukocyte comprises a knockout or knockdown or at least one endogenous cytotoxic protein, optionally wherein the endogenous cytotoxic protein is Granzyme B.

According to some embodiments, the modified leukocyte comprises a mutation in at least one endogenous cytotoxic protein and wherein the mutation decreases cytotoxicity of the endogenous cytotoxic protein.

According to some embodiments, the modified leukocyte comprises an anti-sense mediated reduction of at least one endogenous cytotoxic protein and wherein the anti-sense mediated decreases cytotoxicity of the endogenous cytotoxic protein.

According to some embodiments, the non-lytic therapeutic protein of interest does not comprise any of:
a. a naturally secreted protein;
b. a membranal protein expressed in the membrane of the modified leukocyte;
c. a surface receptor-binding protein;
d. a viral penetration or envelope protein; and
e. a nanoparticle conjugated or encapsulated protein.

According to some embodiments, the non-lytic therapeutic protein of interest or genome editing protein is a cytoplasmic or nuclear protein.

According to some embodiments, the non-lytic therapeutic protein of interest or genome editing protein does not comprise a signal peptide.

According to some embodiments, the non-lytic therapeutic protein of interest or genome editing protein is a ribonuclear-protein (RNP) complex.

According to some embodiments, the non-lytic therapeutic protein of interest comprises a genome editing protein.

According to some embodiments, the genome editing protein modifies a gene within a nucleus of the target cell.

According to some embodiments, the genome editing protein is a meganuclease.

According to some embodiments, the non-lytic therapeutic protein of interest or genome editing protein comprises a molecule weight of at least 50 kDa.

According to some embodiments, the genome editing protein is CRISPR associated protein 9 (Cas9).

According to some embodiments, the non-lytic therapeutic protein of interest is a chimeric protein comprising a lymphocyte lytic granule-secreted protein or a functional fragment or variant thereof and a therapeutic polypeptide or wherein the genome editing protein is a chimeric protein comprising a lymphocyte lytic granule-secreted protein or a functional fragment or variant thereof and the genome editing protein.

According to some embodiments, the lymphocyte lytic granule-secreted protein or a functional fragment or variant thereof comprises a signal peptide, optionally wherein the signal peptide is an N-terminal signal peptide.

According to some embodiments, the lymphocyte lytic granule-secreted protein is directly conjugated to the therapeutic polypeptide or genome editing protein by a peptide bond or is indirectly conjugated by a protein linker.

According to some embodiments, the linker is a cleavable linker, optionally wherein the linker is cleaved in a secretory granule or at acidic pH.

According to some embodiments, the therapeutic polypeptide or genome editing protein comprises a nuclear localization sequence (NLS).

According to some embodiments, the lytic granule-secreted protein is a lytic protein comprising at least one inactivating mutation, wherein the inactivating mutation inhibits the lytic function of the lytic protein.

According to some embodiments, the lymphocyte lytic granule secreted protein is selected from the group consisting of: granzyme A, granzyme B, granzyme H, granzyme K, granzyme M, Granulysin, Serglycin, and Perforin.

According to some embodiments, the lymphocyte lytic granule secreted protein is Granzyme B.

According to some embodiments, the transfer is not mediated by exosomes.

According to some embodiments, delivering to a cytoplasm does not comprise entering an endosome.

According to some embodiments, the method further comprises providing the leukocyte, activating the leukocyte and expressing the non-lytic therapeutic protein of interest or genome editing protein in the leukocyte after the activating to produce the modified leukocyte.

According to some embodiments, the expression is done not more than 5 days before the contacting.

According to some embodiments, the target cell is in a subject in need of treatment with the non-lytic therapeutic protein of interest or genome editing protein, and the method comprises administering a pharmaceutical composition comprising the modified leukocyte.

According to some embodiments, the subject is in need to gene therapy and the modified leukocyte comprises a genome editing protein.

According to some embodiments, the modified leukocyte is autologous or allogeneic to the subject.

According to some embodiments, the method comprises extracting leukocytes from the subject, activating the leukocytes, expressing the non-cytotoxic therapeutic protein of interest or genome editing protein in the leukocytes after the activating to produce the modified leukocytes and returning the modified leukocytes to the subject.

According to some embodiments, the expressing is done not more than 5 days before the returning.

According to some embodiments, the treatment does not comprise killing the target cell.

According to some embodiments, the subject does not suffer from cancer.

According to another aspect, there is provided a non-lytic chimeric polypeptide comprising a lymphocyte lytic granule-secreted protein or a functional fragment or variant thereof and a protein of interest.

According to some embodiments, the protein of interest does not bind a cell surface receptor.

According to some embodiments, the lymphocyte lytic granule-secreted protein is directly conjugated to the protein of interest by a peptide bond or is indirectly conjugated by a protein linker.

According to some embodiments, the linker is a cleavable linker, optionally wherein cleavable linker is cleaved in a secretory granule or at acidic pH.

According to some embodiments, the protein of interest comprises at least one NLS.

According to some embodiments, the lymphocyte lytic granule-secreted protein is selected from the group consisting of: granzyme A, granzyme B, granzyme H, granzyme K, granzyme M, and Perforin.

According to some embodiments, the lymphocyte lytic granule-secreted protein is granzyme B.

According to some embodiments, the protein of interest is a genome-editing protein.

According to some embodiments, the genome-editing protein is CRISPR associated protein 9 (Cas9).

According to some embodiments, the genome-editing protein is a meganuclease.

According to some embodiments, the protein of interest comprises a molecular weight of at least 50 kDa.

According to another aspect, there is provided a polynucleotide encoding a chimeric polypeptide of the invention.

According to some embodiments, the polynucleotide is an expression vector capable of expressing the chimeric polypeptide in a lymphocyte or myeloid cell.

According to another aspect, there is provided a modified leukocyte with reduced cytotoxicity as compared to a non-modified leukocyte, comprising at least one of:
a. a non-cytotoxic chimeric polypeptide of the invention;
b. a polynucleotide of the invention; and
c. a secretory granule comprising a non-cytotoxic therapeutic protein of interest.

According to some embodiments, the leukocyte is capable of forming an immune synapse with a target cell.

According to some embodiments, the leukocyte is selected from a T cell, a natural killer (NK) cell, and a myeloid cell.

According to some embodiments, the modified leukocyte does not comprise the non-cytotoxic therapeutic protein of interest within or conjugated to the modified leukocyte's cellular membrane.

According to some embodiments, the modified leukocyte is a modified non-cytotoxic leukocyte, or wherein the modified leukocyte comprises a mutation of at least one endogenous cytotoxic protein wherein the mutation decreases the cytotoxicity of the endogenous cytotoxic protein.

According to some embodiments, the modified cell comprises a knockout or knockdown or at least one endogenous cytotoxic protein, optionally wherein the endogenous cytotoxic protein is Granzyme B.

According to another aspect, there is provided a therapeutic composition comprising a modified leukocyte of the invention.

According to some embodiments, the composition is formulated for administration to a subject, and comprising a pharmaceutically acceptable carrier, excipient, or adjuvant or both.

According to another aspect, there is provided a kit comprising at least one of:
a. a non-cytotoxic chimeric polypeptide of the invention;
b. a polynucleotide of the invention;
c. a modified leukocyte of the invention; and
d. a therapeutic composition of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Schematic representation of fusion-protein expressing plasmids of the invention.

FIGS. 2A-E: Granzyme-Cherry protein transfer to K562 target cells. (2A) Dot plot of size separation of target K562 cells from effector T cells using FSC/SSC gating of total live events. (2B) Histogram of gating of GFP positive target K562 cells after co-culture with Granzyme-Cherry transduced T cells. (2C) Histogram of Cherry fluorescence in GFP positive target K562 cells (GFP+ gate as shown in 2B) after co-culture with either mock electroporated T cells (filled gray histogram) or T cells electroporated with mRNA IVT product of the coding region of plasmid 1_3 described in FIG. 1 encoding Granzyme-Cherry (empty black histogram). (2D) Histogram of Cherry fluorescence in GFP positive target K562 cells after co-cultured with either mock electroporated YTS cells (filled gray histogram) or YTS cells electroporated with mRNA IVT product of the coding region of plasmid 1_3 shown in FIG. 1 encoding Granzyme-Cherry (empty black histogram). (2E) Histogram of Cherry fluorescence in Tag-it labeled K562 cells after co-culture with Cherry expressing GZMB-KO YTS cells (used as negative control).

FIGS. 3A-D: Granzyme knock-out cells with functionality. (3A) Histogram of GZMB expression in parental (grey) and GZMB-KO (black line) YTS cells. (3B) Histogram of the size, as measured by forward-scatter of K562 cells co-cultured with YTS cells expressing endogenous GZMB (light grey) and K562 cells co-cultured with YTS GZMB-KO cells (dark grey). (3C) Histograms of mCherry expression in K562 cells co-cultured with YTS cells expressing endogenous GZMB (left histogram) mock electroporated (shaded) or electroporated with GZMB-crmCherry plasmid (grey outline) and with YTS GZMB-KO cells (right histogram) mock electroporated (shaded) or electroporated with GZMB-crmCherry plasmid (black outline). (3D) Histogram presentation of live/dead staining of K562 cells alone or after co-cultured with parental YTS cells or GZMB KO YTS.

FIGS. 4A-D: (4A) Representative micrographs for primary T cells electroporated with pMAX_hGZMB_HA-Cas9_P2A_crmCherry plasmid. Bright-field (BF), mCherry, Cas-9, Granzyme B (GZMB) and a merge of all the channels is shown. (4B) Histogram of gates used to define Tag-it labeled target K562 cells and unlabeled Tag-it negative effector YTS cells. (4C) Photograph of western blot detection of Cas9 in YTS cells over-expressing mRNA IVT product of hGZMB_Cas9 (first lane) and in K562 target cells co-cultured with the GZMB-CAS9 expressing YTS cells (second lane). YTS cells and K562 cells were sorted by Tag-it expression using the gate shown in 4B. The gray arrow indicates the Cas9 band in the K562 cells. A ladder is provided for size comparison and shows that the lower of the two bands observed in the YTS cells corresponds to the molecular weight of Cas9. (4D) Histogram of GZMB-Cas9 as detected by intra-cellular staining of Granzyme B in K562 target cells following co-culture with GZMB-KO YTS cells expressing GZMB-CAS9 or mock treated GZMB KO YTS cell.

FIGS. 5A-D: Editing in target cells after Granzyme mediated CAS9 and Meganuclease transfer. (5A-B) Micrographs of melanoma cells after co-culture with (5A) YTS cells and (5B) T cells electroporated with GZMB_Cas9 mRNA. RFP cells are shown (black arrow) and GFP positive cells indicate successful genome editing (white arrow). (5C) Histogram of GFP expressing cells in the RFP positive population of K562 target cells following transfer of GZMB-CAS9 from YTS cells electroporated with GZMB-CAS9 mRNA. (5D) Histogram of GFP expressing cells in the RFP positive population of K562 target cells following transfer of GZMB-Meganuclease from YTS cells electroporated with GZMB-Meganuclease mRNA.

FIG. 6A-C: K562 myeloid cell line functions as an effector cell capable of cargo delivery. (6A) Histogram of mCherry signal in Tag-it positive melanoma target cells after co-culture with K562 cells electroporated with GZMB-crmCherry mRNA IVT product of the coding region of plasmid 1_3 shown in FIG. 1. (6B) Histogram presentation of GFP signal in Tag-it positive melanoma target cells after co-culture with K562 cells electroporated with GZMB-Meganuclease-GFP mRNA IVT product of the coding region of plasmid 26 shown in FIG. 1. (6C) Histogram of GFP expressing cells in the RFP positive population of melanoma target cells following transfer of GZMB-CAS9 from K562 cells electroporated with GZMB-CAS9 mRNA.

FIGS. 7A-D: Cas9-GFP RNP transfer from T cells to P815 target cells. (7A-C) Histograms of (7A) GFP expression in Mock and Cas9-GFP RNA-Protein complex electroporated T cells, (7B) gates used for CD8 positive and negative differentiation between target P815 cells and CD8 positive T cells in co-culture experiments with Mock or Cas9-GFP transduced T cells, and (7C) GFP fluorescence in CD8 negative target P815 cells (CD8-gate as shown in 7B) co-cultured with either mock electroporated T cells or Cas9-GFP transduced T cells. Mock is shown as filled gray histograms and Cas9-GFP RNA-Protein complex transduced T cells shown as empty black histograms. (7D) Representative micrograph of co-cultured Cas9-GFP RNA-Protein complex transduced T cells and P815 cells. Cas9-GFP can be seen as green. CD8 positive T cells are shown as light blue. Cells that are only green and not blue are target cells that have received the RNA-Protein complex, some are marked by arrows.

FIGS. 8A-I: Cas9-GFP RNP transfer from YTS cells to K562 and MCF7 target cells. Histograms of (8A) Cas9-GFP fluorescence in mock electroporated and Cas9-GFP RNA-protein complex transduced YTS cells, (8B) gates used to define Tag-it labeled target K562 cells and unlabeled Tag-it negative effector YTS cells, (8C) Cas9-GFP RNA-Protein complex derived fluorescence in Tag-it positive target K562 cells (Tagit+ gate as shown in 8B) co-cultured with either mock electroporated YTS cells or Cas9-GFP transduced YTS cells, (8D) gates used to define Tag-it labeled target MCF7 cells and unlabeled Tag-it negative effector YTS cells, and (8E) Cas9-GFP RNA-Protein complex derived fluorescence in Tag-it positive target MCF7 cells (Tagit+ gate as shown in 8D) co-cultured with either mock electroporated YTS cells or Cas9-GFP transduced YTS cells. Mock electroporation experiments are shown as filled gray histograms and Cas9-GFP RNA-Protein complex electroporation experiments are shown as empty black histograms. (8F) Histograms of GFP signal in K562 cells incubated with the supernatant of the co-culture between Cas9-GFP transduced YTS cells and K562 cells. (8G) GFP signal of melanoma cells incubated with the supernatant of the co-culture between Cas9-GFP transduced YTS cells and melanoma cells. (8H) Cas9-GFP RNA-Protein complex derived fluorescence in Tag-it positive target K562 cells co-cultured with either mock electroporated YTS cells or Cas9-GFP transduced YTS cells (supernatant of this co-culture was used in FIG. 8F). (8I) Cas9-GFP RNA-Protein complex derived fluorescence in Tag-it positive target melanoma cells co-cultured with either mock electroporated YTS cells or Cas9-GFP transduced YTS cells (supernatant of this co-culture was used in FIG. 8G).

FIGS. 9A-C: Editing in target cells after CAS9 RNP transfer. (9A-C) Micrographs of melanoma cells after co-culture with (9A) YTS cells transduced with CAS9 RNPs, (9B) YTS Granzyme B Knockout cells transduced with CAS9 RNPs and (9C) K562 myeloid cells transduced with CAS9 RNPs. RFP cells are shown (black arrow) and GFP positive cells indicate successful genome editing (white arrow).

### DETAILED DESCRIPTION OF THE INVENTION

The present invention, in some embodiments, provides modified cells comprising an exogenous therapeutic agent. Chimeric polypeptides comprising a protein of interest and a lymphocyte lytic granule-secreted protein, a variant thereof or a fragment thereof, are also provided. Therapeutic compositions comprising the modified cells, nucleic acid molecules encoding the chimeric polypeptide and methods of use of the modified cells, pharmaceutical compositions and chimeric polypeptides of the invention are also provided.

The instant invention is based on the surprising finding that leukocytes, and in particular lymphocytes such as T cells and natural killer (NK) cells, can be used as delivery systems for therapeutic agents. In particular, it has been found that, though these cells are naturally cytotoxic, their cytotoxic function is not required for their delivery capability, and indeed non-cytotoxic delivery is superior to cytotoxic delivery when the desired outcome is not death of the target cell. It was first found that a fusion of a lytic granule-secreted protein, such as Granzyme, to a protein of interest can target that protein of interest to lytic granules and cause it to be secreted and even be taken up by target cells and reach the cell cytoplasm. This transfer was demonstrated even for very large proteins such as Cas-9, which is well over 100 kDa in size. The cytotoxic properties of granzyme are not required for this transfer, as a mutant granzyme and a pro-granzyme containing an inhibitory di-peptide were both equally capable of facilitating the protein transfer. Even more surprising, it was found that an RNA-protein complex, also of a very large size, when electroporated into lymphocytes or myeloid cells, was also transferred to target cells even without fusion to a lytic granule-secreted protein fusion. The complex was able to reach the interior of the target cells and indeed was functional there, as the RNA-Protein complex was capable of editing the target cell's genome. This heretofore unknown mechanism allows for lymphocytes to be used as a broad therapeutic delivery system. In particular, it provides for a delivery system for genome editing in target cells.

It will be understood by a skilled artisan that the leukocytes (lymphocytes and myeloid cells) of the invention are a mode of delivery for bringing the therapeutic agent to its target in a subject. Immune cells are well known in the art to home to disease locations in the body. As such, they are an ideal delivery method for carrying a therapeutic agent to sites of disease. A major stumbling block for current therapeutics is the fact that many known targets are cell internal, while reliable methods of intracellular delivery do not currently exist. As such, most therapeutics are limited to targeting cell surface molecules. This severely limits the pool of available druggable targets. In particular, gene therapy, which is potentially able to treat a wide variety of diseases/conditions, is only feasible if the gene-editing agents can be delivered into the nucleus. Additionally, short circulation half-life and biodistribution problems hamper many promising therapeutics. The cells and compositions of the invention offer a comprehensive solution to all these problems. The therapeutic agent is protected within the leukocyte while it travels through the body, thus limiting degradation and half-life concerns. The leukocyte reduces off-target effects, first by homing to sites of disease, either through its natural homing ability or thanks to targeting moieties on its cell surface. Off-target effects are further limited, as lymphocytes do not secrete lytic vesicles until the cell is activated, a mechanism that can be controlled and targeted directly to disease/target cells. Finally, the lytic vesicles are taken up by the target cells and their cargo is delivered into the target cell's interior, allowing for access to all cellular targets and even for genome editing. The combination of all these aspects of the present technology makes it uniquely suited as a therapeutic delivery system.

Chimeric Molecules

By a first aspect, there is provided a chimeric molecule comprising a lymphocyte lytic granule-secreted protein, or a variant or fragment thereof, and a molecule of interest.

It will be understood by a skilled artisan that the lymphocyte lytic granule-secreted protein, or a variant or fragment thereof, is a targeting moiety in the chimeric molecule, and the protein of interest is the cargo. The targeting moiety is not intended to possess any direct therapeutic function, but rather to facilitate transfer to target cells. Facilitating transfer comprises delivering the cargo to the lytic granule that is secreted by lymphocytes and taken up by target cells. The protein of interest is a therapeutic cargo, that is able to act on its target following delivery by the lymphocyte lytic granule-secreted protein.

In some embodiments, the chimeric molecule is a chimeric polypeptide. In some embodiments, the molecule of interest is a protein of interest. As used herein, the terms “peptide”, “polypeptide” and “protein” are used interchangeably to refer to a polymer of amino acid residues. In another embodiment, the terms “peptide”, “polypeptide” and “protein”, as used herein, encompass native peptides, peptidomimetics (typically including non-peptide bonds or other synthetic modifications) and the peptide analogs peptoids and semipeptoids or any combination thereof. In another embodiment, the peptides, polypeptides and proteins described carry modifications rendering them more stable while in the body, or more capable of penetrating into cells. In one embodiment, the terms “peptide”, “polypeptide” and “protein” apply to naturally-occurring amino acid polymers. In another embodiment, the terms “peptide”, “polypeptide” and “protein” apply to amino acid polymers in which one or more amino acid residue is an artificial chemical analog of a corresponding naturally-occurring amino acid.

In some embodiments, the chimeric polypeptide is a fusion protein. In some embodiments, the chimeric polypeptide is an artificial polypeptide. In some embodiments, the chimeric polypeptide is a polypeptide not found in nature. As used herein, the term “chimeric polypeptide” refers to a single polypeptide chain comprising at least two distinct protein domains or regions, that are not naturally found in the same protein. The fusion protein may be formed by the joining of two or more peptides through a peptide bond formed between the amino-terminus of one peptide and the carboxyl-terminus of another peptide. The fusion protein may be expressed as a single polypeptide fusion protein from a nucleic acid sequence encoding the single contiguous conjugate. In some embodiments, fusion proteins are created through the joining of two or more genes that originally coded for separate proteins. Recombinant fusion proteins may be created artificially by recombinant DNA technology for use in biological research or therapeutics. “Chimeric” or “chimera” usually designate hybrid proteins made of polypeptides having different functions or physicochemical patterns. For example, a fusion protein can comprise a first part that is a lymphocyte lytic-granule-secreted protein, and a second part (e.g., genetically fused to the first part) that is a protein of interest (e.g., a protein with a distinct enzymatic activity, i.e., DNA nuclease). Methods of fusion protein generation, recombinant protein generation, recombinant DNA generation, and DNA fusion techniques are well known in the art, and any such method for making the chimeric molecules of the invention may be employed. The chimeric polypeptide may be cleavable, such that upon cleavage it is separated into two or more polypeptides. In one non-limiting example, upon cleavage, a fusion polypeptide comprising a lymphocyte lytic-granule-secreted protein and a protein of interest is split into the lymphocyte lytic-granule-secreted protein and the protein of interest, such that the functionality of the protein of interest is intact.

As used herein, the term “recombinant protein” refers to a protein which is coded for by a recombinant nucleic acid molecule (DNA or RNA) and is thus not naturally occurring. The term “recombinant DNA or RNA” refers to DNA or RNA molecules formed by laboratory methods of genetic recombination. Generally, this recombinant molecule is in the form of an mRNA, a vector, a plasmid or a virus, used to express the recombinant protein in a cell.

In some embodiments, the lymphocyte lytic granule-secreted protein is directly conjugated to the protein of interest. In some embodiments, the lymphocyte lytic granule-secreted protein is linked to the protein of interest by a linker. In some embodiments, there are no intervening amino acids between the lymphocyte lytic granule-secreted protein and the protein of interest. In some embodiments, there is an amino acid linker separating the lymphocyte lytic granule-secreted protein and the protein of interest. In some embodiments, the lymphocyte lytic granule-secreted protein is N-terminal to the molecule of interest. In some embodiments, the lymphocyte lytic granule-secreted protein is C-terminal to the molecule of interest.

As used herein, the term “lymphocyte lytic granule-secreted protein” and “secretory lysosomal protein” are herein used interchangeably and refer to any protein that is secreted by the lymphocyte via lytic granules which are known as secretory lysosomes. In some embodiments, the lymphocyte lytic granule-secreted protein is a protein involved in the granzyme/perforin pathway and is naturally secreted from lytic granules of lymphocytes into target cells during antigen-dependent lymphocyte recognition of target cells. In some embodiments, antigen-dependent lymphocyte recognition is mediated by an endogenous T Cell Receptor (TCR). In some embodiments, antigen-dependent lymphocyte recognition is mediated by engineered T cell Receptor ligation to an MHC/peptide complex on a target cell. In some embodiments, antigen dependent lymphocyte recognition is mediated by Chimeric Antigen Receptor (CAR) ligation to an antigen on a target cell. In some embodiments, the secretory lysosomal protein is a lytic granule secreted protein. In some embodiments, the lytic granule secreted-protein is a secretory lysosomal protein.

In some embodiments, the lytic granule is a lytic vesicle. In some embodiments, the lytic granule is a secretory lysosome. The lytic granule is a well-known organelle found in lymphocytes. It is a specialized secretory organelle that, upon activation of cytotoxic lymphocytes such as T cells and NK cells, navigates via microtubules to the apical side of the cell, which is towards the lymphocyte-target cell synapse. The protein content of this granule is disclosed in the art, and can be investigated by isolating these granules by methods known in the art. In some embodiments, the lymphocyte lytic granule-secreted protein is any protein secreted from a lytic granule. In some embodiments, the lymphocyte lytic granule-secreted protein is any protein in a lytic granule. Proteins found in lytic granules are well known in the art and can be found for example in “Supramolecular attack particles are autonomous killing entities released from cytotoxic T cells”, Balint et al., Science, 2020 May 22; 368(6493):897-901, herein incorporated by reference in its entirety. In some embodiments, the lymphocyte lytic granule-secreted protein is provided in Table 1. In some embodiments, the lymphocyte lytic granule-secreted protein is selected from the proteins provided in Table 1. In some embodiments, the lymphocyte lytic granule-secreted protein is selected from the group consisting of: granzyme A, granzyme B, granzyme H, granzyme K, granzyme M, Granulysin, Serglycin, and Perforin. In some embodiments, the lymphocyte lytic granule-secreted protein is selected from the group consisting of: granzyme A, granzyme B, granzyme H, granzyme K, granzyme M, and Perforin. In some embodiments, the lymphocyte lytic granule-secreted protein is a granzyme. In some embodiments, a granzyme is selected from granzyme A, granzyme B, granzyme H, granzyme K, and granzyme M. In some embodiments, a granzyme is granzyme B. In some embodiments, the lymphocyte lytic granule-secreted protein is perforin. In some embodiments, the lymphocyte lytic granule-secreted protein is a human protein. In some embodiments, the lymphocyte lytic granule-secreted protein is a mammalian protein.

TABLE 1
Lytic granule protein list

| Gene name | Accession | Gene name | Accession |
|---|---|---|---|
| Keratin, type II cytoskeletal 1 | P04264 | T-complex protein 1 subunit | P17987 |
| | | alpha TCP1 | |
| Keratin, type I cytoskeletal 9 | P35527 | Skin-specific protein 32 XP32 | Q5T750 |
| Serum albumin ALB | P02768 | Mucin-like protein 1 MUCL1 | Q96DR8 |
| Keratin, type I cytoskeletal 10 | P13645; | 60S ribosomal protein L23a | P62750 |
| | Q7Z3Y7; | RPL23A | |
| | Q2M2I5; | | |
| | Q7Z3Y8; | | |
| | Q7Z3Z0; | | |
| | Q99456; | | |
| | O76013; | | |
| | Q7Z3Y9 | | |
| Keratin, type II cytoskeletal 2 | P35908 | Fatty acid synthase FASN | P49327 |
| epidermal 2 | | | |
| Complement C3 | P01024 | Phospholipase B-like 1 | Q6P4A8 |
| | | PLBD1 | |
| Hemoglobin subunit alpha | P69905; | Acid ceramidase ASAH1 | Q13510 |
| HBA1 | P02008 | | |
| Desmoplakin DSP | P15924 | T-complex protein 1 subunit | P48643 |
| | | epsilon CCT5 | |
| Desmoglein-1 DSG1 | Q02413 | Serine/threonine-protein | Q8IYT8 |
| | | kinase ULK2 ULK2 | |
| Keratin, type II cytoskeletal 5 | P13647 | Isocitrate dehydrogenase | P48735 |
| | | [NADP], mitochondrial IDH2 | |
| Alpha-enolase ENO1 | P06733; | Lupus La protein SSB | P05455 |
| | P13929 | | |
| Apolipoprotein B-100 APOB | P04114 | Spectrin alpha chain, non- | Q13813 |
| | | erythrocytic 1 SPTAN1 | |
| Keratin, type I cytoskeletal 16 | P08779; | Elongation factor Tu, | P49411 |
| | P35900 | mitochondrial TUFM | |
| Hornerin HRNR | Q86YZ3 | Macrophage migration | P14174 |
| | | inhibitory factor MIF | |
| Vimentin VIM | P08670; | Nucleosome assembly protein | P55209 |
| | P17661; | 1-like 1 NAP1L1 | |
| | P41219; | | |
| | P07196 | | |
| Pyruvate kinase PKM PKM | P14618; | D-3-phosphoglycerate | O43175 |
| | P30613 | dehydrogenase PHGDH | |
| Histone H4 HIST1H4A | P62805 | Inorganic pyrophosphatase | Q15181 |
| | | PPA1 | |
| Immunoglobulin mu heavy | P0DOX6 | Carboxypeptidase A4 CPA4 | Q9UI42 |
| chain | | | |
| Glyceraldehyde-3-phosphate | P04406; | Zymogen granule protein 16 | Q96DA0 |
| dehydrogenase GAPDH | O14556 | homolog B ZG16B | |
| Junction plakoglobin JUP | P14923; | Protein CREG1 CREG1 | O75629 |
| | P35222 | | |
| Inter-alpha-trypsin inhibitor | P19823 | Histidine ammonia-lyase HAL | P42357 |
| heavy chain H2 ITIH2 | | | |
| Alpha-2-macroglobulin A2M | P01023 | Loricrin LOR | P23490 |
| Actin, cytoplasmic 1 ACTB | P60709; | Programmed cell death protein | O14737 |
| | Q6S8J3; | 5 PDCD5 | |
| | A5A3E0; | | |
| | P0CG38; | | |
| | P0CG39; | | |
| | Q9BYX7 | | |
| Keratin, type II cytoskeletal 78 | Q8N1N4 | Calmodulin-like protein 5 | Q9NZT1 |
| | | CALML5 | |
| 60 kDa heat shock protein, | P10809 | 60S ribosomal protein L15 | P61313 |
| mitochondrial HSPD1 | | RPL15 | |
| Keratin, type I cytoskeletal 14 | P02533 | Polyadenylate-binding protein | Q4VXU2 |
| | | 1-like PABPC1L | |
| Annexin A2 ANXA2 | P07355; | Prohibitin-2 PHB2 | Q99623 |
| | A6NMY6 | | |
| Peptidyl-prolyl cis-trans | P62937; | Ezrin EZR | P15311; |
| isomerase A PPIA | Q9Y536; | | Q5TZA2 |
| | A0A075B759 | | |
| Putative elongation factor 1- | Q5VTE0; | Extracellular matrix protein 1 | Q16610 |
| alpha-like 3 EEF1A1P5 | Q05639 | ECM1 | |
| Phosphoglycerate kinase 1 | P00558; | Cystatin-A CSTA | P01040 |
| PGK1 | P07205 | | |
| Complement C4-A C4A | P0C0L4; | Protein S100-A16 S100A16 | Q96FQ6 |
| | P0C0L5 | | |
| Dermcidin DCD | P81605 | 60S ribosomal protein L31 | P62899 |
| | | RPL31 | |
| Immunoglobulin kappa | P01834; | Heterogeneous nuclear | Q1KMD3 |
| constant IGKC | P0DOX7 | ribonucleoprotein U-like | |
| | | protein 2 HNRNPUL2 | |
| Alpha-2-HS-glycoprotein | P02765 | 60S ribosomal protein L6 | Q02878 |
| AHSG | | RPL6 | |
| Histone H1.4 HIST1H1E | P10412; | Nascent polypeptide- | E9PAV3; |
| | P16402; | associated complex subunit | Q9BZK3 |
| | Q02539; | alpha, muscle-specific form | |
| | P22492 | NACA | |
| Inter-alpha-trypsin inhibitor | Q06033 | Proteasome subunit beta type- | P49720 |
| heavy chain H3 ITIH3 | | 3 PSMB3 | |
| Keratin, type I cytoskeletal 17 | Q04695 | Gamma- | O75223 |
| | | glutamylcyclotransferase | |
| | | GGCT | |
| Apolipoprotein A-I APOA1 | P02647 | Transaldolase TALDO1 | P37837 |
| Gelsolin GSN | P06396 | Protein POF1B POF1B | Q8WVV4 |
| Profilin-1 PFN1 | P07737 | Protein S100-A6 S100A6 | P06703 |
| Lipoprotein lipase LPL | P06858; | Hepatocyte growth factor | P14210 |
| | Q9Y5X9 | HGF | |
| Peroxiredoxin-1 PRDX1 | Q06830; | Plasma protease C1 inhibitor | P05155 |
| | Q13162 | SERPING1 | |
| Plastin-2 LCP1 | P13796; | Small nuclear | P62314 |
| | P13797 | ribonucleoprotein Sm D1 | |
| | | SNRPD1 | |
| Filaggrin-2 FLG2 | Q5D862 | 40S ribosomal protein S24 | P62847 |
| | | RPS24 | |
| Prolactin-inducible protein PIP | P12273 | C-C motif chemokine 28 | Q9NRJ3 |
| | | CCL28 | |
| Fructose-bisphosphate aldolase | P04075; | F-actin-capping protein | P47756 |
| A ALDOA | P05062 | subunit beta CAPZB | |
| L-lactate dehydrogenase A | P00338; | Stress-induced- | P31948 |
| chain LDHA | P07864 | phosphoprotein 1 STIP1 | |
| Desmocollin-1 DSC1 | Q08554 | Chymotrypsin-like elastase | P08861 |
| | | family member 3B CELA3B | |
| Elongation factor 2 EEF2 | P13639 | Keratin, type II cytoskeletal 7 | P08729 |
| | | 7 | |
| Fibrillin-1 FBN1 | P35555 | Transmembrane protein 109 | Q9BVC6 |
| | | TMEM109 | |
| Fibronectin FN1 | P02751 | Collagen alpha-2(IV) chain | P08572 |
| | | COL4A2 | |
| Keratin, type II cytoskeletal 1b | Q7Z794 | 60S ribosomal protein L7a | P62424 |
| | | RPL7A | |
| Heat shock cognate 71 kDa | P11142; | C-1-tetrahydrofolate synthase, | P11586 |
| protein HSPA8 | P54652; | cytoplasmic MTHFD1 | |
| | P48741 | | |
| Triosephosphate isomerase | P60174 | Cysteine-rich secretory protein | Q9H0B8 |
| TPI1 | | LCCL domain-containing 2 | |
| | | CRISPLD2 | |
| Myosin-9 MYH9 | P35579; | Transcription factor BTF3 | P20290 |
| | P35749; | BTF3 | |
| | P35580; | | |
| | A7E2Y1 | | |
| 1-acyl-sn-glycerol-3-phosphate | Q99943 | T-complex protein 1 subunit | P40227 |
| acyltransferase alpha AGPAT1 | | zeta CCT6A | |
| Protein-glutamine gamma- | Q08188 | 40S ribosomal protein S18 | P62269 |
| glutamyltransferase E TGM3 | | RPS18 | |
| Arginase-1 ARG1 | P05089 | 60S ribosomal protein L29 | P47914 |
| | | RPL29 | |
| 78 kDa glucose-regulated | P11021 | Putative lipocalin 1-like | Q5VSP4 |
| protein HSPA5 | | protein 1 LCN1P1 | |
| Carboxypeptidase A1 CPA1 | P15085 | Matrix Gla protein MGP | P08493 |
| Protein S100-A9 S100A9 | P06702 | Elongation factor 1-gamma | P26641 |
| | | EEF1G | |
| Hemoglobin subunit beta HBB | P68871; | Immunoglobulin kappa | A0A0A0 |
| | P02042 | variable 3D-11 IGKV3D-11 | MRZ8 |
| Tubulin beta chain TUBB | P07437; | Histone H2A type 1-B/E | P04908 |
| | Q13509; | HIST1H2AB | |
| | A6NNZ2; | | |
| | Q9H4B7 | | |
| ATP synthase subunit beta, | P06576 | 60S ribosomal protein L30 | P62888 |
| mitochondrial ATP5B | | RPL30 | |
| Keratin, type II cytoskeletal 80 | Q6KB66 | Serine | P34897 |
| | | hydroxymethyltransferase, | |
| | | mitochondrial SHMT2 | |
| Fibrinogen beta chain FGB | P02675 | Proteasome subunit alpha | P60900 |
| | | type-6 PSMA6 | |
| 40S ribosomal protein SA | P08865 | U2 small nuclear | P08579 |
| RPSA | | ribonucleoprotein B″ SNRPB2 | |
| Protein disulfide-isomerase | P07237 | Purine nucleoside | P00491 |
| P4HB | | phosphorylase PNP | |
| Hypoxia-inducible factor 1- | Q16665 | Sex hormone-binding globulin | P04278 |
| alphaHIF1A | | SHBG | |
| Histone H1.5 HIST1H1B | P16401 | High mobility group protein | P09429 |
| | | B1 HMGB1 | |
| ATP synthase subunit alpha, | P25705 | Histidine-rich glycoprotein | P04196 |
| mitochondrial ATP5A1 | | HRG | |
| Histone H3.2 HIST2H3A | Q71DI3; | RNA binding motif protein, | Q96E39; |
| | Q6NXT2 | X-linked-like-1 RBMXL1 | O75526 |
| Apolipoprotein E APOE | P02649 | Ras-related protein Rab-1B | Q9H0U4 |
| | | RAB1B | |
| Keratin, type I cytoskeletal 18 | P05783 | 40S ribosomal protein S3 | P23396 |
| | | RPS3 | |
| Fibrinogen gamma chain FGG | P02679 | Delta(3,5)-Delta(2,4)-dienoyl- | Q13011 |
| | | CoA isomerase, mitochondrial | |
| | | ECH1 | |
| Peroxiredoxin-2 PRDX2 | P32119 | Glia maturation factor gamma | O60234 |
| | | GMFG | |
| Serpin B3 SERPINB3 | P29508; | Rho GDP-dissociation | P52566 |
| | P48594 | inhibitor 2 ARHGDIB | |
| Stress-70 protein, mitochondrial | P38646 | Vitamin K-dependent protein | P07225 |
| HSPA9 | | S PROS1 | |
| 40S ribosomal protein S19 | P39019 | Complement C1s | P09871 |
| RPS19 | | subcomponent C1S | |
| Voltage-dependent anion- | P21796; | Serine/threonine-protein | Q6P5Z2 |
| selective channel protein 1 | Q9Y277 | kinase N3 PKN3 | |
| VDAC1 | | | |
| Immunoglobulin lambda | P0DOY2; | Collagen alpha-1(I) chain | P02452 |
| constant 2 IGLC2 | A0M8Q6 | COL1A1 | |
| Inter-alpha-trypsin inhibitor | QI4624 | Protein SETSIP SETSIP | P0DME0 |
| heavy chain H4 ITIH4 | | | |
| Inter-alpha-trypsin inhibitor | P19827 | Afamin AFM | P43652 |
| heavy chain H1 ITIH1 | | | |
| Trypsin-1 PRSS1 | P07477; | Anoctamin-9 ANO9 | A1A5B4 |
| | Q8NHM4 | | |
| Caspase-14 CASP14 | P31944 | Calpain small subunit 1 | P04632; |
| | | CAPNS1 | Q96L46 |
| Pigment epithelium-derived | P36955 | Lamin-B1 LMNB1 | P20700 |
| factor SERPINF1 | | | |
| Apolipoprotein C-III APOC3 | P02656 | Stathmin STMN1 | P16949 |
| Apolipoprotein C-II APOC2 | P02655 | Rab GDP dissociation | P50395 |
| | | inhibitor beta GDI2 | |
| Hemopexin HPX | P02790 | Parathyroid hormone-related | P12272 |
| | | protein PTHLH | |
| Ubiquitin-40S ribosomal | P62979 | Proteasome subunit alpha | O14818; |
| protein S27a RPS27A | | type-7 PSMA7 | Q8TAA3 |
| Heat shock protein HSP 90-beta | P08238; | Immunoglobulin J chain | P01591 |
| HSP90AB1 | Q58FF7; | JCHAIN | |
| | Q58FF8; | | |
| | Q58FG1; | | |
| | Q12931 | | |
| Galectin-1 LGALS1 | P09382 | Ras-related protein Rab-40C | Q96S21 |
| | | RAB40C | |
| Vitronectin VTN | P04004 | Small nuclear | P62316 |
| | | ribonucleoprotein Sm D2 | |
| | | SNRPD2 | |
| 14-3-3 protein zeta/delta | P63104; | NFX1-type zinc finger- | Q9P2E3 |
| YWHAZ | P31947 | containing protein 1 ZNFX1 | |
| Cofilin-1 CFL1 | P23528; | Calmodulin-1 CALM1 | P0DP23 |
| | Q9Y281 | | |
| Catalase CAT | P04040 | Poly(rC)-binding protein 2 | Q15366; |
| | | PCBP2 | P57721 |
| Proliferating cell nuclear | P12004 | 14-3-3 protein epsilon | P62258 |
| antigen PCNA | | YWHAE | |
| Serpin B12 SERPINB12 | Q96P63 | Protein disulfide-isomerase | P30101 |
| | | A3 PDIA3 | |
| Histone H2B type 1-K | O60814 | Clathrin heavy chain 1 CLTC | Q00610; |
| HIST1H2BK | | | P53675 |
| Alpha-1-antitrypsin SERPINA1 | P01009 | Vitamin D-binding protein GC | P02774 |
| Signal recognition particle 14 | P37108 | Proteasome subunit alpha | P28066 |
| kDa protein SRP14 | | type-5 PSMA5 | |
| Fibrinogen alpha chain FGA | P02671 | 60S ribosomal protein L7 | P18124 |
| | | RPL7 | |
| Collagen alpha-2(VI) chain | P12110 | Adenylyl cyclase-associated | Q01518; |
| COL6A2 | | protein 1 CAP1 | P40123 |
| T-complex protein 1 subunit | P50990 | Keratinocyte proline-rich | Q5T749 |
| theta CCT8 | | protein KPRP | |
| Endoplasmin HSP90B1 | P14625 | X-ray repair cross- | P12956 |
| | | complementing protein 6 | |
| | | XRCC6 | |
| 10 kDa heat shock protein, | P61604 | C-C motif chemokine 5 CCL5 | P13501 |
| mitochondrial HSPE1 | | | |
| Tropomyosin alpha-3 chain | P06753; | Eukaryotic initiation factor | P60842; |
| TPM3 | P09493 | 4A-I EIF4A1 | Q14240; |
| | | | P38919 |
| 60S acidic ribosomal protein P0 | P05388; | Eukaryotic translation | O60841 |
| RPLP0 | Q8NHW5 | initiation factor 5B EIF5B | |
| Nuclease-sensitive element- | P67809; | Keratin, type II cytoskeletal | O95678 |
| binding protein 1 YBX1 | P16989 | 75 | |
| Annexin A6 ANXA6 | P08133 | Heat shock 70 kDa protein 1A | P0DMV8 |
| | | HSPA1A | |
| Transgelin-2 TAGLN2 | P37802 | Haptoglobin-related protein | P00739 |
| | | HPR | |
| Immunoglobulin heavy | P01876; | Tubulin beta-4A chain | P04350 |
| constant alpha 1 IGHA1 | P01877 | TUBB4A | |
| Heterogeneous nuclear | P22626 | HLA class I histocompatibility | P04439; |
| ribonucleoproteins A2/B1 | | antigen, A-3 alpha chain | P01893 |
| HNRNPA2B1 | | HLA-A | |
| L-lactate dehydrogenase B | P07195 | Histone H2B type 1-J | P06899; |
| chain LDHB | | HIST1H2BJ | Q8N257; |
| | | | Q96A08 |
| Antithrombin-III SERPINC1 | P01008 | Keratin, type I cytoskeletal 19 | P08727 |
| Coronin-1A CORO1A | P31146 | Gamma-enolase ENO2 | P09104 |
| Thrombospondin-1 THBS1 | P07996 | Keratin, type II cytoskeletal 3 | P12035 |
| Alpha-fetoprotein AFP | P02771 | Neurofilament heavy | P12036 |
| | | polypeptide NEFH | |
| Protein-glutamine gamma- | P22735 | ADP/ATP translocase 3 | P12236 |
| glutamyltransferase K TGM1 | | SLC25A6 | |
| Corneodesmosin CDSN | Q15517 | Heat shock 70 kDa protein 6 | P17066 |
| | | HSPA6 | |
| Serum amyloid P-component | P02743 | Keratin, type I cytoskeletal 15 | P19012 |
| APCS | | | |
| Fatty acid-binding protein, | Q01469 | Keratin, type II cytoskeletal 4 | P19013 |
| epidermal FABP5 | | | |
| Keratin, type II cytoskeletal 6A | P02538 | Heat shock 70 kDa protein 1- | P34931 |
| | | like HSPA1L | |
| Small nuclear ribonucleoprotein | P62318 | Radixin RDX | P35241 |
| Sm D3 SNRPD3 | | | |
| Immunoglobulin kappa variable | A0A075B6P5 | Keratin, type II cytoskeletal | P48668 |
| 2-28 IGKV2-28 | | 6C | |
| Malate dehydrogenase, | P40926 | Thrombospondin-3 THBS3 | P49746 |
| mitochondrial MDH2 | | | |
| Immunoglobulin kappa variable | P01619; | Tubulin alpha-1B chain | P68363; |
| 3-20 IGKV3-20 | A0A0C4DH25 | TUBA1B | Q9BQE3; |
| | | | Q9NY65; |
| | | | A6NHL2; |
| | | | Q9H853 |
| Keratin, type II cytoskeletal 6B | P04259 | Keratin, type II cytoskeletal | Q3SY84 |
| | | 71 | |
| Thioredoxin-dependent | P30048 | Keratin, type II cytoskeletal | Q5XKE5 |
| peroxide reductase, | | 79 | |
| mitochondrial PRDX3 | | | |
| U6 snRNA-associated Sm-like | Q9Y4Z0 | Putative histone H2B type 2-C | Q6DN03 |
| protein LSm4 LSM4 | | HIST2H2BC | |
| Protein S100-A8 S100A8 | P05109 | Keratin, type II cytoskeletal | Q7RTS7 |
| | | 74 | |
| Nucleophosmin NPM1 | P06748 | Myosin-14 MYH14 | Q7Z406 |
| Alpha-2-antiplasmin | P08697 | WD repeat-containing protein | Q8NI36 |
| SERPINF2 | | 36 WDR36 | |
| Moesin MSN | P26038 | Tubulin beta-6 chain TUBB6 | Q9BUF5 |
| Immunoglobulin heavy | P01860 | Nascent polypeptide- | Q9H009 |
| constant gamma 3 IGHG3 | | associated complex subunit | |
| | | alpha-2 NACA2 | |
| Collagen alpha-1(VI) chain | P12109 | Adipocyte plasma membrane- | Q9HDC9 |
| COL6A1 | | associated protein APMAP | |
| Annexin A1 ANXA1 | P04083 | Keratin, type II cuticular Hb4 | Q9NSB2; |
| | | 84 | O43790 |
| Fibulin-1 FBLN1 | P23142 | Keratin, type II cuticular Hb2 | Q9NSB4 |
| | | 82 | |
| Ras-related C3 botulinum toxin | P15153; | Serpin B13 SERPINB13 | Q9UIV8 |
| substrate 2 RAC2 | P60763 | | |
| Desmocollin-3 DSC3 | Q14574 | Keratin, type II cytoskeletal | Q14CN4 |
| | | 72 | |
| Keratin, type I cytoskeletal 13 | P13646 | Putative heat shock protein | Q58FF6 |
| | | HSP 90-beta 4 HSP90AB4P | |
| Plakophilin-1 PKP1 | Q13835 | Keratin, type II cytoskeletal | Q86Y46 |
| | | 73 | |
| 40S ribosomal protein S8 RPS8 | P62241 | Beta-actin-like protein 2 | Q562R1 |
| | | ACTBL2 | |
| Tubulin beta-4B chain | P68371 | Keratin, type II cytoskeletal 2 | Q01546 |
| TUBB4B | | oral 76 | |
| C4b-binding protein alpha | P04003 | Collagen alpha-3(IV) chain | Q01955 |
| chain C4BPA | | COL4A3 | |
| Protein disulfide-isomerase A6 | Q15084 | Secretory phospholipase A2 | Q13018 |
| PDIA6 | | receptor PLA2R1 | |
| Coactosin-like protein COTL1 | Q14019 | Tubulin beta-2A chain | Q13885 |
| | | TUBB2A | |
| Trypsin-2 PRSS2 | P07478 | T-complex protein 1 subunit | Q92526 |
| | | zeta-2 CCT6B | |
| 60S ribosomal protein L12 | P30050 | HLA class I histocompatibility | P01892; |
| RPL12 | | antigen, A-2 alpha chain | P01891; |
| | | HLA-A | P10321 |
| Cell division control protein 42 | P60953 | Histone H2A.Z H2AFZ | P0C0S5 |
| homolog CDC42 | | | |
| Immunoglobulin gamma-1 | P0DOX5 | Coagulation factor IX F9 | P00740 |
| heavy chain | | | |
| Serine/arginine-rich splicing | Q01130; | Hexokinase-1 HK1 | P19367 |
| factor 2 SRSF2 | Q9BRL6 | | |
| Pregnancy zone protein PZP | P20742 | Bromodomain and WD | Q6RI45 |
| | | repeat-containing protein 3 | |
| | | BRWD3 | |
| Lactotransferrin LTF | P02788 | 40S ribosomal protein S9 | P46781 |
| | | RPS9 | |
| Thyroxine-binding globulin | P05543 | BPI fold-containing family B | Q8TDL5 |
| SERPINA7 | | member 1 BPIFB1 | |
| F-actin-capping protein subunit | P47755 | Peptidyl-prolyl cis-trans | P23284 |
| alpha-2 CAPZA2 | | isomerase B PPIB | |
| 40S ribosomal protein S28 | P62857 | Nucleoprotein TPR TPR | P12270 |
| RPS28 | | | |
| Actin-related protein 2/3 | P59998 | LIM and SH3 domain protein | Q14847 |
| complex subunit 4 ARPC4 | | 1 LASP1 | |
| Immunoglobulin heavy variable | A0A0B4J1X5 | Small ubiquitin-related | Q6EEV6 |
| 3-74 IGHV3-74 | | modifier 4 SUMO4 | |
| 40S ribosomal protein S10 | P46783; | Heterogeneous nuclear | B2RXH8 |
| RPS10 | Q9NQ39; | ribonucleoprotein C-like 2 | |
| | | HNRNPCL2 | |
| T-complex protein 1 subunit | P78371 | Protein Z-dependent protease | Q9UK55 |
| beta CCT2 | | inhibitor SERPINA10 | |
| Annexin A5 ANXA5 | P08758 | Ubiquitin-conjugating enzyme | P61077 |
| | | E2 D3 UBE2D3 | |
| Complement factor H CFH | P08603 | Granulysin GNLY | P22749 |
| Immunoglobulin kappa variable | A0A0C4DH55 | Septin-7 SEPT7 | Q16181 |
| 3D-7 IGKV3D-7 | | | |
| Lumican LUM | P51884 | Heterogeneous nuclear | 014979 |
| | | ribonucleoprotein D-like | |
| | | HNRNPDL | |
| Immunoglobulin lambda-1 light | P0DOX8 | Tetranectin CLEC3B | P05452 |
| chain | | | |
| Calreticulin CALR | P27797 | Proline-rich protein 4 PRR4 | Q16378 |
| Phosphoglycerate mutase 2 | P15259; | Annexin A4 ANXA4 | P09525 |
| PGAM2 | Q8N0Y7 | | |
| Serotransferrin TF | P02787 | Coagulation factor V F5 | P12259 |
| 14-3-3 protein beta/alpha | P31946; | ATP-dependent 6- | Q01813 |
| YWHAB | P61981 | phosphofructokinase, platelet | |
| | | type PFKP | |
| Protein S100-A11 S100A11 | P31949 | Costars family protein | Q9P1F3 |
| | | ABRACL ABRACL | |
| Beta-2-glycoprotein 1 APOH | P02749 | Leukocyte elastase inhibitor | P30740 |
| | | SERPINB1 | |
| Heterogeneous nuclear | P61978 | Neutral alpha-glucosidase AB | Q14697 |
| ribonucleoprotein K HNRNPK | | GANAB | |
| Collagen alpha-3(VI) chain | P12111 | Complement C5 C5 | P01031 |
| COL6A3 | | | |
| Transketolase TKT | P29401 | Actin-related protein 2/3 | O15144 |
| | | complex subunit 2 ARPC2 | |
| Collagen alpha-5(IV) chain | P29400 | Plastin-1 PLS1 | Q14651 |
| COL4A5 | | | |
| Immunoglobulin heavy | P01859; | Putative RNA-binding protein | Q9Y383 |
| constant gamma 2 IGHG2 | P01861 | Luc7-like 2 LUC7L2 | |
| GTP-binding nuclear protein | P62826 | Inosine-5′-monophosphate | P12268 |
| Ran RAN | | dehydrogenase 2 IMPDH2 | |
| Nucleolin NCL | P19338 | Core histone macro-H2A.1 | O75367 |
| | | H2AFY | |
| Plasminogen PLG | P00747; | 60S ribosomal protein L3 | P39023 |
| | Q02325 | RPL3 | |
| Cathepsin D CTSD | P07339 | Vascular cell adhesion protein | P19320 |
| | | 1 VCAM1 | |
| Ras-related protein Rap-1b | P61224; | PRA1 family protein 3 | O75915 |
| RAP1B | A6NIZ1; | ARL6IP5 | |
| | P62834 | | |
| Alpha-actinin-4 ACTN4 | O43707; | Lymphocyte-specific protein 1 | P33241 |
| | P12814; | LSP1 | |
| | P35609; | | |
| | Q9H254 | | |
| Talin-1 TLN1 | Q9Y490; | Polypyrimidine tract-binding | P26599 |
| | Q9Y4G6 | protein 1 PTBP1 | |
| Serpin A12 SERPINA12 | Q8IW75 | Glucose-6-phosphate 1- | P11413 |
| | | dehydrogenase G6PD | |
| Tropomyosin beta chain TPM2 | P07951 | Neuroblast differentiation- | Q09666 |
| | | associated protein AHNAK | |
| | | AHNAK | |
| Prelamin-A/C LMNA | P02545 | Apolipoprotein A-IV APOA4 | P06727 |
| Filaggrin FLG | P20930 | Thymosin beta-4 TMSB4X | P62328 |
| Protein S100-A14 S100A14 | Q9HCY8 | Apolipoprotein M APOM | O95445 |
| Tryptophan--tRNA ligase, | P23381 | Transitional endoplasmic | P55072 |
| cytoplasmic WARS | | reticulum ATPase VCP | |
| DNA replication licensing | P49736 | Coagulation factor X F10 | P00742 |
| factor MCM2 MCM2 | | | |
| 4F2 cell-surface antigen heavy | P08195 | Complement component 1 Q | Q07021 |
| chain SLC3A2 | | subcomponent-binding | |
| | | protein, mitochondrial C1QBP | |
| Carboxypeptidase B CPB1 | P15086 | Rho-related GTP-binding | P08134 |
| | | protein RhoC RHOC | |
| Heat shock protein beta-1 | P04792 | Glutathione S-transferase | P78417 |
| HSPB1 | | omega-1 GSTO1 | |
| Actin-related protein 2/3 | Q9BPX5 | Acidic leucine-rich nuclear | Q92688 |
| complex subunit 5-like protein | | phosphoprotein 32 family | |
| ARPC5L | | member B ANP32B | |
| 60S acidic ribosomal protein P2 | P05387 | 3-hydroxyacyl-CoA | Q99714 |
| RPLP2 | | dehydrogenase type-2 | |
| | | HSD17B10 | |
| T-complex protein 1 subunit | P49368 | Replication protein A 70 kDa | P27694 |
| gamma CCT3 | | DNA-binding subunit RPA1 | |
| Putative small nuclear | A8MWD9 | 40S ribosomal protein S7 | P62081 |
| ribonucleoprotein G-like | | RPS7 | |
| protein 15 SNRPGP15 | | | |
| Multifunctional protein ADE2 | P22234 | Nuclear autoantigenic sperm | P49321 |
| PAICS | | protein NASP | |
| Elongation factor 1-delta | P29692 | Hepatocyte growth factor | Q04756 |
| EEF1D | | activator HGFAC | |
| Bleomycin hydrolase BLMH | Q13867 | 60S ribosomal protein L24 | P83731 |
| | | RPL24 | |
| Nucleoside diphosphate kinase | P22392 | Mitochondrial import receptor | Q9P0U1 |
| B NME2 | | subunit TOM7 homolog | |
| | | TOMM7 | |
| Actin-related protein 3 ACTR3 | P61158 | Ubiquitin-like modifier- | P22314 |
| | | activating enzyme 1 UBA1 | |
| Voltage-dependent anion- | P45880 | Complement component C9 | P02748 |
| selective channel protein 2 | | C9 | |
| VDAC2 | | | |
| Small nuclear ribonucleoprotein | P62304 | Putative 60S ribosomal | Q6NVV1 |
| E SNRPE | | protein L13a protein | |
| | | RPL13AP3 RPL13AP3 | |
| 40S ribosomal protein S14 | P62263 | 60S ribosomal protein L11 | P62913 |
| RPS14 | | RPL11 | |
| Small nuclear | P14678 | Cytochrome c oxidase subunit | P00403 |
| ribonucleoprotein-associated | | 2 MT-CO2 | |
| proteins B and B′ SNRPB | | | |
| Bifunctional purine | P31939 | Tropomodulin-2 TMOD2 | Q9NZR1 |
| biosynthesis protein PURH | | | |
| ATIC | | | |
| Serine/arginine-rich splicing | Q07955 | Fructose-bisphosphate | P09972 |
| factor 1 SRSF1 | | aldolase C ALDOC | |
| Keratin, type II cytoskeletal 8 | P05787; | Tubulin alpha-1A chain | Q71U36; |
| | P14136 | TUBA1A | Q13748; |
| | | | Q6PEY2 |
| Signal transducer and activator | P42224 | ATP synthase subunit f, | P56134 |
| of transcription 1-alpha/beta | | mitochondrial ATP5J2 | |
| STAT1 | | | |
| Retroviral-like aspartic protease | Q53RT3 | Heterogeneous nuclear | Q99729 |
| 1 ASPRV1 | | ribonucleoprotein A/B | |
| | | HNRNPAB | |
| 40S ribosomal protein S23 | P62266 | HLA class II | P01903 |
| RPS23 | | histocompatibility antigen, DR | |
| | | alpha chain HLA-DRA | |
| Heterogeneous nuclear | P07910 | Mucin-5B MUC5B | Q9HC84 |
| ribonucleoproteins C1/C2 | | | |
| HNRNPC | | | |
| Histone H2A type 1 | P0C0S8; | WD repeat-containing protein | O75083 |
| HIST1H2AG | P16104; | 1 WDR1 | |
| | Q8IUE6; | | |
| | Q96QV6 | | |
| Guanine nucleotide-binding | P04899; | Perforin-1 PRF1 | P14222 |
| protein G(i) subunit alpha-2 | A8MTJ3 | | |
| GNAI2 | | | |
| SH3 domain-binding glutamic | Q9H299 | Granzyme M GZMM | P51124 |
| acid-rich-like protein 3 | | | |
| SH3BGRL3 | | | |
| Collagen alpha-2(I) chain | P08123 | Activated RNA polymerase II | P53999 |
| COL1A2 | | transcriptional coactivator p15 | |
| | | SUB1 | |
| Receptor-type tyrosine-protein | P08575 | Ras-related protein Rab-11B | Q15907 |
| phosphatase C PTPRC | | RAB11B | |
| Heterogeneous nuclear | Q00839 | Integrin alpha-L ITGAL | P20701 |
| ribonucleoprotein U HNRNPU | | | |
| Poly(rC)-binding protein 1 | Q15365 | High mobility group protein | P26583 |
| PCBP1 | | B2 HMGB2 | |
| Immunoglobulin lambda | P01700 | HLA class I histocompatibility | P01889; |
| variable 1-47 IGLV1-47 | | antigen, B-7 alpha chain | P30486 |
| | | HLA-B | |
| Copper-transporting ATPase 2 | P35670 | Heterogeneous nuclear | P31943; |
| ATP7B | | ribonucleoprotein H | P55795 |
| | | HNRNPH1 | |
| 40S ribosomal protein S2 RPS2 | P15880 | Peroxiredoxin-6 PRDX6 | P30041 |
| Heterogeneous nuclear | P09651; | 60S ribosomal protein L18 | Q07020 |
| ribonucleoprotein A1 | Q32P51 | RPL18 | |
| HNRNPA1 | | | |
| 14-3-3 protein theta YWHAQ | P27348 | Protein/nucleic acid deglycase | Q99497 |
| | | DJ-1 PARK7 | |
| Eukaryotic translation initiation | Q6IS14 | Adenosylhomocysteinase | P23526 |
| factor 5A-1-like EIF5AL1 | | AHCY | |
| Tubulin alpha-4A chain | P68366 | 40S ribosomal protein S25 | P62851 |
| TUBA4A | | RPS25 | |
| Actin, aortic smooth muscle | P62736 | Tektin-1 TEKT1 | Q969V4 |
| ACTA2 | | | |
| Lactadherin MFGE8 | Q08431 | Putative ubiquitin-conjugating | Q5JXB2 |
| | | enzyme E2 N-like UBE2NL | |
| 60S ribosomal protein L14 | P50914 | Acidic leucine-rich nuclear | P39687 |
| RPL14 | | phosphoprotein 32 family | |
| | | member A ANP32A | |
| Actin-related protein 2/3 | O15511 | Pantetheinase VNN1 | O95497 |
| complex subunit 5 ARPC5 | | | |
| 60S ribosomal protein L22 | P35268 | 60S ribosomal protein L27 | P61353 |
| RPL22 | | RPL27 | |
| Thioredoxin TXN | P10599 | Proteasome activator complex | Q06323 |
| | | subunit 1 PSME1 | |
| Immunoglobulin lambda | A0A075B6K5 | 14-3-3 protein eta YWHAH | Q04917 |
| variable 3-9 IGLV3-9 | | | |
| Myosin regulatory light chain | 014950; | Rho GDP-dissociation | P52565 |
| 12B MYL12B | P24844 | inhibitor 1 ARHGDIA | |
| Myosin light polypeptide 6 | P60660; | Heterogeneous nuclear | P52597 |
| MYL6 | P14649 | ribonucleoprotein F HNRNPF | |
| Galectin-7 LGALS7 | P47929 | T-complex protein 1 subunit | Q99832 |
| | | eta CCT7 | |
| ADP/ATP translocase 2 | P05141 | Heterogeneous nuclear | P51991 |
| SLC25A5 | | ribonucleoprotein A3 | |
| | | HNRNPA3 | |
| Haptoglobin HP | P00738 | Serine/arginine-rich splicing | Q16629 |
| | | factor 7 SRSF7 | |
| Lysozyme C LYZ | P61626 | ATP-dependent RNA helicase | O00148 |
| | | DDX39A DDX39A | |
| Macrophage-capping protein | P40121 | Transformer-2 protein | P62995 |
| CAPG | | homolog beta TRA2B | |
| Glutathione S-transferase P | P09211 | Cartilage oligomeric matrix | P49747 |
| GSTP1 | | protein COMP | |
| Glucose-6-phosphate isomerase | P06744 | Thrombospondin-4 THBS4 | P35443 |
| GPI | | | |
| Chloride intracellular channel | O00299 | Cytokine SCM-1 beta XCL2 | Q9UBD3 |
| protein 1 CLIC1 | | | |
| Heat shock protein HSP 90- | P07900; | Actin-related protein 2/3 | O15143 |
| alpha HSP90AA1 | Q14568; | complex subunit 1B ARPC1B | |
| | Q58FG0 | | |
| Prothrombin F2 | P00734 | Granzyme B GZMB | P10144; |
| | | | P20718 |
| RuvB-like 2 RUVBL2 | Q9Y230 | Interferon gamma IFNG | P01579 |
| Eukaryotic translation initiation | P05198 | Granzyme A GZMA | P12544 |
| factor 2 subunit 1 EIF2S1 | | | |
| Heterogeneous nuclear | O43390 | Filamin-A FLNA | P21333; |
| ribonucleoprotein R HNRNPR | | | O75369 |

In some embodiments, human Granzyme B protein comprises or consists of the amino acid sequence provided in accession number NP_004122 or NP_001332940. In some embodiments, Granzyme B is human Granzyme B (GZMB). In some embodiments, Granzyme B comprises the amino acid sequence MQPILLLLAFLLLPRADAGEIIGGHEAKPHSRPYMAYLMIWDQKSLKRCGGFLIRD DFVLTAAHCWGSSINVTLGAHNIKEQEPTQQFIPVKRPIPHPAYNPKNFSNDIMLLQ LERKAKRTRAVQPLRLPSNKAQVKPGQTCSVAGWGQTAPLGKHSHTLQEVKMTV QEDRKCESDLRHYYDSTIELCVGDPEIKKTSFKGDSGGPLVCNKVAQGIVSYGRNN GMPPRACTKVSSFVHWIKKTMKRY (SEQ ID NO: 8). In some embodiments, human Granzyme B consists of SEQ ID NO: 8. In some embodiments, mouse Granzyme B protein comprises or consists of the amino acid sequence provided in accession number NP_038570. In some embodiments, mouse Granzyme B comprises the amino acid sequence MKILLLLLTLSLASRTKAGEIIGGHEVKPHSRPYMALLSIKDQQPEAICGGFLIREDF VLTAAHCEGSIINVTLGAHNIKEQEKTQQVIPMVKCIPHPDYNPKTFSNDIMLLKLK SKAKRTRAVRPLNLPRRNVNVKPGDVCYVAGWGRMAPMGKYSNTLQEVELTVQ KDRECESYFKNRYNKTNQICAGDPKTKRASFRGDSGGPLVCKKVAAGIVSYGYK DGSPPRAFTKVSSFLSWIKKTMKSS (SEQ ID NO: 16). In some embodiments, mouse Granzyme B consists of SEQ ID NO: 16.

In some embodiments, the lymphocyte lytic granule-secreted protein comprises a signal peptide. In some embodiments, the signal peptide is an endoplasmic reticulum signal peptide (ERSP). In some embodiments, the signal peptide is an endoplasmic reticulum signal sequence (ERSS). In some embodiments, the signal peptide is amino acids 1-18 of SEQ ID NO: 8. In some embodiments, the signal peptide amino acids 1-18 of SEQ ID NO: 16. In some embodiments, the signal peptide comprises amino acids 1-18 of SEQ ID NO: 16. In some embodiments, the signal peptide consists of amino acids 1-18 of SEQ ID NO: 16. In some embodiments, the lymphocyte lytic granule-secreted protein is devoid of a signal peptide. In some embodiments, the granzyme is devoid of a signal peptide. In some embodiments, human Granzyme B is devoid of a signal peptide and comprises or consists of amino acids 19-247 of SEQ ID NO: 8. In some embodiments, a protein devoid of a signal peptide still retains an N-terminal methionine. In some embodiments, human Granzyme B is devoid of a signal peptide and comprises or consists of amino acids 1 and 19-247. In some embodiments, mouse Granzyme B is devoid of the signal peptide and comprises or consists of amino acids 19-247 of SEQ ID NO: 16. In some embodiments, mouse Granzyme B is devoid of the signal peptide and comprises or consists of amino acids 1 and 19-247 of SEQ ID NO: 16. In some embodiments, the granzyme is pro-granzyme. In some embodiments, pro-granzyme comprises an inhibitory dipeptide. In some embodiments, the dipeptide is an N-terminal di-peptide. In some embodiments, the dipeptide is GE. In some embodiments, the dipeptide is amino acids 19-20 of SEQ ID NO: 8. In some embodiments, the dipeptide is amino acids 19-20 of SEQ ID NO: 16. In some embodiments, the Granzyme B is devoid of the inhibitory dipeptide. In some embodiments, human Granzyme B is devoid of the dipeptide and comprises or consists of amino acids 21-247 of SEQ ID NO: 8. In some embodiments, mouse Granzyme B is devoid of the dipeptide and comprises or consists of amino acids 21-247 of SEQ ID NO: 16.

In some embodiments, the chimeric polypeptide comprises a fragment of the lymphocyte lytic granule-secreted protein. In some embodiments, the fragment is a functional fragment. In some embodiments, the function is entry into the ER. In some embodiments, the function is entry into lytic vesicles. In some embodiments, the function is secretion. In some embodiments, the secretion is secretion into an immune synapse. In some embodiments, the function is entry into lytic granules. In some embodiments, the function is inclusion of the chimeric molecule or fragments thereof in lytic granules. In some embodiments, the function is inclusion of the molecule of interest in lytic granules. In some embodiments, the function is inclusion of the protein of interest in lytic granules. In some embodiments, the function is directing the chimeric molecule of the invention into lytic granules. In some embodiments, the function is directing the molecule of interest into lytic granules. In some embodiments, the function is secretion in lytic vesicles. In some embodiments, the function is secretion from lytic vesicles. In some embodiments, the function is not cytotoxicity. In some embodiments, the function is delivery into target cells. In some embodiments, the fragment is a fragment capable of delivering the chimeric polypeptide to target cells. In some embodiments, the fragment is a fragment capable of delivering the chimeric polypeptide to lytic granules. In some embodiments, the fragment is a fragment capable of facilitating inclusion of the chimeric polypeptide and/or the protein of interest in lytic granules. In some embodiments, the fragment is a fragment capable of delivering the chimeric polypeptide to the ER. In some embodiments, the fragment is a non-cytotoxic fragment. In some embodiments, the fragment is a fragment lacking lytic activity. In some embodiments, the fragment lacks the enzymatic domain. In some embodiments, the enzymatic activity is protease activity.

In some embodiments, the fusion protein is naturally trafficked to lytic granules of the modified lymphocytes by the granzyme signaling elements and released upon target recognition to a target cell, through the granzyme pathway. In some embodiments, the fusion protein comprising a lymphocyte lytic granule-targeting moiety/lymphocyte lytic granule-secreted protein/fragment (e.g., granzyme B) and a protein of interest (e.g., CAS nuclease) is naturally trafficked to lytic granules of the modified cells by the granzyme signaling elements, and released upon target recognition to a target cell, through the granzyme pathway. Upon initiation of translation of the fusion protein, comprising, for example, granzyme B and CAS nuclease, the ER signal peptide directs the fusion protein to the ER, where it is co-translationally inserted. As the fusion protein is synthesized in the ER, an N-glycan is added, which targets the translated fusion protein to the Golgi network. In the Golgi, the N-glycan is phosphorylated, and the resultant phosphosugar moiety on the fusion protein then binds to the mannose-6-phosphate receptor, thereby targeting the protein to a lytic granule, where it is sequestered until target cell recognition, and its ensuing release into the immune synapse. The fusion protein/polypeptide may be cleaved in the lytic granule, thus separating the protein of interest from the targeting moiety, such that upon target cell recognition the stand-alone protein of interest is released into the immune synapse. Alternatively, cleavage may occur in the target cell's cytoplasm or another compartment of the target cell. DNA molecules and/or RNA molecules (e.g., sgRNAs) can also be delivered as a complex with the fusion protein or the protein of interest (e.g., an RNP) to the effector cells or the nucleic acid molecules can be directly delivered to the target cells.

In some embodiments, the fragment comprises the signal peptide. In some embodiments, the signal peptide is an endoplasmic reticulum signal peptide. In some embodiments, the fragment is an N-terminal fragment. In some embodiments, the lymphocyte lytic granule-secreted protein comprises at least one glycosylation site. In some embodiments, the lymphocyte lytic granule-secreted protein comprises a plurality of glycosylation sites. In some embodiments, the glycosylation is N-linked glycosylation. In some embodiments, the glycosylation is O-linked glycosylation. In some embodiments, a glycosylation site in human Granzyme B is at amino acid 71 of SEQ ID NO: 8. In some embodiments, a glycosylation site in mouse Granzyme B is at amino acid 71 of SEQ ID NO: 16. In some embodiments, a glycosylation site in human Granzyme B is at amino acid 104 of SEQ ID NO: 8. In some embodiments, a glycosylation site in mouse Granzyme B is at amino acid 182 of SEQ ID NO: 16. In some embodiments, the fragment comprises at least one glycosylation site. In some embodiments, the fragment comprises a plurality of glycosylation sites. In some embodiments, the fragment comprises all glycosylation sites in the lymphocyte lytic granule-secreted protein. In some embodiments, the fragment comprises at least the N-terminal 71 amino acids of human Granzyme B. In some embodiments, the fragment comprises at least the N-terminal 71 amino acids of mouse Granzyme B. In some embodiments, the fragment comprises at least the N-terminal 104 amino acids of human Granzyme B. In some embodiments, the fragment comprises at least the N-terminal 182 amino acids of mouse Granzyme B.

In some embodiments, the fragment comprises at least 10, 20, 25, 30, 40, 50, 60, 70, 75, 80, 90, 100, 110, 120, 125, 130, 140, or 150 amino acids. Each possibility represents a separate embodiment of the invention. In some embodiments, the fragment comprises at least 50 amino acids. In some embodiments, the fragment comprises at least 100 amino acids. In some embodiments, the fragment comprises at least 10, 15, 20, 25, 30, 35, 40, 45, 50, 55, 60, 65, 70, 75, 80, 85, 90, 95, 96, 97, 98, 99 or 100% of the lymphocyte lytic granule-secreted protein. Each possibility represents a separate embodiment of the invention. In some embodiments, the fragment comprises at most 20, 25, 50, 75, 100, 125, 150, 175, 200, 225 or 250 amino acids. Each possibility represents a separate embodiment of the invention. In some embodiments, the fragment comprises at most 25, 30, 35, 40, 45, 50, 55, 60, 65, 70, 75, 80, 85, 90, 95, 96, 97, 98, 99 or 100% of the lymphocyte lytic granule-secreted protein. Each possibility represents a separate embodiment of the invention.

In some embodiments, the lymphocyte lytic granule-secreted protein is a variant of the lymphocyte lytic granule-secreted protein. In some embodiments, a variant is a variant of the naturally-occurring protein. In some embodiments, the variant is a variant of Granzyme B. In some embodiments, a variant is a mutant. In some embodiment, a variant is a mutated form of the naturally-occurring protein. In some embodiments, a mutant is a naturally-occurring protein comprising at least one mutation. In some embodiments, a variant is a cleavable variant. In some embodiments, a variant is an uncleavable variant. In some embodiments, the inhibitory dipeptide of the naturally-occurring protein is uncleavable. In some embodiments, the mutation is mutation of the inhibitory dipeptide to render it uncleavable. In some embodiments, the variant is a non-cytotoxic variant. In some embodiments, the mutation reduces cytotoxicity. In some embodiments, the mutation abolishes cytotoxicity. In some embodiments, the mutant is a non-cytotoxic mutant. In some embodiments, the mutant is a non-lytic mutant. In some embodiments, non-cytotoxic is non-lytic. In some embodiments, non-lytic is non-cytotoxic. In some embodiments, the mutation reduces the enzymatic function of the protein. In some embodiments, the mutation abolishes the enzymatic function of the protein. In some embodiments, the enzymatic function is cleavage. In some embodiments, the cleavage is protein cleavage. In some embodiments, the enzymatic function is a protease function. In some embodiments, the enzymatic function induces lysis. In some embodiments, the enzymatic function induces cell death. In some embodiments, the variant is an inactive variant. In some embodiments, the variant is an inert variant. In some embodiments, the variant is a non-cytotoxic variant. In some embodiments, non-cytotoxic variant is not able to induce apoptosis in a target cell. In some embodiments, the variant is a non-lytic variant. In some embodiments, the variant is not able to induce apoptosis in a target cell. In some embodiments, the variant is a homolog. In some embodiments, the homolog is not cytotoxic in humans. In some embodiments, the homolog has reduced cytotoxicity in humans.

In some embodiments, the lymphocyte lytic granule-secreted protein is not cytotoxic. In some embodiments, the lymphocyte lytic granule-secreted protein is not lytic. In some embodiments, the lymphocyte lytic granule-secreted protein is not enzymatically active. In some embodiments, the lymphocyte lytic granule-secreted protein is inert. In some embodiments, the lymphocyte lytic granule-secreted protein is inactivated. In some embodiments, inactivity is loss of enzymatic activity. In some embodiments, enzymatic activity is protease activity. In some embodiments, inclusion of the inhibitory dipeptide renders the granzyme inactive. In some embodiments, the inhibitory dipeptide has been mutated. In some embodiments, the mutation renders the inhibitory dipeptide uncleavable resulting in an activatable form of the granzyme. In some embodiments, the inhibitory dipeptide is a mutant uncleavable inhibitory dipeptide. In some embodiments, the mutation is a mutation of amino acid 19 of SEQ ID NO: 8. In some embodiments, the mutation is a mutation of amino acid 19 of SEQ ID NO: 16. In some embodiments, the mutation is a mutation of the first amino acid of the dipeptide. In some embodiments, the mutation is a mutation of amino acid 20 of SEQ ID NO: 8. In some embodiments, the mutation is a mutation of amino acid 20 of SEQ ID NO: 16. In some embodiments, the mutation is a mutation of the second amino acid of the dipeptide. In some embodiments, mutation is a mutation to alanine. In some embodiments, both amino acids of the dipeptide are mutated. In some embodiments, amino acids 19 and 20 of SEQ ID NO: 8 are mutated. In some embodiments, amino acids 19 and 20 of SEQ ID NO: 16 are mutated. In some embodiments, the dipeptide is mutated to AA.

In some embodiments, the lymphocyte lytic granule-secreted protein and the protein of interest are separated by a linker. In some embodiments, the lymphocyte lytic granule-secreted protein and the protein of interest are linked by a linker. In some embodiments, the lymphocyte lytic granule-secreted protein and the protein of interest are joined by a linker. In some embodiments, the linker is a peptide bond. In some embodiments, the linker is an amino acid linker. In some embodiments, the linker is a chemical linker. In some embodiments, the linker is a flexible linker. In some embodiments, the linker comprises a plurality of amino acids. In some embodiments, the linker comprises at least, 1, 2, 3, 4, 5, 6, 7, 8, 9 or 10 amino acids. Each possibility represents a separate embodiment of the invention. In some embodiments, the linker comprises at least 5 amino acids. In some embodiments, the linker comprises at most 5, 10, 15, 20, 25, 30, 35, 40, 45 or 50 amino acids. Each possibility represents a separate embodiment of the invention. In some embodiments, the linker comprises at most 25 amino acids.

In some embodiments, the linker is a glycine-serine linker. In some embodiments, the linker comprises or consists of (GS)n, wherein n is an integer from 1-10. In some embodiments, the linker is GSGSGSGSGS (SEQ ID NO: 9). In some embodiments, the linker comprises or consists of GGGGS (SEQ ID NO: 10). In some embodiments, the linker comprises or consists of (GGGGS)n, wherein n is an integer from 1-5. In some embodiments, the linker is GGGGSGGGGSGGGGSGGGGS (SEQ ID NO: 11). In some embodiments, the linker comprises or consists of GGS (SEQ ID NO: 43). In some embodiments, the linker comprises or consists (GGS)n, wherein n is an integer from 1-10. In some embodiments, the linker is GGSGGSGGSGGS (SEQ ID NO: 44). In some embodiments, the linker is SGFANELGPRLMGK (SEQ ID NO: 72). In some embodiments, the OLLAS linker consists of SEQ ID NO: 72.

In some embodiments, the linker is a cleavable linker. It will be understood by a skilled artisan that it may be advantageous for the protein of interest to be separated from the lymphocyte lytic granule-secreted protein to better enable the protein to function. However, such separation should only occur after the lymphocytic lytic granule-secreted protein has served its function of facilitating delivery of the protein of interest to the lytic granule and/or the target cell. It will be understood by a skilled artisan that if cleavage occurs in the lytic granule the protein of interest would still be transferred to the target cell. Upon engagement of a lymphocyte to a target cell, all the proteins present in the granule (including the protein of interest even if separate from the lymphocytic lytic granule-secreted protein) passively diffuse to the target cell by the process of lytic granule secretion. This type of protein transfer at the immune synapse is well known in the art. In some embodiments, the linker is a pH-dependent cleavable linker. In some embodiments, the linker is cleavable at acidic pH. It will be understood by a skilled artisan that the lytic granule comprises an acidic pH. A linker that cleaves at acidic pH will ensure that the lymphocyte lytic granule-secreted protein or fragment thereof remains connected to the protein of interest, at least until they reach the lytic vesicle. Once the lytic granule reaches the target cell, the protein of interest has been released from the lymphocyte lytic-granule-secreted protein and is able to function freely in the target cell. In some embodiments, an acidic pH cleavable linker comprises an aspartic acid-proline (DP) dipeptide. In some embodiments, the linker comprises the amino acid sequence RARDPPVAT (SEQ ID NO: 12). In some embodiments, the linker consists of SEQ ID NO: 12. In some embodiments, the linker comprises the amino acid sequence DXDPHF (SEQ ID NO: 13). In some embodiments, the linker consists of SEQ ID NO: 13. In some embodiments, the linker comprises the amino acid sequence GTGDP (SEQ ID NO: 14). In some embodiments, the linker consists of SEQ ID NO: 14.

In some embodiments, the linker is a Cathepsin cleavable linker. Non-limiting examples of Cathepsin include, but are not limited to Cathepsin L, Cathepsin B and Cathepsin C. Cathepsins are known to be active in the lytic granules and thus are also useful for targeted cleavage of the linker. In some embodiments, the linker comprises the dipeptide VA. In some embodiments, the linker consists of the dipeptide VA. In some embodiments, the linker comprises the dipeptide GE. In some embodiments, the linker consists of the dipeptide GE.

In some embodiments, the linker is cleavable in the cytoplasm. In some embodiments, a cytoplasmic cleavable linker is cleavable at cytoplasmic glutathione levels. It will be understood by a skilled artisan that inclusion of an ER signal peptide will trigger co-translational entry of the chimeric polypeptide into the ER, thereby sequestering the linker from the cleavage-inducing environment of the lymphocyte's cytoplasm. Cytoplasmic cleavage would thus be prevented until the fusion protein's arrival in the target cell's cytoplasm, wherein the protein of interest is set free to exert its function. In some embodiments, the cleavable linker comprises the first 11 amino acids of mCherry. In some embodiments, the cleavable linker comprises amino acids 2-11 of mCherry. In some embodiments, mCherry comprises or consists of MVSKGEEDNMAIIKEFMRFKVHMEGSVNGHEFEIEGEGEGRPYEGTQTAKLKVTK GGPLPFAWDILSPQFMYGSKAYVKHPADIPDYLKLSFPEGFKWERVMNFEDGGVV TVTQDSSLQDGEFIYKVKLRGTNFPSDGPVMQKKTMGWEASSERMYPEDGALKG EIKQRLKLKDGGHYDAEVKTTYKAKKPVQLPGAYNVNIKLDITSHNEDYTIVEQY ERAEGRHSTGGMDELYK (SEQ ID NO: 1). In some embodiments, mCherry comprises or consists of SEQ ID NO: 1. In some embodiments, the linker comprises VSKGEEDNMA (SEQ ID NO: 2). In some embodiments, the linker consists of SEQ ID NO: 2. In some embodiments, SEQ ID NO: 2 is a cleavable linker. In some embodiments, mCherry is crmCherry. In some embodiments, crmCherry lacks the first 11 amino acids of mCherry. In some embodiments, crmCherry comprises or consists of SEQ ID NO: 3. In some embodiments, crmCherry is encoded by SEQ ID NO: 7.

In some embodiments, the molecule of interest is a therapeutic molecule. In some embodiments, the molecule is a drug. In some embodiments, the molecule is a biologic. In some embodiments, the molecule is a biologic molecule. In some embodiments, the molecule is a nucleic acid molecule. In some embodiments, the molecule comprises a nucleic acid molecule. In some embodiments, the nucleic acid is DNA. In some embodiments, the nucleic acid is RNA. In some embodiments, the nucleic acid is DNA or RNA. In some embodiments, the molecule is a protein-RNA complex. In some embodiments, the molecule is an RNP. In some embodiments, the molecule is a protein. In some embodiments, the molecule is a protein fragment. In some embodiments, the molecule is a non-natural molecule. In some embodiments, the molecule is a naturally-occurring molecule. In some embodiments, the molecule is a modified form of a naturally-occurring molecule. In some embodiments, the molecule is an enzyme or enzymatically active. In some embodiments, the molecule is a binding molecule. In some embodiments, the molecule of interest is the cargo. In some embodiments, the molecule of interest is a therapeutic agent. In some embodiments, the therapeutic agent is a therapeutic proteinaceous agent.

In some embodiments, the molecule of interest is a protein of interest. In some embodiments, the protein of interest is a therapeutic agent. In some embodiments, the protein of interest is a fusion protein. In some embodiments, the protein of interest is a cytoplasmic protein. In some embodiments, the protein of interest is active in the cytoplasm. In some embodiments, the protein of interest is a nuclear protein. In some embodiments, the protein of interest is active in the nucleus.

In some embodiments, the protein of interest is not a membrane protein. In some embodiments, the protein of interest is not a lymphocyte membrane protein. In some embodiments, the protein of interest is not naturally a membrane protein. In some embodiments, the protein of interest is not naturally membranal in lymphocytes. In some embodiments, the cell does not comprise the molecule of interest within its cellular membrane. In some embodiments, the cell does not comprise the molecule of interest conjugated to its cellular membrane. In some embodiments, the cell does not comprise the therapeutic agent within its cellular membrane. In some embodiments, the cellular membrane is the plasma membrane. In some embodiments, the protein of interest does not comprise a transmembrane domain.

In some embodiments, the protein of interest is not a naturally secreted protein. In some embodiments, the protein of interest does not comprise a signal peptide. In some embodiments, the protein of interest does not naturally comprise a signal peptide. In some embodiments, the protein of interest is not a receptor ligand. In some embodiments, the protein of interest does not bind a receptor. In some embodiments, the receptor is a surface receptor. In some embodiments, the receptor is a plasma membrane receptor. In some embodiments, the protein of interest is not a targeting protein. In some embodiments, the protein of interest induces an effect in the target cell. In some embodiments, the protein of interest induces a therapeutic effect in the target cell. In some embodiments, the protein of interest binds a surface protein and activates or inhibits that surface protein. In some embodiments, the protein of interest is an antagonist. In some embodiments, the protein of interest is an agonist. In some embodiments, the protein of interest binds a surface protein and induces signaling through that bound receptor. In some embodiments, the protein of interest is a surface receptor ligand. In some embodiments, the protein of interest is a protein that naturally binds its target. In some embodiments, the protein of interest is an antibody or antigen-binding fragment thereof. In some embodiments, the protein of interest is a synthetic binding agent. In some embodiments, the protein of interest is a single chain antibody. In some embodiments, the protein of interest is a single domain antibody. In some embodiments, the protein of interest is a VHH.

In some embodiments, the protein of interest is not a viral protein. In some embodiments, the protein of interest is not a viral envelope protein. In some embodiments, the protein of interest is not a viral penetration protein. In some embodiments, the protein of interest is not a viral spike protein. In some embodiments, the therapeutic agent is not a full virus. In some embodiments, the therapeutic agent is not a vaccine. In some embodiments, the therapeutic agent is not an oncolytic virus. In some embodiments, the therapeutic agent is not a viral particle. In some embodiments, the therapeutic agent is not a viral genome. In some embodiments, the therapeutic agent is a viral genome-editing protein. In some embodiments, the protein of interest is an antigen-binding protein or fragment thereof. In some embodiments, the antigen is not a surface antigen. In some embodiments, the antigen is a cytoplasmic antigen. In some embodiments, the antigen is a nuclear antigen. In some embodiments, the antigen is an internal antigen. In some embodiments, the antigen is internal to a target cell. In some embodiments, the molecule of interest is not nanoparticle conjugated. In some embodiments, the molecule of interest is not encapsulated. In some embodiments, encapsulated is nanoparticle encapsulated. In some embodiments, the chimeric molecule is not nanoparticle conjugated. In some embodiments, the chimeric molecule is not encapsulated.

In some embodiments, the protein of interest is a protein fragment. In some embodiments, the fragment comprises at least one functional domain. In some embodiments, the fragment is a therapeutic fragment. In some embodiments, the fragment comprises at least 10, 15, 20, 25, 30, 35, 40, 45, 50, 60, 70, 75, 80, 90, or 100 amino acids. Each possibility represents a separate embodiment of the invention. In some embodiments, the fragment comprises at least 25 amino acids. In some embodiments, the fragment is not the complete protein. In some embodiments, the fragment lacks a cleavage site. In some embodiments, the fragment lacks a cleavage site that could result in the protein of interest being cleaved from the lymphocytic lytic granule-secreted protein prematurely. In some embodiments, prematurely is before entry to the ER. In some embodiments, prematurely is before entry into the lytic granule. In some embodiments, the protein of interest is not a lymphocytic lytic granule-secreted protein. In some embodiments, the protein of interest is not a targeting moiety.

In some embodiments, the molecule of interest comprises a molecular weight greater than 25, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 180, 190 or 200 kilodaltons (kDa). Each possibility represents a separate embodiment of the invention. In some embodiments, the molecule of interest comprises a molecular weight greater than 25 kDa. In some embodiments, the molecule of interest comprises a molecular weight greater than 28 kDa. In some embodiments, the molecule of interest comprises a molecular weight greater than 50 kDa. In some embodiments, the molecule of interest comprises a molecular weight greater than 75 kDa. In some embodiments, the molecule of interest comprises a molecular weight greater than 100 kDa. In some embodiments, the molecule of interest comprises a molecular weight greater than 125 kDa. In some embodiments, the molecule of interest comprises a molecular weight greater than 150 kDa. In some embodiments, the molecule of interest comprises a molecular weight greater than 160 kDa. In some embodiments, the molecule of interest comprises a molecular weight greater than 190 kDa. In some embodiments, the molecule of interest comprises a molecular weight between 25-300, 25-250, 25-200, 25-190, 25-175, 25-165, 28-300, 28-250, 28-200, 28-190, 28-175, 28-165, 30-300, 30-300, 30-200, 30-190, 30-175, 30-165, 50-300, 50-500, 50-200, 50-190, 50-175, 50-165, 75-300, 75-750, 75-200, 75-190, 75-175, 75-165, 100-300, 100-1000, 100-200, 100-190, 100-175, or 100-165 kDa. Each possibility represents a separate embodiment of the invention. In some embodiments, the molecule of interest comprises a molecular weight between 25-300 kDa. In some embodiments, the molecule of interest comprises a molecular weight between 25-200 kDa. In some embodiments, the molecule of interest comprises a molecular weight between 25-190 kDa. In some embodiments, the molecule of interest comprises a molecular weight between 25-165 kDa. In some embodiments, the molecule of interest comprises a molecular weight between 30-300 kDa. In some embodiments, the molecule of interest comprises a molecular weight between 30-200 kDa. In some embodiments, the molecule of interest comprises a molecular weight between 30-190 kDa. In some embodiments, the molecule of interest comprises a molecular weight between 30-165 kDa. In some embodiments, the molecule of interest comprises a molecular weight between 50-300 kDa. In some embodiments, the molecule of interest comprises a molecular weight between 50-200 kDa. In some embodiments, the molecule of interest comprises a molecular weight between 50-190 kDa. In some embodiments, the molecule of interest comprises a molecular weight between 50-165 kDa. In some embodiments, the molecule of interest comprises a molecular weight between 100-300 kDa. In some embodiments, the molecule of interest comprises a molecular weight between 100-200 kDa. In some embodiments, the molecule of interest comprises a molecular weight between 100-190 kDa. In some embodiments, the molecule of interest comprises a molecular weight between 100-165 kDa.

In some embodiments, the molecule of interest is not cytotoxic. In some embodiments, the chimeric molecule is not cytotoxic. In some embodiments, the lymphocytic lytic granule-secreted protein is not cytotoxic. In some embodiments, the lymphocytic lytic granule-secreted protein is not cytotoxic, and the molecule of interest is cytotoxic. In some embodiments, the lymphocytic lytic granule-secreted protein is cytotoxic, and the molecule of interest is cytotoxic. In some embodiments, the lymphocytic lytic granule-secreted protein is cytotoxic, and the molecule of interest is not cytotoxic. In some embodiments, the lymphocytic lytic granule-secreted protein is not cytotoxic, and the molecule of interest is not cytotoxic. In some embodiments, a cytotoxic protein is a protein that can induce apoptosis in a cell. In some embodiments, the protein of interest is cytotoxic. In some embodiments, the protein of interest is cytotoxic when delivered to the interior of a target cell. In some embodiments, the protein of interest is not cytotoxic when delivered to the surface of a target cell. In some embodiments, the interior is the cytoplasm. In some embodiments, the interior is the nucleus. In some embodiments, the interior is the mitochondria. In some embodiments, cytotoxic is lytic.

In some embodiments, the molecule of interest is an RNA-protein complex. In some embodiments, an RNA-protein complex is a ribonucleoprotein (RNP). The term “ribonucleotide” or “ribonucleic acid” (RNA), refers to a modified or unmodified nucleotide or polynucleotide comprising at least one ribonucleotide unit. A ribonucleotide unit comprises a hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

In some embodiments, the protein is a nuclease. In some embodiments, the molecule of interest is a genome-editing complex. In some embodiments, the protein of interest is a genome-editing protein. In some embodiments, editing is modifying. In some embodiments, a genome-editing protein is selected from the group consisting of a clustered Regularly Interspaced Short Palindromic Repeats (CRISPR)-associated nuclease, a Zinc-finger nuclease (ZFNs), a meganuclease and a transcription activator-like effector nuclease (TALEN). In some embodiments, the genome-editing protein is a meganuclease. In some embodiments, the genome-editing protein is a natural meganuclease. In some embodiments, the genome-editing protein is a modified/engineered meganuclease. In some embodiments, the meganuclease is specific to a DNA target sequence of a mammalian genome. In some embodiments, the meganuclease is specific to a DNA target sequence of a mammalian gene. In some embodiments, the meganuclease is a PCSK9-specific meganuclease. In some embodiments, the PCSK9-specific meganuclease comprises the amino acid sequence MHMNTKYNKEFLLYLAGFVDGDGSIFARIKPSQRSKFKHKLHLVFAVYQKTQRR WFLDKLVDEIGVGYVLDSGSVSFYSLSEIKPLHNFLTQLQPFLKLKQKQANLVLKII EQLPSAKESPDKFLEVCTWVDQIAALNDSKTRKTTSETVRAVLDSLPGSVGGLSPS QASSAASSASSSPGSGISEALRAGAGSGTGYNKEFLLYLAGFVDGDGSIYARIKPVQ RAKFKHELVLGFDVTQKTQRRWFLDKLVDEIGVGYVYDKGSVSAYRLSQIKPLH NFLTQLQPFLKLKQKQANLVLKIIEQLPSAKESPDKFLEVCTWVDQIAALNDSKTR KTTSETVRAVLDSLSEKKKSSP (SEQ ID NO: 53). In some embodiments, the PCSK9-specific meganuclease is encoded by the nucleotide sequence provided in SEQ ID NO: 54.

In some embodiments, the genome-editing protein is a CRISPR-associated protein. In some embodiments, the CRISPR-associated protein is CRISPR-associated protein 9 (Cas9). In some embodiments, the CRISPR-associated protein is Cas9 or a Cas9 ortholog. In some embodiments, the CRISPR-associated protein is Cas9 or a Cas9 variant. In some embodiments, the CRISPR-associated protein is Cas9 or a Cas9 homolog.

In some embodiments, the CRISPR-associated protein is a CRISPR-associated nuclease. In some embodiments, the CRISPR-associated nuclease is a CPF1 nuclease. In some embodiments, the CRISPR-associated nuclease is a Cas12a nuclease. In some embodiments, the CRISPR-associated nuclease is a Cas13a nuclease. In some embodiments, the CRISPR-associated nuclease is a CasI nuclease. In some embodiments, the CRISPR-associated nuclease is a CasIB nuclease. In some embodiments, the CRISPR-associated nuclease is a Cas2 nuclease. In some embodiments, the CRISPR-associated nuclease is a Cas3 nuclease. In some embodiments, the CRISPR-associated nuclease is a Cas4 nuclease. In some embodiments, the CRISPR-associated nuclease is a Cas5 nuclease. In some embodiments, the CRISPR-associated nuclease is a Cas6 nuclease. In some embodiments, the CRISPR-associated nuclease is a Cas7 nuclease. In some embodiments, the CRISPR-associated nuclease is a Cas8 nuclease. In some embodiments, the CRISPR-associated nuclease is a CaslOO nuclease. In some embodiments, the CRISPR-associated nuclease is a Csyl nuclease. In some embodiments, the CRISPR-associated nuclease is a Csy2 nuclease. In some embodiments, the CRISPR-associated nuclease is a Csy3 nuclease. In some embodiments, the CRISPR-associated nuclease is a Cse1 nuclease. In some embodiments, the CRISPR-associated nuclease is a Cse2 nuclease. In some embodiments, the CRISPR-associated nuclease is a Csc1 nuclease. In some embodiments, the CRISPR-associated nuclease is a Csc2 nuclease. In some embodiments, the CRISPR-associated nuclease is a Csa5 nuclease. In some embodiments, the CRISPR-associated nuclease is a Csn2 nuclease. In some embodiments, the CRISPR-associated nuclease is a Csm2 nuclease. In some embodiments, the CRISPR-associated nuclease is a Csm3 nuclease. In some embodiments, the CRISPR-associated nuclease is a Csm4 nuclease. In some embodiments, the CRISPR-associated nuclease is a Csm5 nuclease. In some embodiments, the CRISPR-associated nuclease is a Csm6 nuclease. In some embodiments, the CRISPR-associated nuclease is a Cmrl nuclease. In some embodiments, the CRISPR-associated nuclease is a Cmr3 nuclease. In some embodiments, the CRISPR-associated nuclease is a Cmr4 nuclease. In some embodiments, the CRISPR-associated nuclease is a Cmr5 nuclease. In some embodiments, the CRISPR-associated nuclease is a Cmr6 nuclease. In some embodiments, the CRISPR-associated nuclease is a Csbl nuclease. In some embodiments, the CRISPR-associated nuclease is a Csb2 nuclease. In some embodiments, the CRISPR-associated nuclease is a Csb3 nuclease. In some embodiments, the CRISPR-associated nuclease is a Csxl7 nuclease. In some embodiments, the CRISPR-associated nuclease is a Csxl4 nuclease. In some embodiments, the CRISPR-associated nuclease is a CsxlO nuclease. In some embodiments, the CRISPR-associated nuclease is a Csxl6 nuclease. In some embodiments, the CRISPR-associated nuclease is a CsaX nuclease. In some embodiments, the CRISPR-associated nuclease is a Csx3 nuclease. In some embodiments, the CRISPR-associated nuclease is a Csxl nuclease. In some embodiments, the CRISPR-associated nuclease is a Csxl5 nuclease. In some embodiments, the CRISPR-associated nuclease is a Csfl nuclease. In some embodiments, the CRISPR-associated nuclease is a Csf2 nuclease. In some embodiments, the CRISPR-associated nuclease is a Csf3 nuclease. In some embodiments, the CRISPR-associated nuclease is a Csf4 nuclease. In some embodiments, the CRISPR-associated nuclease is a Prime Editor 1 (PE1) nickase. In some embodiments, PE1 is encoded by SEQ ID NO: 27. In some embodiments, the CRISPR-associated nuclease is a Prime Editor 2 (PE2) nickase. In some embodiments, PE2 is encoded by SEQ ID NO: 28. In some embodiments, the CRISPR-associated nuclease is a Prime Editor 3 (PE3) nickase. In some embodiments, the CRISPR-associated nuclease is a MAD7 nuclease. In some embodiments, the CRISPR-associated nuclease is a CRISPRi nuclease (CRISPR interference). In some embodiments, the CRISPR-associated nuclease is a CRISPRa nuclease (CRISPR activation). In some embodiments, the CRISPR-associated nuclease is a class 1 CRISPR nuclease. In some embodiments, the genome editing protein is a homing endonuclease. In some embodiments, the genome editing protein is a meganucleases. In some embodiments, meganucleases are available for producing targeted genome perturbations. In some embodiments, one or more of the above endonucleases or homologs thereof, a recombination of the naturally-occurring molecules thereof, codon-optimized version thereof, or modified versions thereof and combination thereof are employed.

In some embodiments, the Cas9 is Streptococcus pyogenes Cas9 (SpCas9). In some embodiments, the Cas9 is Campylobacter jejuni Cas9. In some embodiments, Campylobacter jejuni Cas9 comprises SEQ ID NO: 19. In some embodiments, Campylobacter jejuni Cas9 consists of SEQ ID NO: 19. In some embodiments, the Cas9 is Streptococcus pyogenes serotype M1 Cas9. In some embodiments, SpCas9 is Sp serotype M1 Cas9. In some embodiments, Streptococcus pyogenes serotype M1 Cas9 comprises SEQ ID NO: 20. In some embodiments, Streptococcus pyogenes serotype M1 Cas9 consists of SEQ ID NO: 20. In some embodiments, the Cas9 is Staphylococcus aureus Cas9. In some embodiments, Staphylococcus aureus Cas9 comprises SEQ ID NO: 21. In some embodiments, Staphylococcus aureus Cas9 consists of SEQ ID NO: 21. In some embodiments, the Cas9 is Neisseria meningitidis serogroup C (strain 8013) Cas9. In some embodiments, Neisseria meningitidis serogroup C (strain 8013) Cas9 comprises SEQ ID NO: 22. In some embodiments, Neisseria meningitidis serogroup C (strain 8013) Cas9 consists of SEQ ID NO: 22. In some embodiments, the Cas9 is Geobacillus stearothermophilus Cas9. In some embodiments, Geobacillus stearothermophilus Cas9 comprises SEQ ID NO: 23. In some embodiments, Geobacillus stearothermophilus Cas9 consists of SEQ ID NO: 23. In some embodiments, the CRISPR associated protein is Cas12a. In some embodiments, the Cas12a is CRISPR-associated endonuclease Cas12a Francisella tularensis subsp. novicida (strain U112). In some embodiments, Cas12a comprises SEQ ID NO: 24. In some embodiments, Cas12a consists of SEQ ID NO: 24. In some embodiments, Cas12a is CRISPR-associated endonuclease Cas12a OS=Acidaminococcus sp. (strain BV3L6). In some embodiments, Cas12a comprises SEQ ID NO: 25. In some embodiments, Cas12a consists of SEQ ID NO: 25. In some embodiments, Cas12a is Cas12a/Cpf1. In some embodiments, Cas12a is type V CRISPR-associated protein Cas12a/Cpf1 of Lachnospiraceae bacterium ND2006. In some embodiments, Cas12a comprises SEQ ID NO: 26. In some embodiments, Cas12a consists of SEQ ID NO: 26. In some embodiments, the genome editing protein is prime editor 1 (PE1). In some embodiments, PE1 consists of SEQ ID NO: 73. In some embodiments, the genome editing protein is prime editor 2 (PE2). In some embodiments, PE2 consists of SEQ ID NO: 74. In some embodiments, the protein of interest is selected from SEQ ID Nos: 19-26 and 73, 74.

In some embodiments, the genome editing protein is one of those provided herein above. In some embodiments, the genome editing protein comprises one of the sequences provided hereinabove. In some embodiments, the genome editing protein consists of one of the sequences provided hereinabove. In some embodiments, the genome editing protein corresponds to one of the sequences provided hereinabove. In some embodiments, the genome editing protein is a variant or one of the protein/sequences provided hereinabove. It will be understood by a skilled artisan that the natural protein can be modified, or a variant can be generated, that is optimized for expression in a mammal or specifically a human. Such optimization can include codon optimization, structural optimization, optimization of expression, optimization of genome editing, optimization of specificity, and other optimizations. Any such optimization known in the art may be used, and any variant sequence corresponding or derived from those provided hereinabove may be employed.

In some embodiments, the RNA in the RNA-protein complex is a guide RNA (gRNA). In some embodiments, the RNA is a gRNA. In some embodiments, the guide RNA is a single guide RNA (sgRNA). In some embodiments the RNA is chemically modified. In some embodiments, the modification increases stability of the RNA. In some embodiments, the modification increases half-life of the RNA. In some embodiments, the modification decreases degradation of the RNA. In some embodiments, the modification decreases cleavage of the RNA. In some embodiments, the modification decreases immunogenicity of the RNA. In some embodiments, the modification decreases off-target effects. Chemical modifications of RNA are well known in the art and include, for example, 2-O-methyl analogs, 2-fluoro analogs, 2-MOE and phosphorothioate, and any such modification may be employed. In some embodiments, the RNA targets a target protein of interest. In some embodiments, the target protein of interest is a disease-associated protein. In some embodiments, the target protein of interest is a disease-causing protein. In some embodiments, modification of the target protein of interest has a therapeutic benefit. In some embodiments, a disease is characterized by a mutation in the target protein of interest. In some embodiments, a disease is caused by a mutation in the target protein of interest. In some embodiments, a disease is treatable by gene editing of the target protein of interest. In some embodiments, a disease is treatable by altering regulation of the target protein of interest. In some embodiments, regulation is down-regulation. In some embodiments, regulation is up-regulation.

In some embodiment, the protein of interest further comprises a localization sequence. It will be understood by a skilled artisan that proteins that are intended to act in a particular subcellular location may have enhanced function if they are targeted to that location. In some embodiments, the localization sequence is a nuclear localization sequence (NLS). In some embodiments, the localization sequence is a mitochondrial localization sequence (MILS). In some embodiments, a localization sequence is capable of transporting a protein to which it is linked from the cytoplasm to a target subcellular location. In some embodiments, the location is an organelle. In some embodiments, the organelle comprises a membrane and the sequence is capable of transport across or through the membrane. In some embodiments, the localization sequence is attached to the N-terminus of the protein of interest. In some embodiments, the localization sequence is attached to the C-terminus of the protein of interest. In some embodiments, the localization sequence is internal to the protein of interest. In some embodiments, the localization sequence is directly conjugated to the protein of interest. In some embodiments, the localization sequence is conjugated to the protein of interest by a linker. In some embodiments, the protein of interest is operably linked to the localization sequence. In some embodiments, the localization sequence is operably linked to the protein of interest.

Nucleic Acid Molecules

By another aspect, there is provided a polynucleotide encoding a chimeric molecule of the invention.

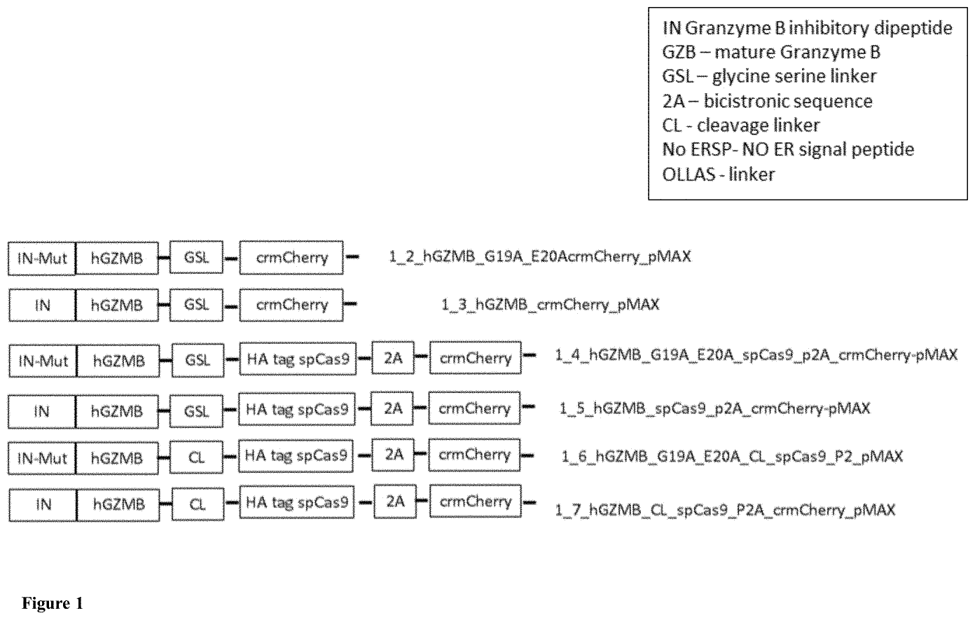

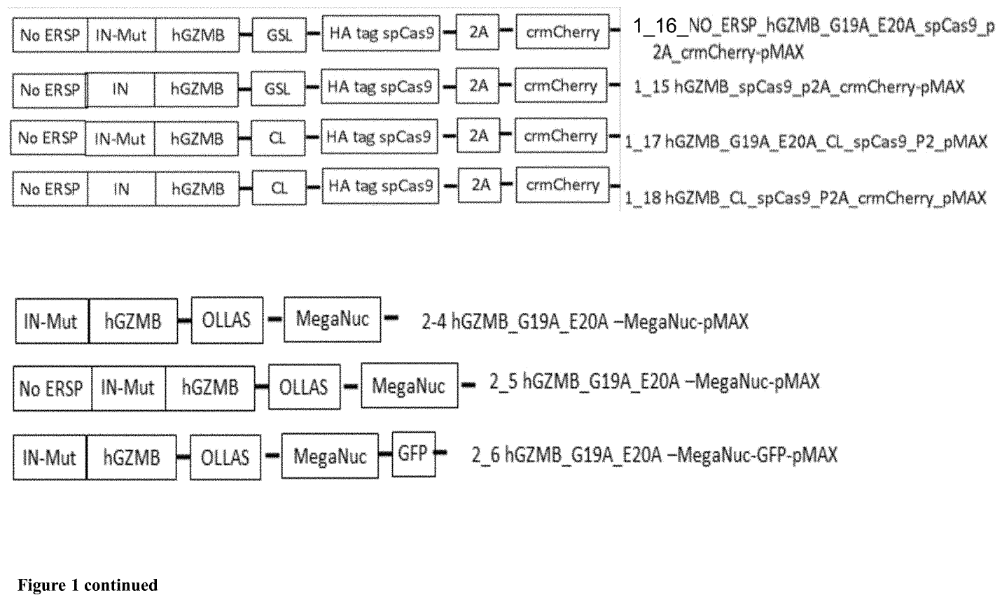

Schematics of exemplary nucleic acid molecules are provided in FIG. 1.

In some embodiments, the polynucleotide is a polynucleotide molecule. In some embodiments, the polynucleotide is a vector. In some embodiments, the polynucleotide is an expression vector. In some embodiments, the expression vector is configured for expression in a mammalian cell. In some embodiments, configured for expression is capable of expression. In some embodiments, the expression vector is configured for expression in a human cell. In some embodiments, the expression vector is configured for expression in a lymphocyte.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, electroporation, viral infection, or direct alteration of the cell's genome. In some embodiments, the gene is an open reading frame. In some embodiments, the open reading frame encodes the chimeric polypeptide of the invention. In some embodiments, the open reading frame is in an expression vector such as plasmid or viral vector. Expression vectors are well known in the art and are available commercially from companies such as Addgene, Sigma Aldrich, Genscript and many others.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

In some embodiments, the vector is a viral vector. The vector may be a DNA plasmid delivered via non-viral methods or via viral methods. The viral vector may be a retroviral vector, a herpesviral vector, an adenoviral vector, an adeno-associated viral vector or a poxviral vector. The promoter may be active in mammalian cells. The promoters may be active in a human cell. The promoter may be active in a lymphocyte.

In some embodiments, the open reading frame is operably linked to at least one regulatory element. In some embodiments, the regulatory element is a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the nucleic acid sequence encoding human Granzyme B is provided by accession number NM_004131, NM_001346011, or NR_144343. In some embodiments, the nucleic acid sequence encoding human Granzyme B is provided by accession number NM_004131 or NM_001346011. In some embodiments, the nucleic acid sequence encoding human Granzyme B is provided by accession number NM_004131. In some embodiments, human Granzyme B is encoded by the nucleotide sequence agctccaaccagggcagccttcctgagaagatgcaaccaatcctgcttctgctggccttcctcctgctgcccagggcagatgcagg ggagatcatcgggggacatgaggccaagccccactcccgcccctacatggcttatcttatgatctgggatcagaagtctctgaaga ggtgcggtggcttcctgatacgagacgacttcgtgctgacagctgctcactgttggggaagctccataaatgtcaccttgggggcc cacaatatcaaagaacaggagccgacccagcagtttatccctgtgaaaagacccatcccccatccagcctataatcctaagaactt ctccaacgacatcatgctactgcagctggagagaaaggccaagcggaccagagctgtgcagcccctcaggctacctagcaaca aggcccaggtgaagccagggcagacatgcagtgtggccggctgggggcagacggcccccctgggaaaacactcacacacac tacaagaggtgaagatgacagtgcaggaagatcgaaagtgcgaatctgacttacgccattattacgacagtaccattgagttgtgc gtgggggacccagagattaaaaagacttcctttaagggggactctggaggccctcttgtgtgtaacaaggtggcccagggcattgt ctcctatggacgaaacaatggcatgcctccacgagcctgcaccaaagtctcaagctttgtacactggataaagaaaaccatgaaac gctactaactacaggaagcaaactaagcccccgctgtaatgaaacaccttctctggagccaagtccagatttacactgggagaggt gccagcaactgaataaatacctcttagctgagtgga (SEQ ID NO: 15). In some embodiments, SEQ ID NO: 15 encodes SEQ ID NO: 8. It will be understood that modification of SEQ ID NO: 15 can be made in order to modify SEQ ID NO: 8. For example, to remove the first 18 amino acids of SEQ ID NO: 8 one can remove the first 54 nucleotides of SEQ ID NO: 15. If an ATG is removed it can be added back to the beginning of the open reading frame.

In some embodiments, the nucleic acid sequence encoding mouse Granzyme B is provided by accession number NM_013542. In some embodiments, mouse Granzyme B is encoded by the nucleotide sequence ATGAAGATCCTCCTGCTACTGCTGACCTTGTCTCTGGCCTCCAGGACAAAGGCA GGGGAGATCATCGGGGGACATGAAGTCAAGCCCCACTCTCGACCCTACATGGC CTTACTTTCGATCAAGGATCAGCAGCCTGAGGCGATATGTGGGGGCTTCCTTAT TCGAGAGGACTTTGTGCTGACTGCTGCTCACTGTGAAGGAAGTATAATAAATG TCACTTTGGGGGCCCACAACATCAAAGAACAGGAGAAGACCCAGCAAGTCAT CCCTATGGTAAAATGCATTCCCCACCCAGACTATAATCCTAAGACATTCTCCAA TGACATCATGCTGCTAAAGCTGAAGAGTAAGGCCAAGAGGACTAGAGCTGTG AGGCCCCTCAACCTGCCCAGGCGCAATGTCAATGTGAAGCCAGGAGATGTGTG CTATGTGGCTGGTTGGGGAAGGATGGCCCCAATGGGCAAATACTCAAACACGC TACAAGAGGTTGAGCTGACAGTACAGAAGGATCGGGAGTGTGAGTCCTACTTT AAAAATCGTTACAACAAAACCAATCAGATATGTGCGGGGGACCCAAAGACCA AACGTGCTTCCTTTCGGGGGGATTCTGGAGGCCCGCTTGTGTGTAAAAAAGTG GCTGCAGGCATAGTTTCCTATGGATATAAGGATGGTTCACCTCCACGTGCTTTC ACCAAAGTCTCGAGTTTCTTATCCTGGATAAAGAAAACAATGAAAAGCAGC (SEQ ID NO: 17). In some embodiments, SEQ ID NO: 17 encodes SEQ ID NO: 16. It will be understood that modification of SEQ ID NO: 17 can be made in order to modify SEQ ID NO: 16. For example, to remove the first 18 amino acids of SEQ ID NO: 17 one can remove the first 54 nucleotides of SEQ ID NO: 16. If an ATG is removed it can be added back to the beginning of the open reading frame.

In some embodiments, the nucleotide sequence encoding mutant, non-activatable human Granzyme B is encoded by the sequence atgcaaccaatcctgcttctgctggccttcctcctgctgcccagggcagatgcagcagcaatcatcgggggacatgaggccaagc cccactcccgcccctacatggcttatcttatgatctgggatcagaagtctctgaagaggtgcggtggcttcctgatacgagacgactt cgtgctgacagctgctcactgttggggaagctccataaatgtcaccttgggggcccacaatatcaaagaacaggagccgacccag cagtttatccctgtgaaaagacccatcccccatccagcctataatcctaagaacttctccaacgacatcatgctactgcagctggaga gaaaggccaagcggaccagagctgtgcagcccctcaggctacctagcaacaaggcccaggtgaagccagggcagacatgca gtgtggccggctgggggcagacggcccccctgggaaaacactcacacacactacaagaggtgaagatgacagtgcaggaaga tcgaaagtgcgaatctgacttacgccattattacgacagtaccattgagttgtgcgtgggggacccagagattaaaaagacttccttt aagggggactctggaggccctcttgtgtgtaacaaggtggcccagggcattgtctcctatggacgaaacaatggcatgcctccac gagcctgcaccaaagtctcaagctttgtacactggataaagaaaaccatgaaacgcta (SEQ ID NO: 18). In some embodiments, the nucleotide sequence encoding mutant, non-activatable mouse Granzyme B is encoded by the sequence atgaagatcctcctgctactgctgaccttgtctctggcctccaggacaaaggcagcagcaatcatcgggggacatgaagtcaagcc ccactctcgaccctacatggccttactttcgatcaaggatcagcagcctgaggcgatatgtgggggcttccttattcgagaggacttt gtgctgactgctgctcactgtgaaggaagtataataaatgtcactttgggggcccacaacatcaaagaacaggagaagacccagc aagtcatccctatggtaaaatgcattccccacccagactataatcctaagacattctccaatgacatcatgctgctaaagctgaagagt aaggccaagaggactagagctgtgaggcccctcaacctgcccaggcgcaatgtcaatgtgaagccaggagatgtgtgctatgtg gctggttggggaaggatggccccaatgggcaaatactcaaacacgctacaagaggttgagctgacagtacagaaggatcggga gtgtgagtcctactttaaaaatcgttacaacaaaaccaatcagatatgtgcgggggacccaaagaccaaacgtgcttcctttcgggg ggattctggaggcccgcttgtgtgtaaaaaagtggctgcaggcatagtttcctatggatataaggatggttcacctccacgtgctttca ccaaagtctcgagtttcttatcctggataaagaaaacaatgaaaagcag (SEQ ID NO: 41).

In some embodiments, the nucleotide sequence encoding Streptococcus pyogenes Cas9 (SpCas9) is atggacaagaagtatagcatcggcctggatatcggcacaaactccgtgggctgggccgtgatcaccgacgagtacaaggtgcca agcaagaagtttaaggtgctgggcaacaccgatagacactccatcaagaagaatctgatcggcgccctgctgttcgactctggcg agacagccgaggccacacggctgaagagaaccgcccggagaaggtatacacgccggaagaataggatctgctacctgcagga gatcttcagcaacgagatggccaaggtggacgattctttctttcaccgcctggaggagagcttcctggtggaggaggataagaagc acgagcggcaccctatctttggcaacatcgtggacgaggtggcctatcacgagaagtacccaacaatctatcacctgaggaagaa gctggtggactccaccgataaggccgacctgcgcctgatctatctggccctggcccacatgatcaagttccggggccactttctgat cgagggcgatctgaacccagacaatagcgatgtggacaagctgttcatccagctggtgcagacctacaatcagctgtttgaggag aaccccatcaatgcctctggagtggacgcaaaggcaatcctgagcgccagactgtccaagtctagaaggctggagaacctgatc gcccagctgccaggcgagaagaagaacggcctgtttggcaatctgatcgccctgtccctgggcctgacacccaacttcaagtcta attttgatctggccgaggacgccaagctgcagctgtccaaggacacctatgacgatgacctggataacctgctggcccagatcgg cgatcagtacgccgacctgttcctggccgccaagaatctgtctgacgccatcctgctgagcgatatcctgcgcgtgaacaccgaga tcacaaaggcccccctgagcgcctccatgatcaagagatatgacgagcaccaccaggatctgaccctgctgaaggccctggtga ggcagcagctgcctgagaagtacaaggagatcttctttgatcagagcaagaatggatacgcaggatatatcgacggaggagcatc ccaggaggagttctacaagtttatcaagcctatcctggagaagatggacggcacagaggagctgctggtgaagctgaatcggga ggacctgctgaggaagcagcgcacctttgataacggcagcatccctcaccagatccacctgggagagctgcacgcaatcctgcg ccggcaggaggacttctacccatttctgaaggataaccgggagaagatcgagaagatcctgacattcagaatcccctactatgtgg gacctctggcccggggcaatagcagatttgcctggatgacccgcaagtccgaggagacaatcacaccctggaacttcgaggagg tggtggataagggcgcctctgcccagagcttcatcgagcggatgaccaattttgacaagaacctgcctaatgagaaggtgctgcca aagcactctctgctgtacgagtatttcaccgtgtataacgagctgacaaaggtgaagtacgtgaccgagggcatgagaaagcctgc cttcctgagcggcgagcagaagaaggccatcgtggacctgctgtttaagaccaataggaaggtgacagtgaagcagctgaagga ggactatttcaagaagatcgagtgttttgattctgtggagatcagcggcgtggaggacaggtttaacgcctccctgggcacctacca cgatctgctgaagatcatcaaggataaggacttcctggacaacgaggagaatgaggatatcctggaggacatcgtgctgaccctg acactgtttgaggatagggagatgatcgaggagcgcctgaagacatatgcccacctgttcgatgacaaagtgatgaagcagctga agagaaggcgctacaccggatggggccggctgagcagaaagctgatcaatggcatccgcgacaagcagtctggcaagacaat cctggactttctgaagagcgatggcttcgccaaccggaacttcatgcagctgatccacgatgactccctgaccttcaaggaggatat ccagaaggcacaggtgtctggacagggcgacagcctgcacgagcacatcgccaacctggccggctctcctgccatcaagaagg gcatcctgcagaccgtgaaggtggtggacgagctggtgaaagtgatgggcaggcacaagccagagaacatcgtgatcgagatg gcccgcgagaatcagaccacacagaagggccagaagaactcccgggagagaatgaagagaatcgaggagggcatcaagga gctgggctctcagatcctgaaggagcaccccgtggagaacacacagctgcagaatgagaagctgtatctgtactatctgcagaat ggccgggatatgtacgtggaccaggagctggatatcaacagactgtctgattatgacgtggatcacatcgtgccacagtccttcctg aaggatgactctatcgacaataaggtgctgaccaggagcgacaagaaccgcggcaagtccgataatgtgccctctgaggaggtg gtgaagaagatgaagaactactggaggcagctgctgaatgccaagctgatcacacagaggaagtttgataacctgaccaaggca gagaggggaggactgtccgagctggacaaggccggcttcatcaagcggcagctggtggagacaagacagatcacaaagcacg tggcccagatcctggattctagaatgaacacaaagtacgatgagaatgacaagctgatcagggaggtgaaagtgatcaccctgaa gtccaagctggtgtctgactttaggaaggatttccagttttataaggtgcgcgagatcaacaattatcaccacgcccacgacgcctac ctgaacgccgtggtgggcacagccctgatcaagaagtaccctaagctggagtccgagttcgtgtacggcgactataaggtgtacg atgtgcgcaagatgatcgccaagtctgagcaggagatcggcaaggccaccgccaagtatttcttttacagcaacatcatgaatttctt taagaccgagatcacactggccaatggcgagatcaggaagcgcccactgatcgagacaaacggcgagacaggcgagatcgtg tgggacaagggcagggattttgccaccgtgcgcaaggtgctgagcatgccccaagtgaatatcgtgaagaagaccgaggtgca gacaggcggcttctccaaggagtctatcctgcctaagcggaactccgataagctgatcgccagaaagaaggactgggaccccaa gaagtatggcggcttcgacagccctacagtggcctactccgtgctggtggtggccaaggtggagaagggcaagagcaagaagc tgaagtccgtgaaggagctgctgggcatcaccatcatggagcgcagctccttcgagaagaatcctatcgactttctggaggccaag ggctataaggaggtgaagaaggacctgatcatcaagctgccaaagtactctctgtttgagctggagaacggaaggaagagaatgc tggcaagcgccggagagctgcagaagggcaatgagctggccctgccctccaagtacgtgaacttcctgtatctggcctcccacta cgagaagctgaagggctctcctgaggataacgagcagaagcagctgtttgtggagcagcacaagcactatctggacgagatcat cgagcagatcagcgagttctccaagagagtgatcctggccgacgccaatctggataaggtgctgtccgcctacaacaagcaccg ggataagccaatcagagagcaggccgagaatatcatccacctgtttaccctgacaaacctgggagcaccagcagccttcaagtatt ttgacaccacaatcgacaggaagcggtacaccagcacaaaggaggtgctggacgccacactgatccaccagtccatcaccggc ctgtacgagacacggatcgacctgtctcagctgggaggcgat (SEQ ID NO: 42).

In some embodiments, a plasmid encoding human Granzyme B comprising mutation of the GE dipeptide to AA and linked to crmCherry is provided in SEQ ID NO: 29. A schematic of the coding region of this plasmid is presented as the first row (labeled 1_2) of FIG. 1. In some embodiments, a plasmid encoding human Granzyme B linked to crmCherry is provided in SEQ ID NO: 30. A schematic of the coding region of this plasmid is presented as the second row (labeled 13) of FIG. 1. In some embodiments, a plasmid encoding human Granzyme B comprising mutation of the GE dipeptide to AA and linked to full-length Cas9 with an N-terminal NLS followed by P2A peptide-crmCherry is provided in SEQ ID NO: 31. A schematic of the coding region of this plasmid is presented as the third row (labeled 1_4) of FIG. 1. In some embodiments, a plasmid encoding human Granzyme B comprising mutation of the GE dipeptide to AA but lacking the ER signal peptide and linked to full-length Cas9 with an N-terminal NLS followed by P2A peptide-crmCherry is provided in SEQ ID NO: 66. A schematic of the coding region of this plasmid is presented as the seventh row (labeled 1_16) of FIG. 1. In some embodiments, a plasmid encoding human Granzyme B and linked to full-length Cas9 with an N-terminal NLS followed by a P2A-crmCherry is provided in SEQ ID NO: 32. A schematic of the coding region of this plasmid is presented as the fourth row (labeled 1_5) of FIG. 1. In some embodiments, a plasmid encoding human Granzyme B lacking the ER signal peptide and linked to full-length Cas9 with an N-terminal NLS followed by a P2A-crmCherry is provided in SEQ ID NO: 65. A schematic of the coding region of this plasmid is presented as the eighth row (labeled 1_15) of FIG. 1. In some embodiments, a plasmid encoding human Granzyme B comprising mutation of the GE dipeptide to AA and linked to full-length Cas9 with an N-terminal NLS by the cleavable linker denoted by SEQ ID NO: 2 and followed by P2A-crmCherry is provided in SEQ ID NO: 33. A schematic of the coding region of this plasmid is presented as the fifth row (labeled 1_6) of FIG. 1. In some embodiments, a plasmid encoding human Granzyme B comprising mutation of the GE dipeptide to AA but lacking the ER signal peptide and linked to full-length Cas9 with an N-terminal NLS by the cleavable linker denoted by SEQ ID NO: 2 and followed by P2A-crmCherry is provided in SEQ ID NO: 67. A schematic of the coding region of this plasmid is presented as the ninth row (labeled 1_17) of FIG. 1. In some embodiments, a plasmid encoding human wild-type Granzyme B linked to full-length Cas9 with an N-terminal NLS by the cleavable linker denoted by SEQ ID NO: 2 and then followed by a P2A-crmCherry is provided in SEQ ID NO: 34. A schematic of the coding region of this plasmid is presented as the sixth row (labeled 17) of FIG. 1. In some embodiments, a plasmid encoding human wild-type Granzyme B lacking the ER signal peptide linked to full-length Cas9 with an N-terminal NLS by the cleavable linker denoted by SEQ ID NO: 2 and then followed by a P2A-crmCherry is provided in SEQ ID NO: 68. A schematic of the coding region of this plasmid is presented as the tenth row (labeled 1_18) of FIG. 1. In some embodiments, a plasmid encoding mouse wild-type Granzyme B linked to crmCherry is provided in SEQ ID NO: 35. In some embodiments, a plasmid encoding mouse Granzyme B comprising mutation of the GE dipeptide to AA and linked to crmCherry is provided in SEQ ID NO: 36. In some embodiments, a plasmid encoding mouse wild-type Granzyme B and linked to full-length Cas9 with an N-terminal NLS followed by a P2A peptide and then crmCherry is provided in SEQ ID NO: 37. In some embodiments, a plasmid encoding mouse Granzyme B comprising mutation of the GE dipeptide to AA and linked to full-length Cas9 with an N-terminal NLS and then followed by a P2A peptide and then crmCherry is provided in SEQ ID NO: 38. In some embodiments, a plasmid encoding mouse wild-type Granzyme B and linked to full-length Cas9 with an N-terminal NLS by a cleavable linker and followed by a P2A peptide and then crmCherry is provided in SEQ ID NO: 39. In some embodiments, a plasmid encoding mouse Granzyme B comprising mutation of the GE dipeptide to AA and linked to full-length Cas9 with an N-terminal NLS by a cleavable linker and then followed by a P2A peptide and then crmCherry is provided in SEQ ID NO: 40.

In some embodiments, a plasmid encoding human Granzyme B comprising mutation of the GE dipeptide to AA and linked to a meganuclease with an N-terminal NLS is provided in SEQ ID NO: 69. A schematic of the coding region of this plasmid is presented as the eleventh row (labeled 2_4) of FIG. 1. In some embodiments, a plasmid encoding human Granzyme B comprising mutation of the GE dipeptide to AA but lacking the ER signal peptide and linked to a meganuclease with an N-terminal NLS is provided in SEQ ID NO: 70. A schematic of the coding region of this plasmid is presented as the twelfth row (labeled 2_5) of FIG. 1. In some embodiments, a plasmid encoding human Granzyme B comprising mutation of the GE dipeptide to AA and linked to a meganuclease with an N-terminal NLS followed by GFP is provided in SEQ ID NO: 71. A schematic of the coding region of this plasmid is presented as the thirteenth row (labeled 2_6) of FIG. 1. In some embodiments, a plasmid encoding a meganuclease with an N-terminal NLS is provided in SEQ ID NO: 5. In some embodiments, a plasmid encoding a meganuclease with an N-terminal NLS and a C-terminal NLS is provided in SEQ ID NO: 6.

Modified Cells

By another aspect, there is provided a modified cell comprising a therapeutic agent.

In some embodiments, the cell is a cell that expresses Granzyme. In some embodiments, the cell naturally expresses granzyme. In some embodiments, the cell expresses granzyme before modification. In some embodiments, the Granzyme is Granzyme B. In some embodiments, the cell is a cell that expresses Perforin. In some embodiments, the cell expresses perforin before the modification. In some embodiments, the cell is a cell that expresses Granzyme and Perforin. In some embodiments, expression is secretion. Cells that express/secrete the combination of Granzyme and Perforin are well known in the art and any such cells may be used. In some embodiments, the cell is an immune cell. In some embodiments the cell is a white blood cell. In some embodiments, the cell is a leukocyte. In some embodiments, a leukocyte is a lymphocyte or a myeloid cell. In some embodiments, the cell is a CD45+ cell. In some embodiments, the white blood cell (CD45+ cell) expresses Granzyme and/or Perforin. In some embodiments, the cell is capable of forming an immune synapse. In some embodiments, the cell is a cell that produces immune synapses. In some embodiments, the cell is characterized by the ability to form an immune synapse with a target cell. In some embodiments, the cell is a lymphocyte. In some embodiments, the cell is a myeloid cell. In some embodiments, the cell is selected from a lymphocyte and a myeloid cell. In some embodiments, the myeloid cell is a macrophage. In some embodiments, myeloid cells comprise macrophages. In some embodiments, the myeloid cell is a dendritic cell. In some embodiments, myeloid cells comprise dendritic cells. In some embodiments, the lymphocyte is a T cell. In some embodiments, the lymphocyte is an NK cell. In some embodiments, the lymphocyte is selected from a T cell and an NK cell. In some embodiments, the cell is selected from a T cell, an NK cell and a myeloid cell. In some embodiments, the cell is selected from a T cell, an NK cell and a macrophage. In some embodiments, a cell that can form an immune synapse is selected from a T cell, an NK cell, a B cell, a mast cell, a neutrophil and a macrophage. In some embodiments, a cell that can form an immune synapse is selected from a T cell, an NK cell, a B cell, a mast cell, a neutrophil and a myeloid cell. In some embodiments, a cell that can form an immune synapse is selected from a T cell, an NK cell, a B cell, a mast cell and a macrophage. a cell that can form an immune synapse is selected from a T cell, an NK cell, a B cell, and a macrophage. In some embodiments, a cell that can form an immune synapse is selected from a T cell, an NK cell, a B cell, a mast cell and a macrophage. a cell that can form an immune synapse is selected from a T cell, an NK cell, a B cell, and a myeloid cell. In some embodiments, a cell that can form an immune synapse is selected from a T cell, an NK cell, and a macrophage. In some embodiments, a cell that can form an immune synapse is selected from a T cell, an NK cell, and a myeloid cell. In some embodiments, the cell is selected from a lymphocyte and a cell that is capable of producing a phagocytic synapse. In some embodiments, an immune synapse is a phagocytic synapse. In some embodiments, the cell is selected from a lymphocyte and a macrophage. In some embodiments, the cell is selected from a lymphocyte and a myeloid cell.

As used herein, the term “immune synapse” and “immunological synapse” are used interchangeably are refer to a physical interface between an immune cell and a target cell, which is often an antigen-providing cell. In some embodiments, an immune synapse is a supramolecular activation cluster. In some embodiments, the synapse comprises three concentric rings of protein clusters that mediate transfer of proteins between the immune cell and its target. In some embodiments, the synapse comprises adhesion molecules in the periphery. It will be understood by a skilled artisan that the adhesion molecules of the immunological synapse result in increased avidity and sustained contact between effector and targe cell. This increased avidity/contact enables more potent transport of molecules. In this way synapse formation produces a solution to the problem of successful and efficient transfer of therapeutic molecules. In some embodiments, the synapse comprises a high density of T cell receptors and co-stimulatory molecules. In some embodiments, the high density is in the center of the immunological synapse. In some embodiments, immune synapse formation triggers immune cell activation. In some embodiments, immune synapse formation triggers lymphocyte activation. In some embodiments, immune synapse formation triggers T cell activation. In some embodiments, activation is activation above a minimum threshold needed for execution of effector function. In some embodiments, activation is activation above or equal to artificial activation by an anti-CD3 antibody. In some embodiments, activation is activation above or equal to artificial activation by IL-2. In some embodiments, activation is activation above artificial activation by an anti-CD3 antibody. In some embodiments, activation is activation above artificial activation by IL-2.

In some embodiments, the cell is a cell of adoptive cell transfer. In some embodiments, the cell is a therapeutic cell. In some embodiments, the cell is a tumor infiltrating lymphocyte (TIL). In some embodiments, the cell is an adoptive T cell. In some embodiments, the cell is an adoptive NK cell. In some embodiments, the cell is a CAR cell. In some embodiments, the CAR cell is a CAR T cell. In some embodiments, the CAR cell is a CAR NK cell. In some embodiments, the cell is a cell in culture. In some embodiments, the cell has been expanded. In some embodiments, the cell is in vivo.

In some embodiments, the cell is modified to express the therapeutic agent and/or molecule and/or protein. In some embodiment, the modification is performed in-vitro or ex-vivo. In some embodiments, the modification is performed by introduction of a protein of interest or a ribonucleoprotein of interest or a nucleotide sequence of interest or other molecule of interest. In vitro introduction of molecule of interest to cells is well known in the art and may be performed for a non-limiting example by electroporation, transfection, infection, by plasmid, virus and liposomes. In some embodiment the modification is performed by introducing a nucleotide sequence and/or molecule (e.g., DNA and RNA) to be expressed in the lymphocyte.

In some embodiments, the cell is a mammalian cell. In some embodiments, the cell is a human cell. In some embodiments, the cell is a primary cell. In some embodiments, the cell is a peripheral blood cell. In some embodiments, the lymphocyte is a peripheral blood lymphocyte. In some embodiments, the peripheral blood cell is a peripheral blood mononuclear cell (PBMC). In some embodiments, the cell is a bone marrow cell. In some embodiments, the lymphocyte is a bone marrow lymphocyte. In some embodiments, the lymphocytes are tissue resident lymphocytes. In some embodiments, the cell is a cell in culture. In some embodiments, the cell is an ex vivo cell. In some embodiments, the cell is from a subject. In some embodiments, the cell is in a subject. In some embodiments, the cell is from a cell line. In some embodiments, the cell line is a granzyme knockdown cell line.

In some embodiments, the cell is a naturally-occurring cell. In some embodiments, the cell is a differentiated from another cell in culture. In some embodiments, the differentiation is trans-differentiation. In some embodiments, the differentiation is a naturally-occurring differentiation. Methods of producing cells in culture by differentiation are well known in the art and any such method may be used to produce the cells used to produce the modified cells of the invention. In some embodiments, stem cells are differentiated to produce the cells. In some embodiments, the stem cells are hematopoietic stem cells (HSCs). In some embodiments, the stem cells are embryonic stem cells (ESCs). In some embodiments, the stem cells are not ESCs. In some embodiments, the cells are not derived from embryonic cells. In some embodiments, the stem cells are multipotent stem cells. In some embodiments, the stem cells are pluripotent stem cells. In some embodiments, the stem cells are induced pluripotent stem cells (iPSCs). In some embodiments, the stem cells are mesenchymal stromal cells or mesenchymal stem cells (MSCs). In some embodiments, the cells are not derived or trans-differentiated from MSCs.

In some embodiments, the cell comprises secretory lysosomes. In some embodiments, the cell comprises lytic granules. In some embodiments, the cell is a lytic cell. In some embodiments, the lymphocyte comprises lytic granules. In some embodiments, the lymphocyte expresses lytic granules in response to activation. In some embodiments, the lytic granules become polarized in response to activation. In some embodiments, the lymphocyte comprises polarized lytic granules. In some embodiments, the cell expresses at least one lymphocyte lytic granule-secreted protein. In some embodiments, the lymphocyte expresses at least one lymphocyte lytic granule-secreted protein. In some embodiments, the expression is in response to activation. In some embodiments, the lymphocyte is a T cell. In some embodiments, the lymphocyte is a B cell. In some embodiments, the lymphocyte is an NK cell. In some embodiments, the lymphocyte is a naive lymphocyte. In some embodiments, the lymphocyte is an activated lymphocyte. Activation of lymphocytes is well known in the art and can be done by any known method including those disclosed hereinbelow. Such methods include anti-CD3 stimulation and/or IL-2 stimulation. In some embodiments, the T cell is a CD8 positive T cell. In some embodiments, the T cell is a cytotoxic T lymphocyte. In some embodiments, the T cell has been modified to inhibit its cytotoxicity. In some embodiments, the T cell has been modified to abolish its cytotoxicity. In some embodiments, the T cell is a helper T cell. In some embodiments, the T cell is a CD4 positive T cell. In some embodiments, the T cell is an aβ T-cell. In some embodiments, the T cell is a γδ T-cell. In some embodiments, the T cell is a regulatory T cell. In some embodiments, the lymphocyte is an NK cell. In some embodiments, the lymphocyte is selected from a T cell and an NK cell. In some embodiments, the NK cell is a natural killer T (NKT) cell.

In some embodiments, the cell is a non-cytotoxic cell. In some embodiments, the cell is a non-lytic cell. In some embodiments, the cell does not induce lysis in a target cell. In some embodiments, the cell does not induce apoptosis in a target cell. In some embodiments, the lymphocyte is a non-cytotoxic lymphocyte. In some embodiments, the cell is non-cytotoxic before modification. In some embodiments, the lymphocyte is non-cytotoxic before modification. In some embodiments, the cell is naturally cytotoxic and is modified to reduce cytotoxicity. In some embodiments, the cell is naturally cytotoxic and is modified to inhibit cytotoxicity. In some embodiments, the cell is naturally cytotoxic and is modified to abolish cytotoxicity. In some embodiments, the modification is mutation of the TCR. In some embodiments, the modification is removal of the TCR. In some embodiments, the modification is down-regulation of the TCR. In some embodiments, the modification is inhibition of expression of the TCR. In some embodiments, the TCR is the endogenous TCR. In some embodiments, the modification is mutation of a granzyme. In some embodiments, the cell is a cell of a granzyme knockdown cell line. In some embodiments, the knockdown is CRISPR removal. In some embodiments, the modification is mutation of a proinflammatory cytokine. In some embodiments, the cell is a cell of a proinflammatory cytokine knockdown cell line. In some embodiments, cytotoxicity is endogenous cytotoxicity. In some embodiments, the cell is not cytotoxic, and the chimeric molecule is cytotoxic. In some embodiments, the cell is not cytotoxic, and the molecule of interest is cytotoxic. In some embodiments, the cell comprises mutation or knockout of a cytotoxic protein. In some embodiments, the cytotoxic protein is a cytotoxic protein other than a lytic granule-secreted protein. In some embodiments, the knockout is by genetic ablation of the locus encoding the cytotoxic protein. In some embodiments, the genetic ablation is by a genome-editing protein. In some embodiments, the genetic ablation is by CRISPR.

In some embodiments, the modified cells are further modified/engineered to prevent lytic effect on target cells upon/following activation. In some embodiments, activation is activation of the granzyme-perforin pathway. In some embodiments, the modified lymphocytes are engineered to knockdown endogenous expression of a lymphocytes lytic-granule-secreted protein. In some embodiments, knockdown is knockout. In some embodiments, the modified cells are engineered to knockdown a gene encoding for cell-mediating killing elements. Example of such killing elements include, but are not limited to endogenous granzyme, FasL and Trail. In some embodiments, endogenous expression of FasL is knocked down. In some embodiments, endogenous expression of a Trail receptor is knocked down. In some embodiments, endogenous expression of a proinflammatory cytokine is knocked down. In some embodiments, endogenous expression of a proinflammatory cytokine is inhibited. Proinflammatory cytokines are well known in the art and include, for non-limiting example, TNFa and INFg. In some embodiments, knocked down is knocked out. Genetic knockdown/knockout can be accomplished by any convenient method known in the art, such as genome-editing, for example with CRISPR/Cas9 or introduction of sequences encoding a specific siRNA, shRNA, miRNA or similar inhibitory nucleic acid molecules. In some embodiments, the further modification/engineering is performed in vitro. In some embodiments, the further modification/engineering is performed ex vivo.

In some embodiments, the cell comprises a therapeutic agent and a targeting moiety. In some embodiments, the targeting moiety is a separate molecule from the therapeutic agent. In some embodiments, the therapeutic agent is not a targeting molecule. In some embodiments, the targeting moiety is an engineered molecule. In some embodiments, the targeting moiety is non-naturally occurring. In some embodiments, the targeting moiety is an engineered TCR. In some embodiments, the targeting moiety is a chimeric antigen receptor (CAR). In some embodiments, the targeting moiety activates the lymphocyte upon binding its target. In some embodiments, the targeting moiety initiates an activation cascade upon binding its target. In some embodiments, the targeting moiety comprises a target-binding domain. In some embodiments, the targeting moiety comprises an activation domain. In some embodiments, the targeting moiety is a transmembrane protein. In some embodiments, the targeting moiety comprises a co-activation domain. In some embodiments, the lymphocyte is a CAR-T cell. In some embodiments, the lymphocyte is a CAR-NK cell. In some embodiments, the lymphocyte is a TIL.

As used herein, the terms “CAR-T cell” and “CAR-NK cell” refer to an engineered receptor which has specificity for at least one target protein of interest and is grafted onto an immune cell (a lymphocyte). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell. It will be understood by a skilled artisan that if the cell of the invention has been rendered non-cytotoxic or is naturally non-cytotoxic, activation will lead to secretion of vesicles containing the therapeutic agent or molecule of interest but will still not lead to killing of the target cell. In some embodiments, the target cell is an engaged cell. In some embodiments, engaged is engaged by the CAR.

CAR-T and CAR-NK cells and their vectors are well known in the art. Such cells target and engage the protein for which the receptor binds. In some embodiments, a CAR-T or CAR-NK cell targets at least one viral protein. In some embodiments, a CAR-T or CAR-NK cell targets at least one cancer protein. In some embodiments, a CAR-T or CAR-NK cell targets at least one surface protein. In some embodiments, the surface protein is a target protein. In some embodiments, the surface protein is on the surface of a target cell.

Construction of CAR-T cells is well known in the art. In one non-limiting example, a monoclonal antibody to a viral protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z, CD27, CD28, 4-1BB, OX40, CD30, CD40, PD-1, ICOS, lymphocyte function-associated antigen-1 (LFA-1), CD2, CD 7, LIGHT, NKG2C, B7- H3, and a ligand that specifically binds with CD83. In some embodiments, the vector also comprises a CD3Z signaling domain. This vector is then transfected, for example by lentiviral infection, or electroporated into a lymphocyte.

In some embodiments, the cell is from a subject. In some embodiments, the cell is autologous to the subject. In some embodiments, the cell is allogeneic to the subject. In some embodiments, the cell is a universal allogeneic lymphocyte. In some embodiments, the cell is a non-immunogenic lymphocyte. In some embodiments, the cell is an off-the-shelf lymphocyte. In some embodiments, the cell is syngeneic to the subject. In some embodiments, the cells share a matched HLA type to the subject. Autologous cells refer to cells derived from the same subject to which they are re-introduced following modification (e.g., ex vivo/in vitro modification. The cells may be extracted from the patient's blood by Leukapheresis. Methods of cell/lymphocyte extraction are well known in the art and any such method may be employed. In some embodiments, the cells are for adoptive cell transfer. In some embodiments, the lymphocytes are for adoptive cell transplant.

In some embodiments, the therapeutic agent is the molecule of interest. In some embodiments, the therapeutic agent is the protein of interest. In some embodiments, the therapeutic agent is the chimeric molecule. In some embodiments, the therapeutic agent is the chimeric polypeptide. In some embodiments, the therapeutic agent is a protein. In some embodiments, the therapeutic agent is a proteinaceous agent. In some embodiments, the therapeutic agent is exogenous to the leukocyte. In some embodiments, the therapeutic agent is not naturally occurring. In some embodiments, the therapeutic agent is not cytotoxic. In some embodiments, the therapeutic agent is not lytic. In some embodiments, the therapeutic agent is a chimeric molecule of the invention. In some embodiments, the therapeutic agent is a chimeric polypeptide of the invention.

In some embodiments, the therapeutic agent acts in the cytoplasm. In some embodiments, the therapeutic agent acts in the nucleus. In some embodiments, the therapeutic agent acts within a cell. In some embodiments, the therapeutic agent's target is an internal target. In some embodiments, the therapeutic agent's target is a nuclear target. In some embodiments, the therapeutic agent's target is a cytoplasmic target. In some embodiments, the therapeutic agent's target is not a cell surface target. In some embodiments, the therapeutic agent's target is not a receptor.

In some embodiments, the therapeutic agent treats a disease, disorder, or condition. In some embodiments, the therapeutic agent treats a disease. Examples of such disease include, but are not limited to a genetic disease, an autoimmune disease, a bacterial disease, a viral disease, an inflammatory disease, a proliferative disease, a cardiovascular disease, a degenerative disease, a brain disease, a digestive disease, a liver disease, a neurological disease and an energy homeostasis disease. In some embodiments, a proliferative disease is cancer. In some embodiments, the disease is not cancer. In some embodiments, the therapeutic agent is not an anti-cancer therapeutic. In some embodiments, the leukocyte comprises a targeting moiety that targets a cell of the disease. It will be understood by a skilled artisan that the cells at the route of a disease can be discerned and in turn treated by lymphocytes of the invention. For example, a neuronal disease can be treated with lymphocytes with a neuron targeting moiety, an energy homeostasis disease can be treated with lymphocytes with a pancreatic targeting moiety, etc. In some embodiments, the therapeutic agent treats a condition. Examples of conditions include, but are not limited to inflammatory conditions, aging-related conditions, degenerative conditions and many others.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, the disease is a genetic disease. In some embodiments, the condition is a genetic condition. In some embodiments, the disorder is a genetic disorder. In some embodiments, a genetic disease, condition or disorder is one that is caused by a genetic mutation. In some embodiments, a disease is a disease condition or disorder. In some embodiments, the disease is not cancer. In some embodiments, the genetic mutation is a somatic mutation. In some embodiments, the genetic mutation is a germ-line mutation. In some embodiments, the genetic disease, disorder or condition is treatable by gene therapy. In some embodiments, the therapeutic agent is a gene editing agent. In some embodiments, the therapeutic agent is a gene editing protein. In some embodiments, the therapeutic agent is a gene editing complex. In some embodiments, the complex is an RNA-protein complex. In some embodiments, gene editing is genome-editing. In some embodiments, the therapeutic agent comprises Cas9 or a homolog, ortholog or variant thereof.

Genetic disease and disorders are well known in the art and treatment of any such disease/disorder is envisioned. Examples of genetic disease and disorders include, but are not limited to: Angelmen syndrome, ankylosing spondylitis, Apert syndrome, congenital adrenal hyperplasia, cystic fibrosis, Down syndrome, fragile X syndrome, haemochromatosis, hemophilia, Huntington's disease, Klinefelter syndrome, Marfan syndrome, muscular dystrophy, neurofibromatosis, Noonan syndrome, Prader-Willi syndrome, Rett syndrome, Tay-Sachs disease, thalassemia, Tourette syndrome, Turner syndrome, Von Willebrand disease and Williams syndrome.

In some embodiments, the therapeutic agent is in a lytic granule of the cell. In some embodiments, the therapeutic agent is in a lytic granule of the lymphocyte. In some embodiments, the therapeutic agent is in a secretory granule of the cell. In some embodiments, the therapeutic agent is in a secretory granule of the lymphocyte. In some embodiments, the therapeutic agent is in a secretory lysosome of the cell. In some embodiments, the therapeutic agent is in a secretory lysosome of the lymphocyte. In some embodiments, the therapeutic agent is not in an exosome of the cell. In some embodiments, a lytic granule of the lymphocyte comprises the therapeutic agent. In some embodiments, a secretory granule of the cell comprises the therapeutic agent. In some embodiments, a secretory granule of the lymphocyte comprises the therapeutic agent. In some embodiments, at least 50% of the therapeutic agent/protein of interest is within the granule/lysosome. In some embodiments, at least 20, 25, 30, 35, 40, 45, 50, 55, 60, 65, 70, 75, 80, 85, 90, 95, 97, 99 or 100% of the therapeutic agent/protein of interest is within the granule/lysosome. Each possibility represents a separate embodiment of the invention. In some embodiments, at least 75% of the therapeutic agent/protein of interest is within the granule/lysosome. It will be understood by a skilled artisan that having the therapeutic agent in the lytic/secretory granule and not in exosomes (or not solely in exosomes) is advantageous. Exosomes are produced indiscriminately, and the therapeutic agent would be continuously released if it were in exosomes. Further, under standard conditions exosomes are not highly produced. In contrast, the lytic/secretory vesicle is only secreted upon cellular activation. Thus, as the lymphocyte travels through the subject before engaging its target the therapeutic agent in the lytic vesicle is not released, though exosomal content may be. Upon engagement of the target cell, the lymphocyte becomes activated and large numbers of lytic vesicles are released. Thus, employing the lytic pathway and not the exosome pathway ensures target delivery and robust delivery at the same time. Advantageously, delivery of therapeutic agents via leukocytes (lymphocytes and myeloids), which are activated by recognition of target cells, will result in increased specificity to target cells or cells within a target tissue compared to other known methods of in vivo delivery.

Pharmaceutical Compositions

By another aspect there is provided a composition comprising a modified cell of the invention.

In some embodiments, the composition is a pharmaceutical composition. In some embodiments, the composition is a therapeutic composition. In some embodiments, the composition is a composition for use in treatment. In some embodiments, the treatment is treatment of a subject in need thereof. In some embodiments, treatment is treatment of a disease, condition or disorder that is treatable by the therapeutic agent.

In some embodiments, the composition comprises a population of modified cells. In some embodiments, the composition comprises a plurality of modified cells. In some embodiments, the composition comprises at least 1, 2, 3, 4, 5, 6, 7, 9, 10, 20, 30, 40, 50, 60, 70, 80, 90, 100, 200, 300, 400, 500, 600, 700, 800, 900 million or 1 billion modified cells. Each possibility represents a separate embodiment of the invention.

In some embodiments, the composition is formulated for systemic administration. In some embodiments, the composition is formulated for administration to a subject. In some embodiments, the composition is formulated for intravenous administration. In some embodiments, the composition is formulated for local administration. In some embodiments, the composition is formulated for administration to a subject. In some embodiments, formulated for administration to a subject is formulated without unknown chemical content. In some embodiments, a composition formulated for administration to a subject is chemically defined. In some embodiments, a composition formulated for administration does not comprise animal serum. In some embodiments, animal serum is non-human animal serum. In some embodiments, the subject is a human. In some embodiments, the subject is afflicted with a disease.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, oral, subcutaneous, intrathecal, intramuscular, or intraperitoneal.

The dosage administered will be dependent upon the age, health, and weight of the recipient, kind of concurrent treatment, if any, frequency of treatment, and the nature of the effect desired.

In some embodiments, the composition comprises a pharmaceutically acceptable excipient, carrier or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is a carrier suitable for administration to a human. In some embodiments, the composition does not comprise non-human proteins. In some embodiments, the composition is devoid of non-human proteins. In some embodiments, the carrier contains media for the cell. In some embodiments, the media is lymphocyte media. In some embodiments, the media is myeloid media. In some embodiments, the media is macrophage media. In some embodiments, the media is chemically defined media. In some embodiments, the media is devoid of animal protein. In some embodiments, the media is devoid of animal serum. In some embodiments, the composition is devoid of animal serum. In some embodiments, the composition is non-immunogenic. In some embodiments, non-immunogenic in non-immunogenic to the subject.

Methods of Use

By one aspect, there is provided a method of delivering a therapeutic agent to a target cell, the method comprising contacting the target cell with a modified cell of the invention, thereby delivering a therapeutic agent to a target cell.

By another aspect, there is provided a method of delivering a therapeutic agent to a target cell, the method comprising contacting the target cell with a composition of the invention, thereby delivering a therapeutic agent to a target cell.

By another aspect, there is provided a method of delivering a therapeutic agent to a target cell, the method comprising contacting the target cell with a modified cell expressing a chimeric molecule of the invention, thereby delivering a therapeutic agent to a target cell.

By another aspect, there is provided a method of delivering a therapeutic agent to a target cell, the method comprising contacting the target cell with a modified cell expressing a polynucleotide of the invention, thereby delivering a therapeutic agent to a target cell.

In some embodiments, the method is an in vivo method. In some embodiments, the method is an in vitro method. In some embodiments, the method is an ex vivo method. In some embodiments, the method is performed ex vivo. In some embodiments, the target cell is in a subject. In some embodiments, the subject is in need of treatment. In some embodiments, treatment is by the therapeutic agent. In some embodiments, treatment is with the therapeutic agent. In some embodiments, the subject is amenable to treatment by the therapeutic agent. In some embodiments, the method is a method of genome-editing. In some embodiments, the method is a method of gene therapy. In some embodiments, the method is a method of editing a genomic locus. In some embodiments, the subject suffers from a disease. In some embodiments, the subject suffers from a disease or condition. In some embodiments, the disease or condition are treatable by the therapeutic agent.

In some embodiments, there is provided a method of genome editing within a target cell, the method comprising contacting the target cell with a modified cell of the invention, thereby delivering a gene editing agent to a target cell, wherein the gene editing agent modifies/edits a gene within the nucleus of the target cell. In some embodiments, genome editing comprises modifying a gene within a nucleus of the target cell. In some embodiments, the gene editing agent is a gene editing protein. In some embodiments, the gene editing agent modifies a gene within a nucleus of the target cell.

In some embodiments, the method of genome editing is a method of treating a genetic disease. In some embodiments, the method of genome editing further comprises providing a genome editing targeting nucleic acid molecule. In some embodiments, the nucleic acid is an RNA. In some embodiments, the RNA is a guide RNA. In some embodiments, the guide RNA is an sgRNA. In some embodiments, the targeting nucleic acid molecule is compatible with the genome editing protein. Targeting nucleic acid molecules for use with genome editing proteins (such as CAS9) are well known in the art. Methods of designing these sequences are also well known, as are methods for selecting the target sequence for the genome editing. Any such method may be employed. In some embodiments, the nucleic acid molecule is chemically modified. In some embodiments, the nucleic acid molecule comprises a chemically modified backbone.

In some embodiments, the targeting nucleic acid molecule is provided with the genome editing protein. In some embodiments, the genome editing protein and the targeting nucleic acid molecule are provided together. In some embodiments, the genome editing protein and the targeting nucleic acid molecule are provided in an RNP. In some embodiments, the genome editing protein and the targeting nucleic acid molecule are provided separately. In some embodiments, the genome editing protein and the targeting nucleic acid molecule are provided separately to the target cell. In some embodiments, the genome editing protein and the targeting nucleic acid molecule are provided separately to subject.

Methods of nucleic acid molecule delivery are well known in the art and are described herein. Any such method of delivery may be employed. Methods of delivery in vitro such as nucleofection, lipofection, transfection, and viral delivery may be employed. Methods of in vivo delivery such as lentivirus, nanoparticle delivery, microparticle delivery, lipid delivery, ligand conjugated delivery and many more are envisioned. Any method known in the art for nucleic acid delivery many be employed to deliver the targeting nucleic acid to the target cells.

By another aspect, there is provided a method of treating a subject in need thereof, the method comprising administering to the subject a modified cell of the invention, thereby treating a subject.

By another aspect, there is provided a method of treating a subject in need thereof, the method comprising administering to the subject a composition of the invention, thereby treating a subject.

By another aspect, there is provided a method of treating a subject in need thereof, the method comprising administering to the subject a modified cell expressing a chimeric molecule of the invention, thereby treating a subject.

By another aspect, there is provided a method of treating a subject in need thereof, the method comprising administering to the subject a modified cell expressing a polynucleotide of the invention, thereby treating a subject.

By another aspect, there is provided a method of producing a modified cell of the invention, the method comprising providing the cell, and introducing a therapeutic agent into the cell.

By another aspect, there is provided a method of producing a modified cell of the invention, the method comprising providing the cell, and introducing a chimeric polypeptide of the invention into the cell.

In some embodiments, the target cell is a cell of the disease. In some embodiments, the target cell is a cell to which the cell naturally homes. In some embodiments, the target cell is a cell to which lymphocytes naturally home. In some embodiments, the target cell is a cell expressing a surface protein that is a target of a targeting moiety on the cell. In some embodiments, the cell is autologous to the subject. In some embodiments, the cell is allogeneic to the subject. In some embodiments, the cell is syngeneic to the subject. In some embodiments, the cell is an in vitro cell. In some embodiments, the cell is an ex vivo cell. In some embodiments, the cell is in culture. In some embodiments, the cell is in a subject. In some embodiments, the cell is a population of cells expanded in culture.

In some embodiments, the method further comprises obtaining cells. In some embodiments, obtaining is extracting the cells from a subject. In some embodiments, the method further comprises extracting cells from the subject. In some embodiments, the method further comprises obtaining lymphocytes. In some embodiments, obtaining is extracting the lymphocytes from a subject. In some embodiments, the method further comprises extracting lymphocytes from the subject. In some embodiments, the method further comprises expanding the extracted cells. In some embodiments, the method further comprises activating he extracted cells. In some embodiments, the method further comprises activating he extracted lymphocytes. In some embodiments, the method further comprises modifying the extracted cells. In some embodiments, modifying is modifying with a targeting moiety. In some embodiments, modifying is with the therapeutic agent. In some embodiments, modifying is expressing in the cell. In some embodiments, expressing in the cell is modifying. In some embodiments, modifying is with the chimeric molecule of the invention. In some embodiments, modifying is expressing within the cells. In some embodiments, modifying produces modified cells. In some embodiments, modified cells are modified cells of the invention. In some embodiments, the method comprises administering the cells to the subject. In some embodiments, the administering is returning the cells to the subject.

In some embodiments, the modifying or expressing is done after the cells are activated. In some embodiments, the modifying or expressing is done immediately after the activating. In some embodiments, the cells are administered while activated. In some embodiments, the modifying or expressing is done while the cells are active. In some embodiments, the cells are not allowed to return to a stable mode before modifying or expressing. In some embodiments, the modifying or expressing is done in close proximity to the administering. It will be understood by a skilled artisan that for cargo that is transiently loaded it is advantageous not to have the modified cells proliferating for an extended time before administration as this would dilute the concentration of cargo in the effector cells. In some embodiments, the cells are modified not more than 3, 6, 12, 24, 36, 48, 60, 72, 84, 96, 108, or 120 hours before administering. Each possibility represents a separate embodiment of the invention. In some embodiments, the cells are modified not more than 24 hours before administering. In some embodiments, the cells are modified not more than 48 hours before administering. In some embodiments, the cells are modified not more than 72 hours before administering. In some embodiments, the cells are modified not more than 96 hours before administering. In some embodiments, the cells are modified not more than 120 hours before administering. In some embodiments, the modifying or expressing the therapeutic agent is done after the cells are activated. In some embodiments, modifying or expressing a targeting moiety is done before activating. It will be understood by a skilled artisan that in current methods known in the art for expressing cytotoxic proteins/CARs/cytokines and similar molecules in effector cells the effector cells are not activated first. Rather the expression is done first and then the cells are activated. In the instant method the effector cells are first activated. This activation induces the cytoplasm to be full of lytic granules ensuring that the expressed therapeutic agent/chimeric polypeptide will go primarily into the granules and thus facilitate transfer.

In some embodiments, the modified cells are modified to comprise the cargo of interest within granules (secretory lysosomes, such as lytic granules) thereof. In some embodiments, modification after activation results in loading of cargo into granules. In some embodiments, the cargo of interest comprises a fusion protein disclosed hereinabove. In some embodiments, the obtained cells or the modified cells are further engineered to prevent lytic effect on target cells upon/following activation of the granzyme-perforin pathway. In some embodiments, the modified cells are engineered to knock out endogenous expression of the lymphocytes lytic-granule-secreted protein. In some embodiments, the modified cells are engineered to knock out genes encoding for cell-mediating killing elements like endogenous granzyme and/or FasL and/or Trail. In some embodiments, endogenous expression of FasL and Trail receptors, which are typically expressed on T-cell membranes and initiate apoptosis upon binding to their ligands on target cells, may be knocked out. Genetic knockout can be accomplished by any convenient method as known in the art, e.g., editing with CRISPR/Cas9; introduction of sequences encoding a specific siRNA, shRNA, and the like. In some embodiments, the cell is modified to express a targeting nucleic acid molecule.

In some embodiments, delivery is delivery to the cytoplasm of the target cell. In some embodiments, delivery is delivery to the nucleus of the target cell. In some embodiments, delivery is delivery to the cytoplasm or nucleus of a target cell. In some embodiments, delivery is through a perforin pore. In some embodiments, the delivery does not comprise endocytosis by the target cell. In some embodiments, delivery is not by exosomes. In some embodiments, delivery is not by extracellular vesicles. In some embodiments, the extracellular vesicles are not lytic extracellular vesicles. In some embodiments, delivery is not via endosomes in the target cell. In some embodiments, delivery is not via phagocytosis. In some embodiments, delivery is delivery to an immune synapse. In some embodiments, delivery comprises editing a genome of the target cell. In some embodiments, delivery comprises editing a genomic locus of the target cell. In some embodiments, delivery comprises gene therapy. In some embodiments, the method further comprises delivering a targeting nucleic acid molecule to the target cell. In some embodiments, the method further comprises delivering a targeting nucleic acid molecule to the subject.

By another aspect, there is provided a kit comprising a chimeric polypeptide of the invention.

By another aspect, there is provided a kit comprising a polynucleotide of the invention.

By another aspect, there is provided a kit comprising a modified cell of the invention.

By another aspect, there is provided a kit comprising a composition of the invention.

In some embodiments, the kit further comprises a cell. In some embodiments, the kit further comprises means for expressing the polypeptide in a cell. In some embodiments, the method further comprises means for expressing the protein of interest in the cell. In some embodiments, the kit further comprises means for expressing the polynucleotide in a cell. In some embodiments, the kit further comprises instructions for performing a method of the invention.

By another aspect, there is provided a modified cell of the invention, a composition of the invention, a modified cell expressing a chimeric molecule of the invention or a modified cell expressing a polynucleotide of the invention for use in a method of delivering a therapeutic agent to a target cell.

By another aspect, there is provided a modified cell of the invention, a composition of the invention, a modified cell expressing a chimeric molecule of the invention or a modified cell expressing a polynucleotide of the invention for use in a method of treating a subject in need thereof.

In some embodiments, the use comprises a method of delivery/treating comprising administering the modified cell or composition to the target cell or the subject. In some embodiment, the use comprises a method of the invention. In some embodiments, the use comprises a method provided hereinabove. In some embodiments, the use comprises providing the modified cell. In some embodiments, the use comprises extracting the cell from the subject. In some embodiments, the use comprises modifying the cell. In some embodiments, the use comprises returning the cell to the subject. In some embodiments, the use is use in gene therapy.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods

CD8 T cell production and maintenance: Peripheral Blood Mononuclear Cells (PBMC) were isolated from Buffy coats by density gradient centrifugation using Ficoll-Paque. PBMC cells were collected and placed into pre-warmed RPMI 1640 medium (BI, cat #01-100-1A) and then counted and frozen. 20 million cells were frozen per cryopreservation vial.

5 PBMC vials (100 million cells) were defrosted and seeded at a density of 2 million cells per 1 ml of complete medium supplemented with 50 ng/ml of anti-CD3 antibody and 300 U/ml of IL2. Cells were maintained in culture for 5 days. 100 U/ml of IL2 was added in each medium supplement during culture.

CD8 positive cells were isolated by negative selection using CD8 isolation kit (Milteny #130-096-495). The CD8 cells were seeded in cRPMI medium with 100 U/ml of IL2 at a density of 1 million cells/ml. Cells at day 6-10 in culture were used for experiments.

Cell line culture: YTS, P815, and K562 cell lines were maintained in RPMI medium supplemented with 10% Fetal Calf Serum, 1 mM Sodium Pyruvate, 4 mM L-Glutamine, 1% Penicillin-Streptomycin, and 0.1 mM MEM Eagle Non-essential Amino Acids.

MCF-7 cells were maintained in MEM-alpha medium supplemented with 10% Fetal Calf Serum, 1 mM Sodium Pyruvate, 2 mM L-Glutamine, 1% Penicillin-Streptomycin, 0.1 mM MEM Eagle Non-essential Amino Acids, 1.5 g/L Sodium Bicarbonate, and 0.01 mg/ml insulin. Mel A2− and Mel A2+ were grown in DMEM medium supplemented with 10% Fetal Calf Serum, 1 mM Sodium Pyruvate, 2 mM L-Glutamine, 1% Penicillin-Streptomycin, 0.1 mM MEM Eagle Non-essential Amino Acids, 1.5 g/L.

Electroporation (EP): For each electroporation 1-8×10\hat{ }6 cells were harvested, and medium was replaced by washing three times in Optimem (Thermofisher). Cells were placed in Eppendorf tubes at a concentration of 10×10\hat{ }6 cells per ml. 3-10 ug of insert (DNA plasmid, mRNA, SiRNA or CAS9+sgRNA complex (RNP) were added per 10≢cells. For mock control the cells were electroporated without insert. The electroporation was done using either Amaxa Nucleofector (Lonza) or Nepa21 gene electroporator (Nepa Gene). Before electroporation, the cells were resuspended in either specific electroporation buffer for the Amaxa (Lonza) or in Optimem (Thermofisher) for theNepa21. Cells and insert were placed into a 2 mm electroporation cuvette for Nepa21 and cell project cuvette (Lonza) for Amaxa. Electroporation with the Nepa21 (NepaGene Unit) was performed using 225-250V pulse length of 2.5 ms. Electroporation with Amaxa was performed using cell project specific conditions defined by the device.

After run completion, 400 ul of pre-warmed RPMI medium was added to the cuvette and the cells were left in the cuvette for a 20-30 min recovery. The cells were transferred from the cuvette to 24 or 6-well plates at 10≢cells per ml.

Cells were maintained in culture from 2 hours to 3 days before being used for experiments.

Transfection of cells was conducted according to manufacturer's instructions. Briefly, 300K cells were plated in 6-well plates the day prior to the day of transfection. The following day, lipid nanoparticle mixes were prepared with 2 ug of DNA together with the Lipofectamin 3000 reagent (ThermoFischer cat #L3000015) and applied on cells in a gentle dropwise manner.

Transfer assay: 0.5-1×10\hat{ }5 K562, P815 or Mel A2+/−target cells were either labeled with Tag-it at final concentrations of 1.6 uM according to manufacturer's instruction (Biolegend cat #425101) or transfected with GFP or gene targeting sgRNA or PNA reporter system and seeded to 96 well U shape plates. 1-6×10\hat{ }5 T, YTS, or K562 effector cells were loaded with Cas9-sgRNA RNP complexes or were made to express Granzyme-Cherry, Granzyme-Cas9, Granzyme-meganuclease or Granzyme-meganuclease-GFP fusion constructs and were co-cultured with the target cells. Cells were co-incubated for 2-16 hours at 37° C. Transfer of fusion protein or RNPs was monitored by flow cytometry, fluorescence microscopy, or subsequent genomic sequence analysis in gene of interest for specific gene editing events that occurred as a result of introducing the gene editing proteins to the target cells. For re-directed transfer assay, P815 target cells were pre-coated for 1 hour, in PBS with 2 ug/ml of OKT-3 anti-CD3 Ab (Biolegend cat #BLG-317326) at room temperature, washed in PBS, and then co-cultured with target cells. Prior to co-culture with effector cells, target cells were treated with a pan-Caspase inhibitor Z-VAD-FMK (R&D Systems cat #FMK001) for 2 hours at 50 uM and its concentration was maintained following the addition of the effector cells and following the cell sorting procedure.

Cell staining for FACS analysis: Following co-culture, cells were stained for cells viability using the Fixable Viability Dye eFluor™ blue 450 (ThermoFisher cat #65-0863-18) or near-IR 633/635 (ThermoFisher cat #L10119). Blue and near-IR viability dye, Fixable Viability Dye eFluor™ 450 and 633/635 staining was carried out at room temperature in 100 microliters in U shape 96 well plates. Sample acquisition was done in the following devices: 5-laser Fortesa cell analyzer (BioRad), MacsQuant (Milteny), or BD FACSAria III cell sorter. In addition to measuring the viability dye, cells were also monitored for GFP, PNA RFP and mCherry signal. Prior to fluorescence analysis, forward side scatter gates were initially positioned in accordance with each cell type specific scatter patterns as determined for each cell type by itself. For mCherry signal analysis, target cells were then selected based on GFP expression or Tag-it labeling. For analyzing cells expressing the PNA reporter system, GFP expression was determined on cells gated for RFP expression. Intracellular staining using anti-Crispr/Cas9 Alexa Fluor 488 (Abcam cat #ab191468) and anti-Granzyme B Alexa Fluor 647 (Biolegend cat #515406) was performed with the intracellular Cytofix (Biolegend cat #420801) and Perm Wash (Biolegend cat #421002) buffers according to the manufacturer's instructions. Analysis was performed using the Flowjo (Becton Dickenson) or FCS Express (DeNovo) software. For cell sorting, the target cell population was isolated based on a two-log difference in Tag-it or RFP signal. Subsequently, target cells sorted after co-cultured with T or YTS cells were supplemented with Pan-caspase inhibitor Z-VAD-FMK (R&D Systems cat #FMK001) in a final concentration of 50 uM and an increased concentration of 2% penicillin-streptomycin. Fluorescence microscopy: Images of GFP, RFP, or mCherry expressing cells were captured with the Observer Z1 (Zeiss) fluorescent microscope and Axiovision software (Zeiss) using a range of 2-200 msec exposure.

Granzyme Knock-down: PBMCs were isolated as before. Activated T cells were transduced with retrovirus carrying an engineered antigen-specific TCR against melanoma cells on an HLA-2 background. After 2 days the transduced T cells were collected and plated in triplicate in 6-well plates.

The cells were then electroporated with GZMB-CAS9 fusion protein as well as an siRNA against the endogenous Granzyme B. The Granzyme B siRNA was designed to target the 3′ UTR of endogenously expressed Granzyme B. The siRNA does not recognize the Granzyme B-Cas9 fusion mRNA which contains only the open reading frame of Granzyme B. Two siRNAs were designed and synthesized, siGzmB-1 and siGzmB-2 (AxoLab, Germany). In order to evaluate the siRNA activity, T cells were electroporated with 300 pmol of either siGzmB-1 or siGzmB-2 and tested for Granzyme B protein levels using Granzyme B antibodies and FACS analysis. siGzmB-1 Sense strand: 5′g*g*AgCcAaGuCcAgAuUuA-3′ (SEQ ID NO: 45). Antisense strand 5′-U*A*aAuCuGgAcUuGgCuCc*u*u-3′ (SEQ ID NO: 46) siGzmB-2 Sense strand 5′-c*g*CuGuAaUgAaAcAcCuU-3′ (SEQ ID NO: 47) Antisense strand 5′-A*A*gGuGuUuCaUuAcAgCg*u*u-3′ (SEQ ID NO: 48) Lower case nucleotides denote 2′-O-methyl residues. Upper case nucleotides denote 2′-Fluoro residues. An “*” denotes a phosphorothioate backbone modification. Cells were either mock prepared with an empty vector, electroporated with only hGZMB_crmCherry or double electroporated with the expression plasmid and the siRNA.

The next day target cells were prepared. MART1 expressing melanoma cell lines either expressing HLA-A2 or not were used. Both cell lines were stained with CFSE to distinguish them from the effector T cells. Each well of lymphocyte cells was split in two to produce replicate plates and 2.5×10\hat{ }4 target cells were added to each well. The cells were allowed to co-culture for 3 hours and then were analyzed by flow cytometry to determine crmCherry transfer.

RNA-protein complex generation and nucleofection: Cas9 protein (Alt-R S.p. Cas9 Nuclease V3, IDT) or Cas9-GFP (CAS9GFPPRO, Sigma) were mixed with synthetic modified sgRNA. EMX1_1, Rosa26_1, GzmB_1 and GzmB_2, were ordered from Synthego, with 2′-O-methyl and 3′ phosphorothioate inter-nucleotide linkages at the first three 5′ and 3′ terminal RNA residues. For sgRNA sequences and target genes see Table 2. HBB1 sgRNA was ordered from AxoLab with multiple modification along the entire oligo c*u*u*GCC*C*Cf*Af *Cf *AfGGfGfCAGfUfAAgUUUUAGagcuagaaauagcaaGUUaAaA uAaggcuaGUccGUUAucAAc*u*u*g*a*a*a*a*a*gugG*ca*c*c*g*a*g*u*cg*g*u*g*c*u *u*u*u*u Capital letters indicate unmodified RNA. Lower case letters indicate 2′-O-methyl residues. “f” indicates 2′-Fluoro residues. “*” indicates phosphorothioate backbone modification. For RNA-protein preparation, 400-573 pmol of Cas9 or Cas9-GFP were mixed with 480-687 pmol sgRNA (keeping ratio of 1.2-1.3 of sgRNA to Cas9 protein) and incubated for 10-20 min in RT. The RNA-Protein complex and 400-571 pmol Electroporation Enhancer (IDT) was added to the cells just before electroporation.

TABLE 2
sgRNA sequences

| | | SEQ | |
|---|---|---|---|
| sgRNA | | ID | |
| name | Spacer sequence | NO: | Target gene |
| EMX1_1 | GUCACCUCCAAUGACUAGGG | 49 | EMX1 |
| Rosa26_1 | ACUCCAGUCUUUCUAGAAGA | 50 | Rosa26 |
| GzmB_1 | CUGACAGCUGCUCACUGUUG | 51 | Granzyme B |
| GzmB_2 | AAGUCUCUGAAGAGGUGCGG | 52 | Granzyme B |
| Luc_1 | UUGCACGAGAUCGCCAGCGGCGG | 75 | Luciferase |

RNA-Protein complex transfer assay: 10\hat{ }5 Tag-it labeled targets cells or Ab coated target cells (for activation) were seeded to 96 well U shape plates. After a 2-hour incubation, the target cells were washed and 6×10\hat{ }5 effector cells (T, K562 or YTS cells) post nucleofection were added to the target cells. 10 μl Caspase-8 inhibitor was again added for a final concentration of 50 μM. The cells were co-cultured at 37° C. for 4 hours.

Preparation of samples for SDS Page electroporation: Cells were lysed in RIPA buffer (×20 of cell pellet volume). RIPA buffer was 50 mM Tris-HCl, pH 8.0, with 150 mM sodium chloride, 1.0% Igepal CA-630 (NP-40), 0.5% sodium deoxycholate, and 0.1% SDS. Cells were lysed for 30 minutes at 4° C., followed by centrifugation for 10 minutes at 14000 RPM. The supernatant was transferred to new Eppendorf tubes and protein concentration was measured by Bradford reagent. To interpolate protein concentration, OD values were plotted to a reference graph of BSA amount vs OD.

Samples were mixed 1:4 in 4× Laemmli Sample Buffer (BioRad, cat #1610747) and then heated at 70° C. for 10 minutes. Samples were fun on Criterion™ TGX Stain-Free Precast Midi Gels. Running conditions were 50 minutes, 200V in Tris/Glycine/SDS running buffer (Biorad cat #161-0772). Gels were transferred using Trans-Blot TURBO (BioRad Cat #1704159) at 2.5 A and 25V for 10 min.

Immunoblotting: Blocking was done with 3% BSA in TBST (Cell Signaling Cat #99975) (0.1% Tween20) for 1 hour at room temperature (RT). The primary antibody used was Rabbit anti-Cas9 (Clontech Cat #632607). It was diluted 1:2500 in blocking solution and incubation was for 1.5 hours at RT. The secondary antibody was Goat anti Rabbit HRP (Jackson Cat #115-035-062). It was diluted at 1:10000 in blocking solution and incubation was for 1 hour at RT. Blots were stained with ponceau-red to confirm loading uniformity.

### Example 1: Primary T Cells and the NK Cell Line YTS Efficiently Transfer Granzyme-Cherry Protein to Target Cells

In order to test the ability of a Granzyme B fusion protein to transfer a protein of interest to a target cell, a Granzyme B-mCherry fusion protein was created. mCherry was used (SEQ ID NO: 1). The first 11 amino acids, MVSKGEEDNMA (SEQ ID NO: 4), were removed due to possible cleavage sites. This produced the still fluorescent truncated protein crmCherry (SEQ ID NO: 3). Inactive human Granzyme B (G19A/E20A) was placed N-terminal to the crmCherry with a glycine-serine linker (SEQ ID NO: 11) between them. The nucleic acid sequence encoding this fusion protein was inserted into the pMAX expression vector to produce a pMAX-hGZMB-crmCherry vector (SEQ ID NO: 29) (FIG. 1).

In order to assess the subcellular localization profile of the Granzme B_crmCherry fusion protein, the pMAX-hGZMB-crmCherry vector was expressed in primary human CD8 positive T cells isolated from healthy donors. Cherry fluorescence was confirmed in the electroporated cells.

Human K562 chronic myelogenous leukemia cells (human erythroleukemic cell line) were used as the target cell. The cells were electroporated with pMaxGFP vector and GFP fluorescence was confirmed in the target cells. The primary T cells were also confirmed to be GFP negative and the K562 cells were confirmed to be Cherry negative.

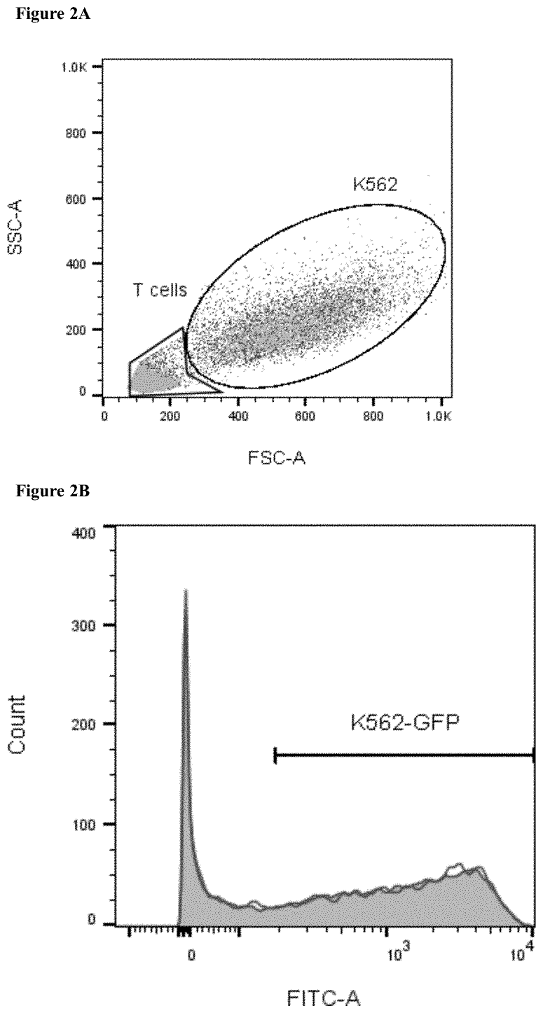

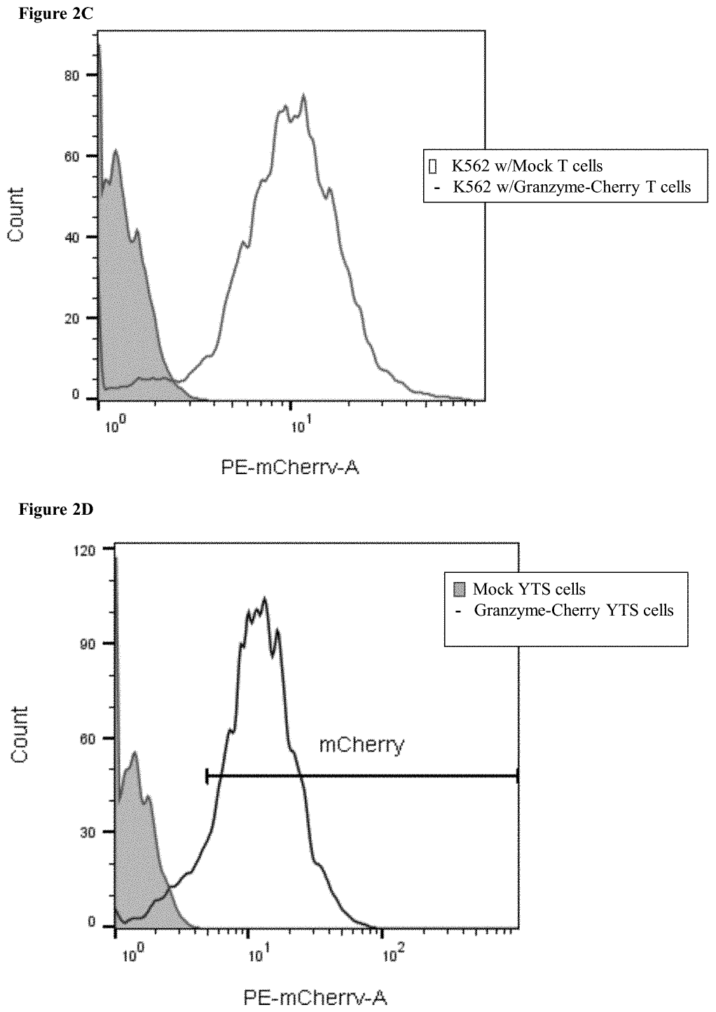

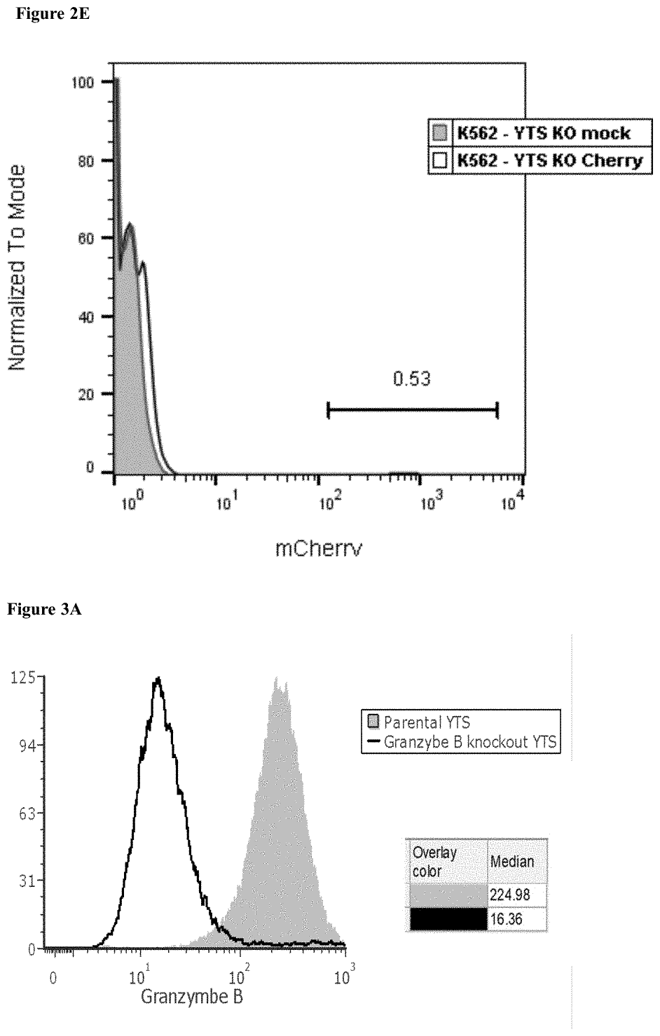

Electroporated primary T cells were co-cultured with electroporated K562 cells at an effector to target ratio of 6:1 for 4 hours. Following the co-culture, the cells were analyzed by flow cytometry. The K562 cells could be separated by gating on the target cell population using only forward- and side-scatter (FIG. 2A). In order to further ensure that only K562 cells are being analyzed, GFP positive cells were selected within this population (FIG. 2B). When this GFP positive population was analyzed for Cherry expression, it was found to be highly Cherry positive (FIG. 2C). Indeed, greater than 90% of the GFP positive K562 cells (94.8%) were found to be also Cherry positive. When control cells electroporated with an empty vector were used for the co-culture, the K562 cells were 100% Cherry negative as would be expected (FIG. 2C).

These experiments were repeated with cells of the YTS NK cell line in place of the primary T cells. After co-culture, the K562 cells were again gated based first on forward- and side-scatter and then on GFP expression. The target cancer cells were once again found to be highly Cherry positive (FIG. 2D) with 88.1% of the cells found to express Cherry. These results, taken together, show the high efficiency by which T cells and NK cells can transfer Granzyme fusion proteins to target cells. Importantly, when a mRNA IVT product of the coding region of a crmCherry expressing plasmid alone (without fusion to Granzyme) was expressed in GZMB-KO YTS cells, no transfer of Cherry to Tag-it positive K562 target cells was observed (FIG. 2E).

### Example 2: Granzyme B Knock-Down and/or Knockout Potentiates Transfer of Granzyme B-crmCherry to Target Cells

In order to test the effect of endogenous Granzyme knock-down on the transfer of exogenous Granzyme B fusion proteins, an siRNA molecule against only endogenous Granzyme B was utilized (see Materials and Methods). Primary human T cells were isolated and transduced with a retroviral vector bearing an antigen-specific engineered TCR which is reactive to MART1 and restricted to HLA-A2. These cells were also electroporated with a mRNA IVT product encoding hGZMB_crmCherry (plasmid 1_3 in FIG. 1) with or without an siRNA knock-down of endogenous Granzyme B.

Target melanoma cells employed in this assay express HLA-A2 and the surface melanoma antigen MART1, or only MART1 but not HLA-A2. These target cells were stained with CFSE which allows them to be easily distinguished from the T cells. The T cells were co-cultured with the target melanoma cells for 4 hours and then Cherry expression in the CFSE positive cells was assessed. In the HLA-A2 expressing melanoma cells, the T cells with knockdown of endogenous Granzyme B produced a 25% increase in mCherry fluorescent cells as compared to co-culture with non-knockdown cells.

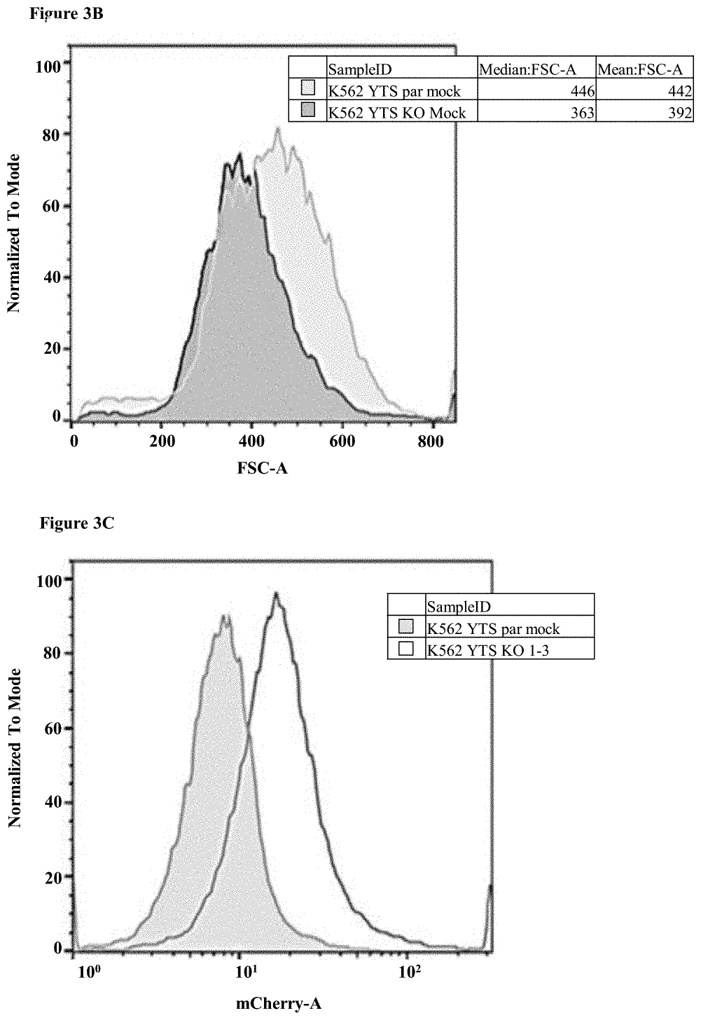

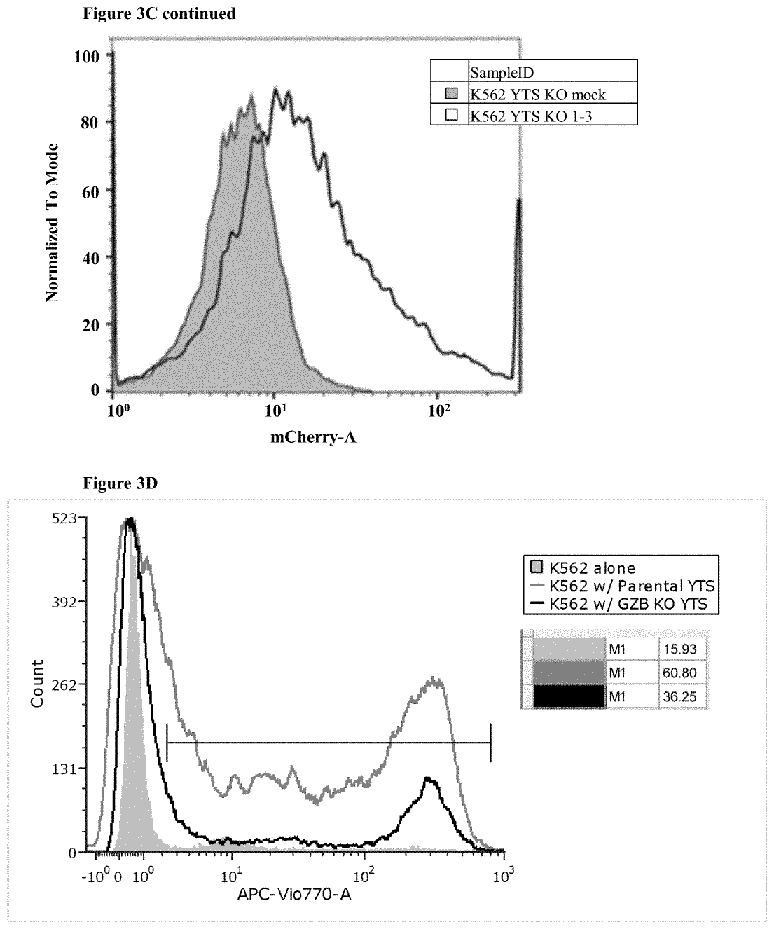

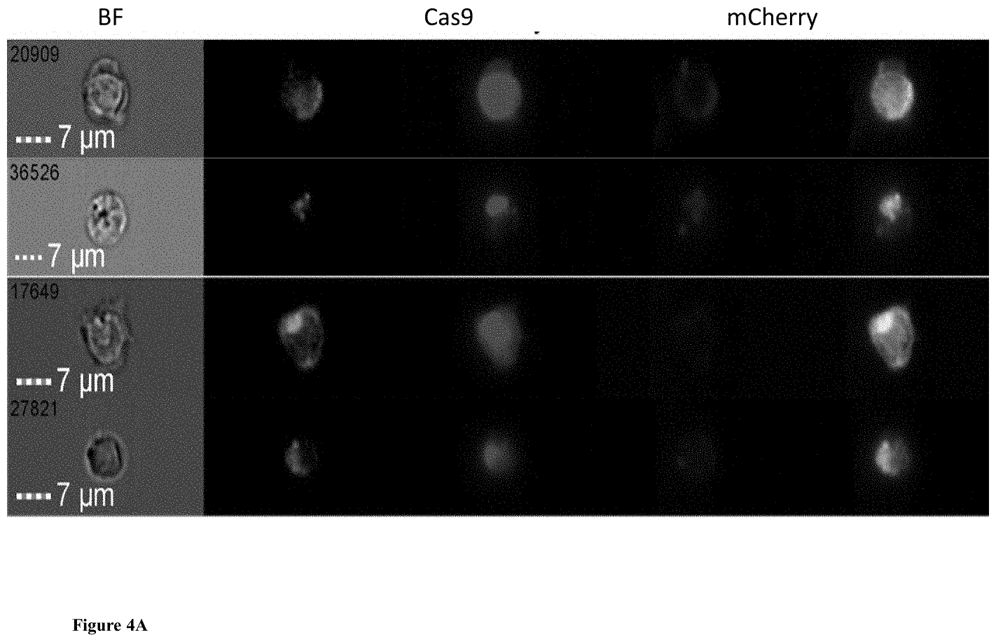

Further, cell lines were generated in which the endogenous Granzyme B is knocked out. YTS cells were electroporated by Nepa gene electroporator (200 volt, 5 ms), with RNA-Protein complex for CRISPR knockout. Alt-R® S.p. Cas9 Nuclease V3 (IDT) was incubated with either GzmB_1 (SEQ ID NO:51) or GzmB_2 (SEQ ID NO:52) synthetic single guide RNA (sgRNA, Synthego) against Granzyme B to produce stable RNA-protein complex. The YTS cells were electroporated with either RNA-protein complex containing only sgRNA GzmB_1, only GzmB_2 or with a mixture of both complexes in a 1:1 ratio. These complexes were electroporated into the YTS cells and the cells were allowed to recover in culture for 3 days. Granzyme B expression in the YTS cells was measured after 10 days in culture. As shown in FIG. 3A, YTS cells electroporated with RNA-Protein GzmB_1 showed nearly 95% reduction in Granzyme B expression relative to parental YTS cells. YTS cells electroporated with RNA-Protein GzmB_2 or mixture of GzmB_1 and GzmB_2 showed significantly lower percentage of Granzyme B reduction (data not shown).

In order to confirm that the knockout cells indeed have reduced cytotoxic effect, GZMB-KO YTS cells were co-cultured with K562 target cells for either 24 or 48 hours. At 24 hours of co-culture, the percentage of K562 cells gated within the forward scatter typical for untreated cells was significantly higher in K562 cells co-culture with the GZMB KO cells as compared to those co-cultured with non-KO YTS cells. This can be seen by the higher average FCS in the cells co-cultured with the non-knockout cells (FIG. 3B). The significant increase in size in K562 co-cultured with parental YTS expressing Granzyme B is typical of osmotic instability induced as a result of perforin-granzyme cell death pathway and is absent in the K562 co-cultured with GZMB KO YTS cells which display a similar size and granularity as naïve non-treated K562 cells.

To further validate the reduced cytotoxic effect of the GZMB-KO YTS cells, viability of K562 cells was tested after 48 hours of co-culture with parental or KO YTS NK cells. Cells were stained with viability dye (ThermoFisher) as described herein above. As seen in FIG. 3D, K562 cells cultured with GZMB-KO YTS cells exhibit reduced dead cell staining (36%) relative to K562 cultured with parental YTS cells (60%). Thus, the GZMB-KO YTS cell line exhibited reduced cytotoxic/lytic activity in comparison to the parental YTS cell line.

This same co-culture system was used to check mCherry transfer. The GZMB-KO cells and parental YTS cells were transfected with the plasmid expressing granzyme fused to crmCherry (plasmid 1_3 in FIG. 1). They were then co-cultured with K562 cells for 4 hours as was done previously (Example 1) and the mCherry expression was measured in the target cells. As can be seen in FIG. 3C, not only did the KO cells effectively transfer mCherry, but the transfer efficiency it actually increased (4.6×MFI ratio increase over negative cells for KO vs. 2.8× for WT Granzyme), again attesting to the superiority of using non-cytotoxic cells, and particularly GZMB-KO cells, for transfer.

Taken together these results point-out for effector cells with reduced cytotoxicity as potent delivery vehicles.

### Example 3: Granzyme B Fused to Cas9 Transfers to Target Cells

Having established that inactive Granzyme B can successfully transfer mCherry to target cells it was next tested if significantly larger proteins could be transferred in this manner and retain enzymatic activity within the target cells. mCherry has a molecular weight of only about 27 kDa, and the crmCherry is even smaller with a weight of only about 25.5 kDa. In contrast, Cas9, a genome-editing protein, has a molecular weight of over 163 kDa. A second genome-editing protein, meganuclease which is specific to PCSK9, was also investigated. The meganuclease has a molecular weight of about 37 kDa.

The full Cas9 coding sequence was placed after Granzyme B with a linker (GS linker) between them in the pMax expression plasmid (see FIG. 1). The Cas9 also contained an N-terminal HA tag. In order to easily track cells that received the plasmid, crmCherry was inserted downstream of Cas9. A P2A self-cleaving peptide was placed between the Cas9 and the crmCherry. This should result in Cherry expression in the cells that are effectively electroporated, but since the Cherry would be uncoupled from the Granzyme it would not be expected to reach granulocytic vesicles or be transferred. Indeed, when the pMAX-hGZMB_HA-Cas9_P2A_crmCherry vector was expressed in HEK293 cells, the Cherry had a diffuse cytoplasmic staining similar to what was observed when crmCherry was expressed alone. In contrast the Cas9 showed punctate staining indicating entrance into vesicles similar to that observed for Granzyme_crmCherry (data not shown). Meganuclease was also linked to Granzyme B, but with an OLLAS flexible linker. As meganuclease is much smaller than Cas9, GFP could be directly conjugated to the meganuclease itself without the need for a P2A peptide (see FIG. 1).

In order to confirm that the new fusion proteins retained their DNA editing activity, a reporter system (PNA Bio) was employed. Granzyme B is a sufficiently large protein that there is a concern, especially when the linker is not cleavable, that it could impair the function of the editing protein. The PNA reporter system carries genes encoding two fluorescent proteins (RFP and GFP) linked by a specific editing targeting sequence. The RFP and GFP coding sequences are designed to express the red but not the green fluorescent protein as it is placed out of frame. The system contains two GFP encoding sequences, one placed −1 frameshift from the RFP and one in −2 frameshift from the RFP. Upon expression of an editing nuclease, a double strand break is induced at the editing target site between the RFP and GFP expressing sequences, leading to frameshift mutations and expression of GFP. Following GFP expression, normalized by RFP in cells, demonstrates if the editing nuclease is functionally cutting and to what extent. For evaluating the editing efficiencies of the fused Cas9, K562 cells were electroporated by Nepa gene electroporator (250 volts 2.5 seconds) with 5-8 pg of EMX1_1-PNA reporter system plasmids (PNA Bio), and 2 ug of a plasmid expressing EMX1_1 guide (SEQ ID NO: 49). A GZMB-CAS9 fusion protein which lacked the ER signal peptide (ERSP) was expressed (2 ug) and as a positive control, Cas9 expressing plasmid was also expressed (2 pg of Cas9 fused to blue fluorescent protein). Removal of the ERSP was necessary to ensure cytoplasmic expression and nuclear localization of the fusion protein within the K562 cells. The same experiment was performed using expression of the meganuclease fusion protein also lacking the ERSP (2 ug) and a PCSK9-PNA reporter system plasmid (5 ug). Meganuclease expressing plasmid was used as a positive control (2 ug). Following 3 days in culture, the editing efficiencies were evaluated by following the GFP signal normalized to the RFP signal by FACS analysis. The results are presented in Table 3. The fusion proteins containing CAS9 or meganuclease both retained their functionality.

TABLE 3
Editing quantification with PNA editing system

| | | Percent GFP |
|---|---|---|
| | | positive within |
| Plasmid | Description | RFP positive |
| PNA only | Negative control | 3.65% |
| PCSK9-PNA and meganuclease | Positive control | 73.34% |
| PCSK9-PNA and SP free GZMB- | Fusion with | 26.25% |
| meganuclease (construct 2_6 | flexible linker | |
| in FIG. 1) | | |
| EMX-PNA, gRNA and CAS9-BFP | Positive control | 93.90% |
| EMX-PNA, gRNA and SP free | Fusion with | 21.62% |
| GZMB-CAS9 (construct 1_4 in | flexible linker | |
| FIG. 1) | | |
| EMX-PNA, gRNA and SP free | Fusion with | 30.22% |
| GZMB-CAS9 (construct 1_6 in | cleavable linker | |
| FIG. 1) | | |

Next, the ability of the fusion proteins to edit the endogenous genome was tested. In the PNA system, many copies of the target plasmid may be present in a given cell, making editing more likely. However, in an endogenous diploid genome there are only two copies of the target sequence. To test the ability of the fusion proteins to cut endogenous targets, a similar experiment was performed only without the PNA plasmid. The control plasmids and the test plasmids were expressed in K562 cells. The sgRNA was expressed along with the CAS9 plasmids. Following 3 days in culture, genomic DNA was extracted using QuickExtract™ DNA Extraction Solution (Lucigen). The region flanking the editing target was amplified by PCR (Q5 DNA Polymerase, NEB) and the products were sent for next-generation sequencing. Primers used are provided in Table 5. The number of editing events was calculated over the total number of reads and editing efficiencies were normalized to fluorescent cells (indicating insert positive events) as determined by FACS analysis. The results of the genome-editing are summarized in Table 4. The fusion proteins containing CAS9 or meganuclease both retained their genomic editing functionality.

TABLE 4
Editing quantification of genomic loci

| | | Editing in |
|---|---|---|
| Plasmid | Description | genome, % |
| PCSK9 Meganuclease (SEQ ID | Positive control | 76% |
| NO: 5) | | |
| ERSP free GZMB Meganuclease | Fusion with flexible | 15% |
| (construct 2_5 in FIG. 1; SEQ | linker | |
| ID NO: 70) | | |
| CAS9-BFP | Positive control | 97% |
| GZMB-CAS9 lacking ERSP | Fusion with flexible | 30% |
| (construct 1_16 in FIG. 1; SEQ | linker | |
| ID NO: 66) | | |
| GZMB-CAS9 lacking ERSP | Fusion with cleavable | 19.5% |
| (construct 1_17 in FIG. 1; SEQ | linker | |
| ID NO: 67) | | |
| GZMB-CAS9 lacking ERSP | Fusion with cleavable | 14% |
| (construct 1_18 in FIG. 1; SEQ | linker | |
| ID NO: 68) | | |

TABLE 5
Primer list

| | | | SEQ |
|---|---|---|---|
| | | | ID |
| Target | Name | Sequence | NO: |
| hEMX1 | oETX54_hEMX1gPCRf | ACACTGACGACATGGTTCTACAGTTC | 55 |
| | | CAGAACCGGAGGACAAAGTAC | |
| | oETX55_hEMX1gPCRr | TACGGTAGCAGAGACTTGGTCTTCCA | 56 |
| | | GGCCTCCCCAAAGC | |
| mRosa | oETX46_mROSAgPCRf | ACACTGACGACATGGTTCTACAGAG | 57 |
| | | GCGGATCACAAGCAATA | |
| | oETX47_mROSAgPCRr | TACGGTAGCAGAGACTTGGTCTGGG | 58 |
| | | AGGGGAGTGTTGCAA | |
| hPCSK9 | oETX48_hPCSK9gPCRf | ACACTGACGACATGGTTCTACACCTG | 59 |
| | | GCTATGGTAGGGACAG | |
| | oETX49_hPCSK9gPCRr | TACGGTAGCAGAGACTTGGTCTGCAG | 60 |
| | | TGGGGTGGTGACTTAC | |
| hHbb | oETX50_hHBBgPCRf | ACACTGACGACATGGTTCTACACCAA | 61 |
| | | CTCCTAAGCCAGTGC | |
| | oETX51_hHBBgPCRr | TACGGTAGCAGAGACTTGGTCTAGTG | 62 |
| | | CCTATCAGAAACCCAAG | |
| PNA | oETX67_PNAngsF | ACACTGACGACATGGTTCTACACCAC | 63 |
| | | AACGAGGACTACACCA | |
| | oETX68_PNAngsR | TACGGTAGCAGAGACTTGGTCTTGGT | 64 |
| | | GCAGATGAACTTCAGG | |

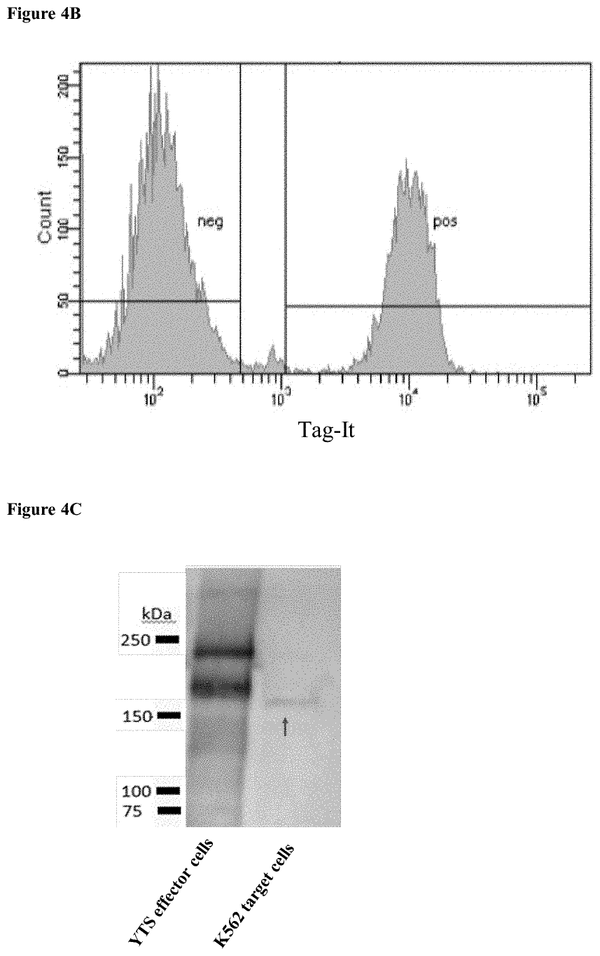

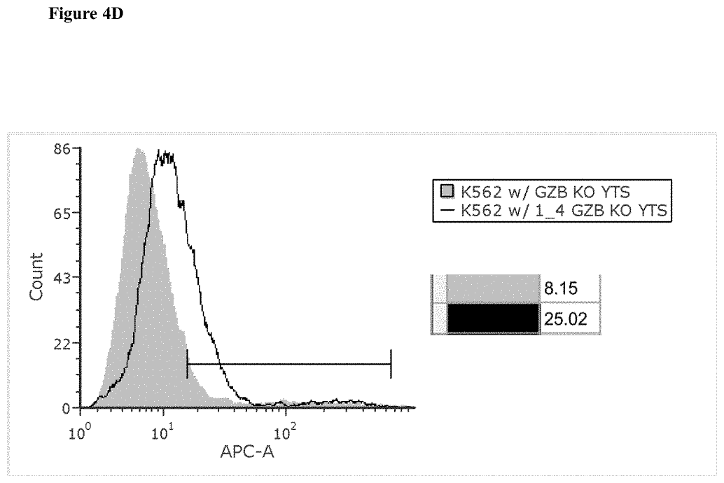

As it is now confirmed that the fusion proteins are still functionally able to induce editing, next, the expression profile of the fusion proteins in Granzyme B and perforin expressing cells was evaluated. pMAX-hGZMB_HA-Cas9_P2A_crmCherry vector was electroporated into primary CD8 T cells. 48 hours after electroporation the cells were stained with an anti-Granzyme B antibody as well as an anti-Cas9 and were imaged along with the crmCherry. As can be seen in FIG. 4A, which shows 4 representative cells, Cas9 and Granzyme B colocalized within vesicles while mCherry showed a diffuse cytosolic pattern. This indicates that even with its large size Cas9 can enter lytic granules within lymphocytes.

As it is now confirmed that the fusion proteins are expressed in effector cells expressing Granzyme B and perforin in an expression profile characteristic of lytic granules, the transfer of CAS9 and meganuclease fused to Granzyme B from effector to target cells was evaluated next. 1×10\hat{ }6 YTS cells were electroporated with 10 ug of mRNA IVT product encoding Granzyme B-CAS9 using the Nepa gene unit at 225V with a pulse length of 2.5 ms. Fusion protein expressing cells were co-cultured with target cells (K562) in a 1:6 effector to target cell ratio for 4 hours and the transfer of Cas9 was monitored. The target cells were Tag-it labeled and therefore could be easily separated from the NK cells by their Tag-it fluorescence (FIG. 4B). Following separation of the Tag-it positive cells using a FACS Aria III FACS sorter, the sorted cells were lysed and run on a western blot. Expression of Cas9 protein in these cells was determined by blot hybridization with an anti-Cas9 antibody. As can be seen in FIG. 4C, CAS9 protein was detectable in the K562 cells indicating that even such a large protein could be effectively transferred by the lytic granule. Notably, in the lymphocytes themselves two bands are detected. These are the Granzyme-CAS9 fusion protein before cleavage (expected size 190 kDa) and the already cleaved CAS9 (expected size 160 kDa). As cleavage is ongoing in the lytic vesicle, both proteins are detected. In the target cell, only the lower band is detected as by this point the CAS9 is fully separated from the Granzyme.

Next, YTS Knockout effector cells (generated as described above) were used to evaluate transfer of Granzyme-CAS9 fusion protein to K562 target cells. Since no endogenous Granzyme B is produced in the knockout effector cells, transfer of the fusion protein to target cells was evaluated by FACS detection of Granzyme B, using intracellular staining with Alexa Fluor 647 labeled anti-Granzyme B antibodies, in Tag-it positive K562 target cells. As seen in FIG. 4D, Granzyme B was clearly present in K562 cells cultured with GZMB-CAS9-expressing GZMB-KO YTS but was absent from K562 cells cultured with mock transfected GZMB-KO YTS cells. This clearly demonstrates that GZMB-KO YTS effector cells can transfer GZMB-CAS9 fusion protein.

### Example 4: Gene-Editing after Granzyme Mediated Transfer

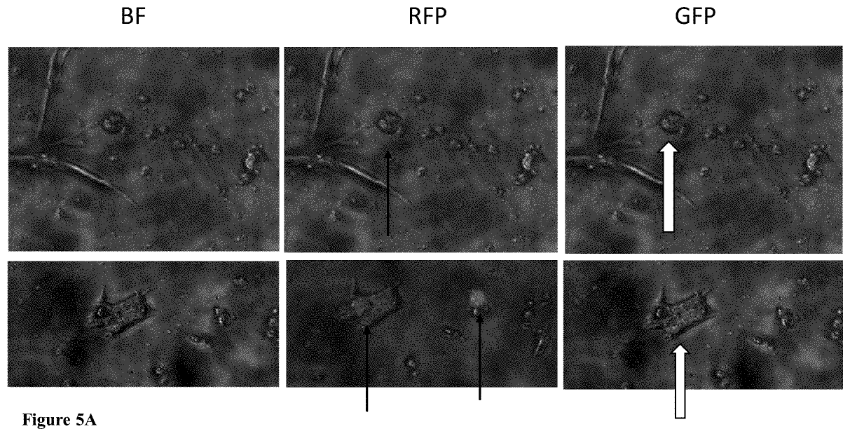

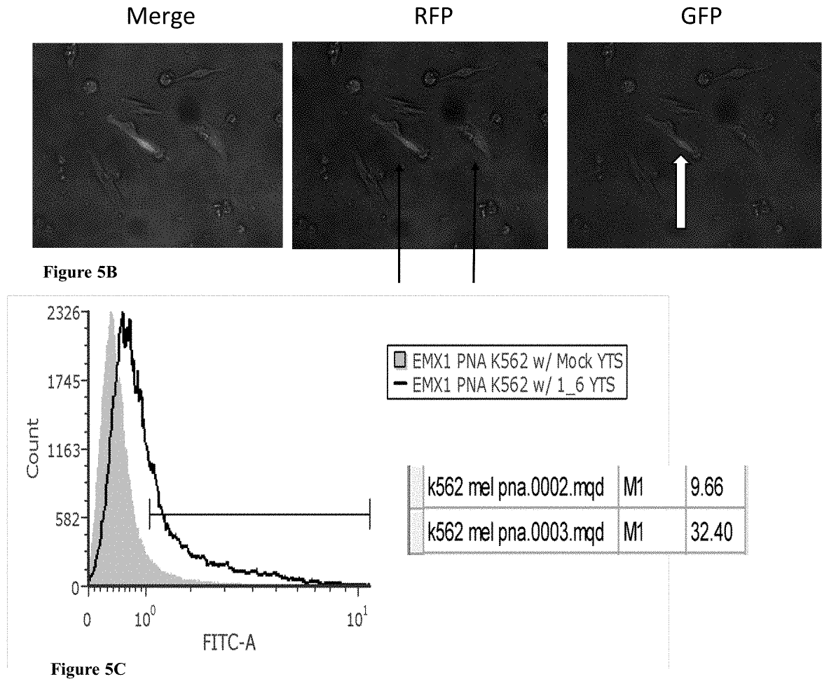

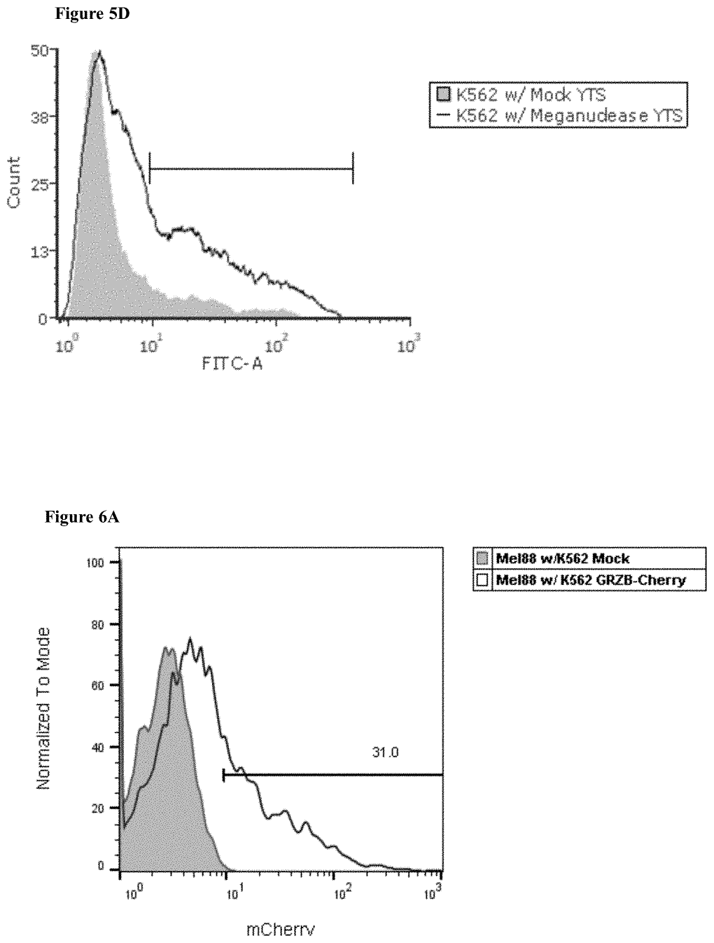

The ability of CAS9 and meganuclease transferred by Granzyme fusion to carry out genome-editing in the target cells was assessed. CD8 cells were isolated and activated as before. Electroporation of Granzyme-Cas9 mRNA into the CD8 cells and into YTS cells was carried out as before. K562 and melanoma target cells were co-cultured as before and electroporated with the EMX_1 sgRNA encoding plasmid and the PNA-reporter plasmid with an EMX sequence in the cleavage target area. The target cells were electroporated with a NepaGene unit at 250V with a pulse length of 2.5 ms. Cells were allowed to recover and 10\hat{ }5 cells were seeded in U shaped plates. To this, 6×10\hat{ }5 T cells or YTS cells with Granzyme-Cas9 or Granzyme-meganuclease were added. T cells or YTS cells electroporated without an insert were used as control. The cells were co-cultured for 4 hours. RFP positive target cells were separated from RFP negative effector cells by a FACSAria III FACS sorter, and GFP within the RFP positive population was monitored as evidence for gene-editing events. As can be seen in FIG. 5A-B, cleavage in the target cells occurred as evidenced by the expression of GFP in RFP-positive cells. Thus, it is clear that not only can the CAS9 be transferred from the effector to the target cell by fusion with Granzyme B, but the CAS9 is fully functional and capable of inducing editing in the target cells. Similar results were obtained using T cells (FIG. 5B) and YTS cells (FIG. 5A) and both in target K562 and melanoma cells.

The editing efficiency of Granzyme-Cas9 transferred by YTS cells was also evaluated using a GZMB-CAS9 fusion protein with a linker sequence (SEQ ID NO: 2) between the Granzyme and the CAS9 that is predicted to be cleaved in acidic pH (plasmid 16, SEQ ID NO: 33). mRNA IVT product of the coding region of this construct was electroporated into YTS cells as before. The editing activity of CAS9 within K562 target cells expressing the PNA-reporter plasmid with an EMX sequence in the cleavage target area and an EMX_1 sgRNA encoding plasmid was tested. Effector cells expressing GZMB-CAS9 were co-cultured with the above mentioned K562 target cells. After 4 hours of co-culture, target cells were sorted based the RFP signal as described herein above and grown for 3 days. The editing efficiencies were evaluated by following the GFP signal normalized to the RFP signal by FACS analysis. As shown in Figure FIG. 5C, a substantial three-fold shift in GFP fluorescence is observed in K562 target cells cultured with GZMB-CAS9 expressing YTS effector cells as compared to K562 target cells cultured with mock transfected effector YTS cells. This indicates that CAS9 transferred by YTS NK cells to K562 target cells retains its editing function even after separation from GZMB in the acidic pH of the lytic granules.

The editing efficiency of Granzyme-Meganuclease transferred by YTS cells was evaluated using a GZMB-Meganuclease fusion protein (plasmid 2_4; SEQ ID NO: 69). mRNA IVT product of the coding region of this construct was electroporated into YTS cells as before. The editing capability of the PCSK9-Meganuclease within K562 target cells, expressing the PNA-reporter plasmid with a PCSK9 sequence in the cleavage target area was tested. Co-culture between effector and target cells and sorting of RFP positive target cells was performed as described herein above. The editing efficiencies were evaluated after 3 days by following the GFP signal normalized to the RFP signal by FACS analysis. As shown in figure FIG. 5D, a substantial shift in GFP fluorescence is observed in K562 target cells cultured with GZMB-Meganuclease expressing YTS effector cells relative to K562 target cells cultured with mock transfected effector YTS cells. This indicates that the GZMB-Meganuclease fusion protein is being transferred by YTS NK cells to K562 target cells and retains its editing function.

The editing efficiency of Granzyme-Cas9 transferred by YTS cells was also evaluated using a GZMB-CAS9 fusion protein with a linker sequence (SEQ ID NO: 2) between the Granzyme and the CAS9 that is predicted to be cleaved in acidic pH (plasmid 16, SEQ ID NO: 33). mRNA IVT product of the coding region of this construct was electroporated into YTS cells as before. The editing activity of CAS9 within K562 target cells expressing a plasmid encoding the PNA-reporter plasmid with a Luciferase sequence in the cleavage target area and a Luciferase_sgRNA was tested (SEQ ID NO: 75). Effector cells expressing the above mentioned Granzyme B-CAS9 were co-cultured with the above mentioned K562 target cells. Cells were grown for 48 hours in co-culture after which editing efficiencies were evaluated by DNA extraction and PCR amplification of the flanking region of the recognition site of the Luciferase sgRNA. YTS mediated transfer of Granzyme B-CAS9 resulted in 15.7% of editing in K562 cells. This indicates that CAS9 transferred by YTS NK cells to K562 target cells retains its editing function even after separation from GZMB in the acidic pH of the lytic granules.

Having shown editing of the exogenous PNA plasmid, editing of the endogenous genome was now tested. Genome-editing mediated by transfer of the Granzyme B-CAS9 fusion protein in co-culture was performed as described hereinabove. Target cells were electroporated with a plasmid encoding EMX-1 sgRNA fused to mCherry. After 4 hours of co-culture, target cells were sorted based on mCherry signal and incubated for 3 days, after which genomic DNA was extracted. Edited reads were quantified by next generation sequencing. T cell transfer Granzyme B-CAS9 fusion protein produced an editing of 2% in melanoma cells, 1.25% in K562 cell and YTS cell transfer into K562 cells produced 0.71% editing, normalized to viable cells and transfer efficiency. This demonstrates that endogenous editing is possible in target cells that received the genome-editing protein by Granzyme mediated transfer.

The ability of PCSK9-specific meganuclease to carry out gene editing in the genome of the target cell, following Granzyme B mediated delivery, was assessed. Transfection of mRNA IVT product of the coding region of Granzyme-meganuclease from plasmid 2_4 into YTS cells was carried out as before. K562 target cells were labeled with Tag-it as described herein above. Effector and target cells were co-cultured for 4 hours after which target cells were sorted by a FACS Aria III FACS sorter based on Tag-it signal. Sorted target cells were cultured for 3 additional days and genomic DNA was extracted. Edited reads were quantified by next generation sequencing. YTS cell transfer of GZMB-meganuclease into K562 cells produced 1.4% genome editing after normalizing for the viable cells and transfer efficiency. This demonstrates that endogenous editing is possible in target cells that received the genome-editing protein by Granzyme mediated.

### Example 5: Myeloid Cells are Capable of Transferring Genome-Editing Proteins

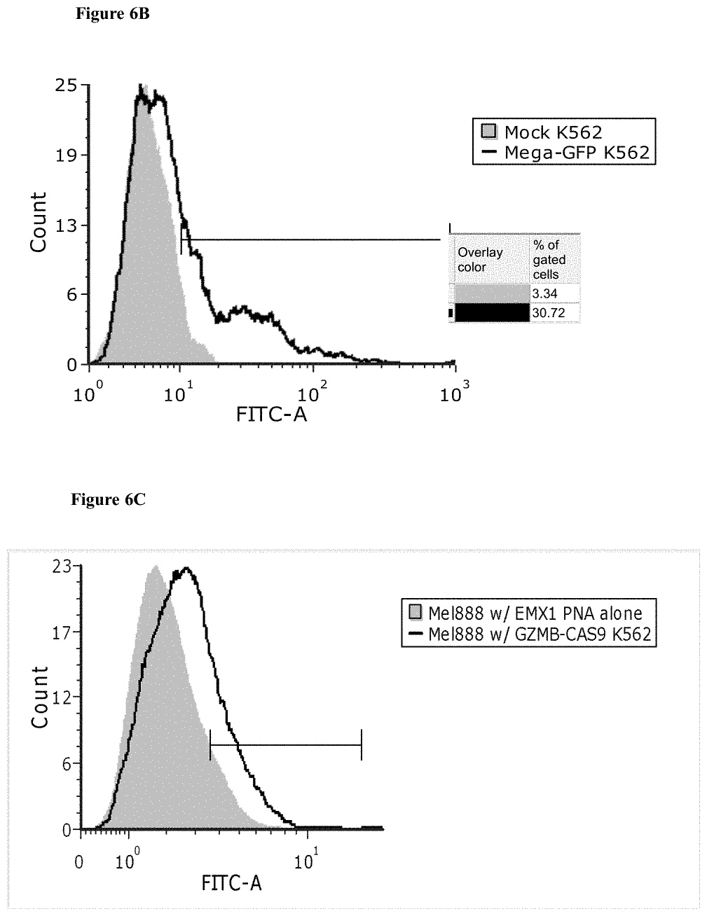

Myeloid cells are known to also express perforin and granzyme similar to lymphocytes, but they do not produce lytic granules. K562, Chronic Myeloid Leukemia cell line was used as an experimental model to test the ability of myeloid cells to successfully transfer cargo. To investigate whether K562 can be used as a potent delivery vehicle, first the transfer of crmCherry fused to Granzyme B from K562 cells to melanoma cells was tested. K562 cells were electroporated with mRNA IVT product of the coding region of plasmid 1_3 as described hereinabove. K562 cells and Tag-it labeled melanoma cells were co-cultured for 4 hours and mCherry signal within the Tag-it positive cells was monitored. As can be seen in FIG. 6A, ˜30% of the Tag-it positive target melanoma cells were detected as being mCherry positive. This clearly demonstrates that myeloid cells are also capable of Granzyme mediated protein transfer.

The transfer of meganuclease by K562 as effector cells was tested next. K562 cells were electroporated with 5 ug of plasmid 2_6 (shown in FIG. 1) encoding a human mutant Granzyme B linked to meganuclease and GFP. The K562 cells were incubated with target melanoma cells stained with Tag-it and co-cultured for 4 hours. GFP was detected in the Tag-it positive cells (˜30% of Tag-it positive cells) indicating that the meganuclease was successfully transferred (FIG. 6B). Thus, surprisingly, myeloid cells were also competent to transfer genome-editing proteins.

The ability of CAS9 and meganuclease transferred by Granzyme fusion to carry out editing in target cells was assessed. K562 cells were electroporated with Granzyme-PCSK9-specific meganuclease mRNA IVT products coding sequence of plasmid 2_4 as described herein above. Manipulated K562 cells were co-cultured with the melanoma target cells transfected with PNA-reporter plasmid with an PCSK9 sequence in the cleavage target area (for meganuclease) using lipofectamine 3000 as described hereinabove. 5×10\hat{ }4 transfected melanoma target cells were seeded in U shaped 96 well plates. To this, 2×10\hat{ }5 K562 cells with Granzyme-PCSK9 meganuclease were added. K562 cells electroporated without an insert were also used as a control. The cells were co-cultured for 4 hours. RFP positive target cells were separated from RFP negative effector cells by a FACSAria III FACS sorter. Sorted cells were kept in culture for 3 days after which GFP within the RFP positive population was monitored as evidence for gene-editing events. As can be seen in FIG. 6C, cleavage in the target cells occurred as evidenced by the expression of GFP in RFP positive melanoma target cells. Thus, it is clear that not only can the editing nuclease be transferred from myeloid effector cells to target cells by fusion with Granzyme B, but the nucleases are also fully functional and capable of inducing editing in the target cells.

The ability of PCSK9-specific meganuclease to carry out gene editing in the genome of melanoma target cells, following Granzyme B mediated delivery by K562 as effector cells, was assessed. Electroporation of mRNA IVT product of the coding region of Granzyme-meganuclease (plasmid 2_4 described in FIG. 1) into YTS cells was carried out as before. K562 target cells were labeled with Tag-it as described herein above. Effector and target cells were co-cultured for 4 hours after which target cells were sorted by a FACS Aria III FACS sorter based on Tag-it signal. Sorted target cells were cultured for 3 additional days and genomic DNA was extracted. Edited reads were quantified by next generation sequencing. K562 cell transfer of GZMB-meganuclease into melanoma cells produced 2% genome editing after normalizing for the viable cells and transfer efficiency. This demonstrates that endogenous editing is possible in target cells that received the genome-editing protein by Granzyme mediated.

### Example 6: T Cells and NK Cells Mediate Transfer of CAS9+gRNA Complex to Target Cell

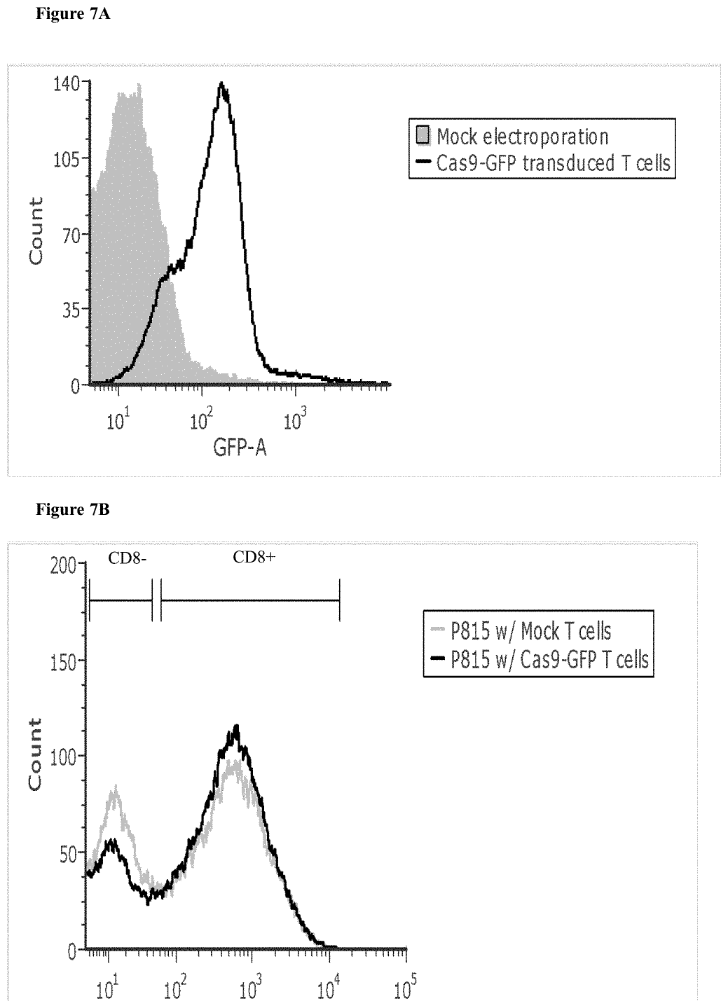

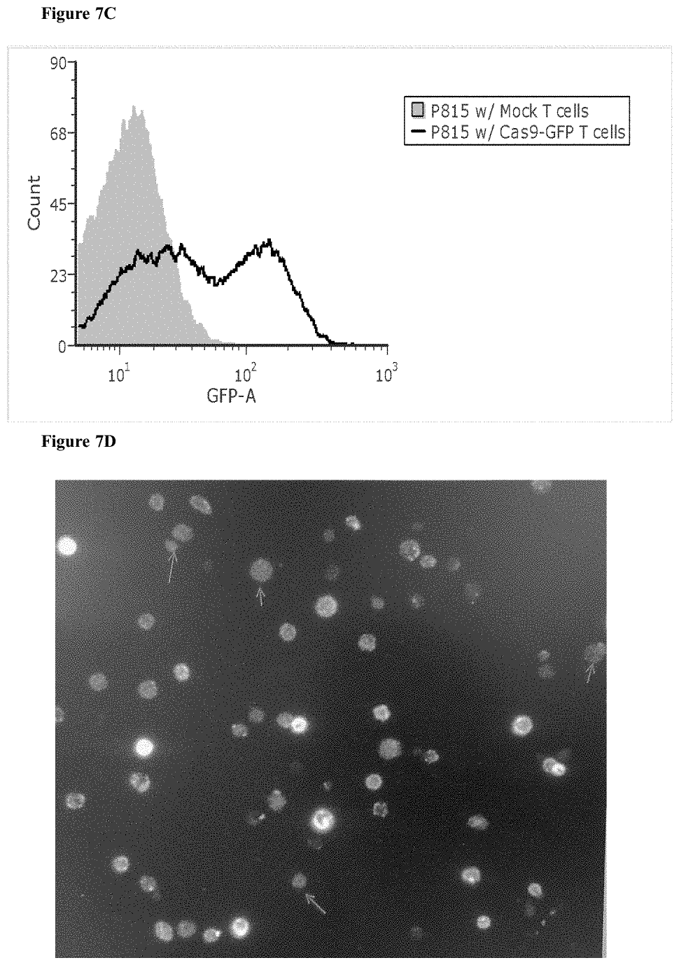

In addition to Granzyme B mediated transfer, direct transfer of protein-RNA complex by lymphocytes was also tested. Primary CD8 positive T cells were isolated and activated as before. Cas9-GFP was incubated with a synthetic single guide RNA (sgRNA) against EMX1 to produce stable RNA-protein complex. This complex was electroporated into the primary T cells with the Nepa21 (NepaGene Unit) at 225V and pulse length of 2.5 ms. Following a short recovery culture GFP expression in the T cells was confirmed (>90% GFP positive) (FIG. 7A).

Two different target cells were labeled with CFSE as before: human K562 cells and mouse p815. The electroporated T cells and labeled target cells were co-cultured for 4 hours as before. After co-culture the cells were stained with a viability dye (Near infrared APC-Cy7 LIVE/DEAD™ Fixable Near-IR Dead Cell Stain Kit, cat #L10119) and with anti-CD8 conjugated to Alexa-Fluor 421 (Biolegend). The cells were analyzed both by flow-cytometry and by microscopy with a ImageStream®X Mk II Imaging Flow Cytometer (Luminex).

The effector cells and the target cells were distinguished by CD8 staining, as the p815 cells and K562 cells are negative for CD8 (FIG. 7B). When the CD8 negative cells were examined a large proportion of them were found to be GFP positive (FIG. 7C) indicating that the Cas9-GFP fusion protein was successfully transferred to the target cells. Cell imaging confirmed the presence of CD8−/GFP+ cells (FIG. 7D). The GFP signal in the target cells was disperse, indicating that protein reaches the cytoplasm. Similar results were found for transfer to p815 cells and K562 cells. The results are summarized in Table 6.

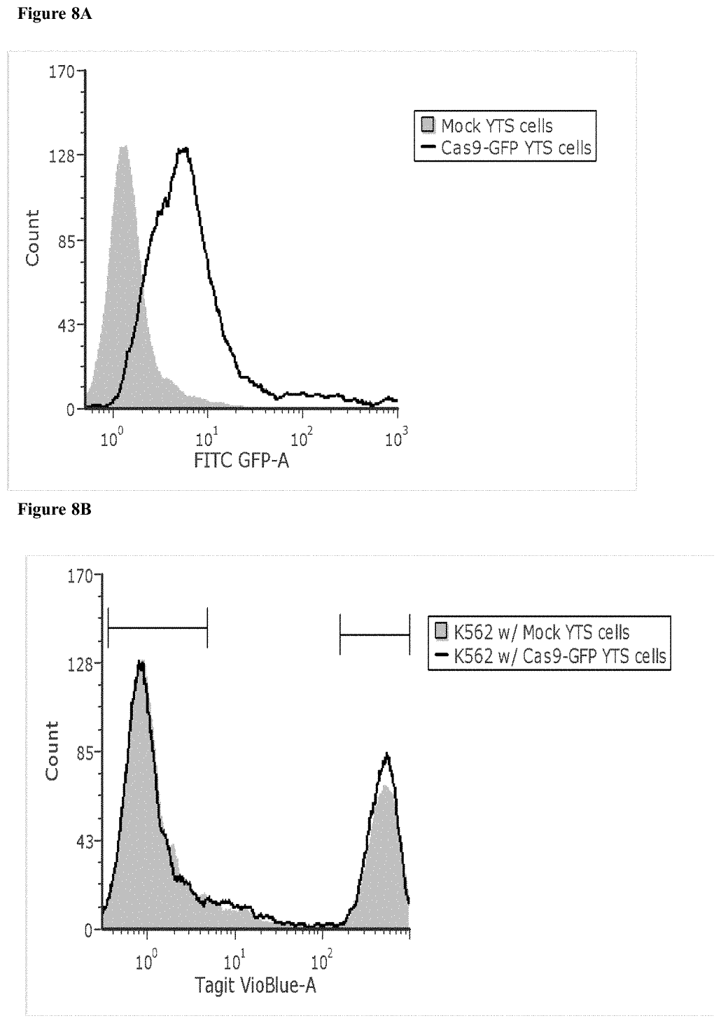

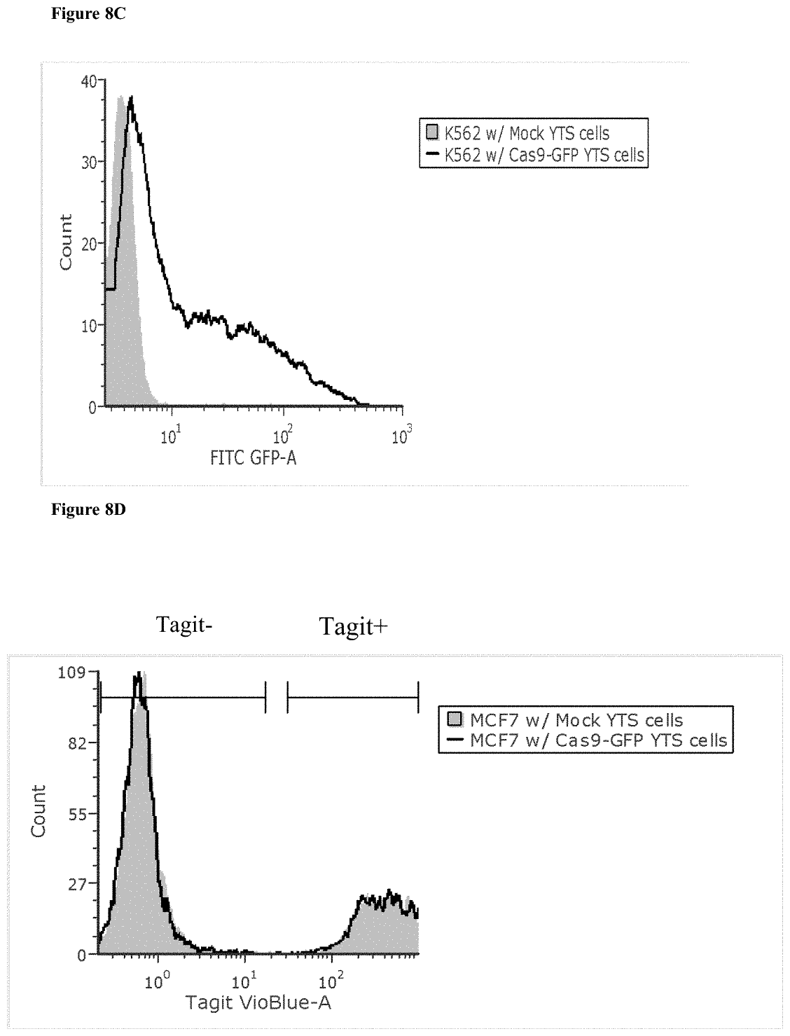

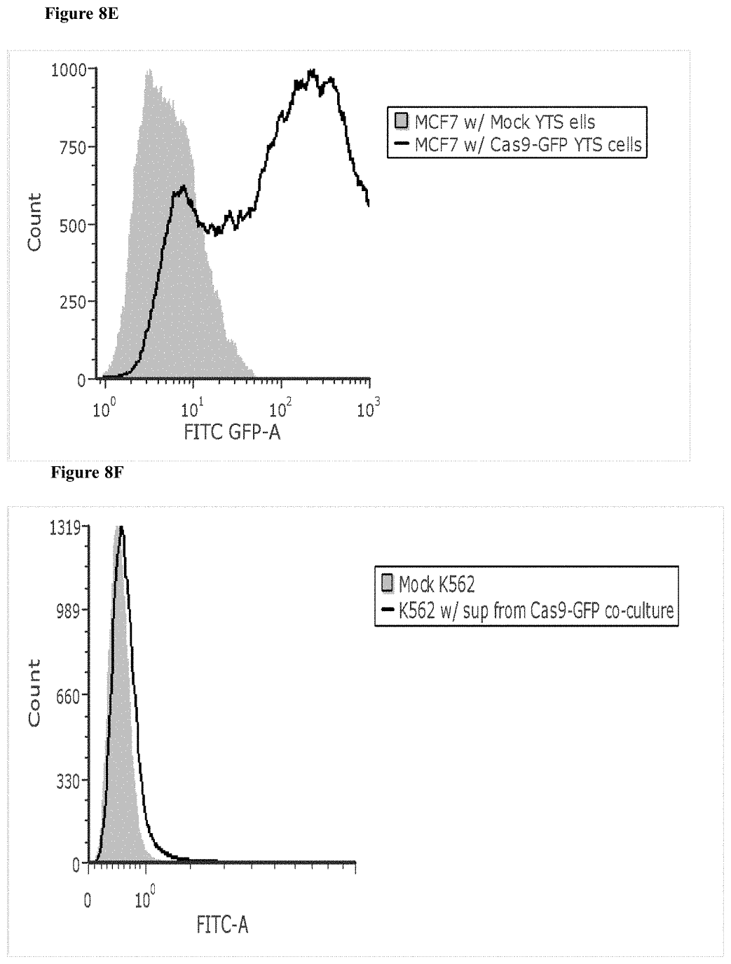

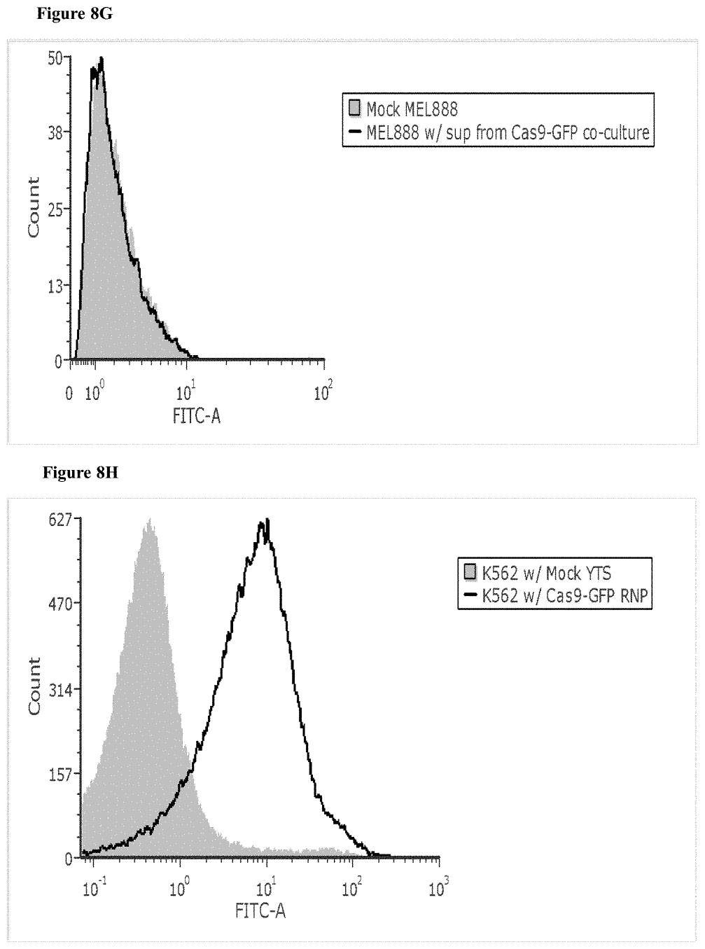

The same experiments were performed with YTS cells effector cells and K562 or human MCF7 breast cancer cells as target cells. The YTS cells also expressed high levels of GFP (>50% GFP positive) (FIG. 8A). The target cells were stained with Tag-it cell viability dye and could therefore be distinguished from the YTS cells due to their being Tag-it positive (FIGS. 8B and 8D). The Tag-it positive cells were then assessed for GFP expression. Both K562 (FIG. 8C) and MCF7 cells (FIG. 8E) were found to be highly GFP positive indicating that even the very large RNA-Protein complex was able to transfer and even without fusion to Granzyme B. The results of transfer are summarized in Table 6.

TABLE 6
Results of RNA-Protein complex transfer

| Effector Cells | Target Cells | Transfer results | Target cell viability |
|---|---|---|---|
| T cells | Mouse p815 | 40% | 50% |
| | (replicate 1) | | |
| T cells | Mouse p815 | 25% | 92% |
| | (replicate 2) | | |
| T cells | Human K562 | 65% | 80% |
| YTS cells | Human K562 | 56% | 65% |
| YTS cells | Human K562 | 37% | 80% |
| YTS cells | Human MCF7 | 77% | 47% |

Even though the RNP did not contain Granzyme protein, it was hypothesized that the transfer was still mediated by the lytic granule and synapse formation. This would mean that exosomes or other extracellular vesicles could not simply be isolated from the cells as direct contact and recognition of the target cells would be necessary for transfer. To test this hypothesis YTS cells were transduced with CAS9-GFP and cultured for 14-16 hours after which media from the cells was collected. The conditioned media was added to target K562 cells and melanoma cells and incubated overnight. Neither the K562 (FIG. 8F) nor the melanoma cells (FIG. 8G) were found to become GFP positive. When the effector cells were directly co-cultured with the target K562 cells (FIG. 8H) and melanoma cells (FIG. 8I) the target cells became highly GFP positive indicating protein transfer. This indicates that secreted vesicles, like exosomes, are not responsible for this non-Granzyme mediated transfer, but rather direct cellular contact is required.

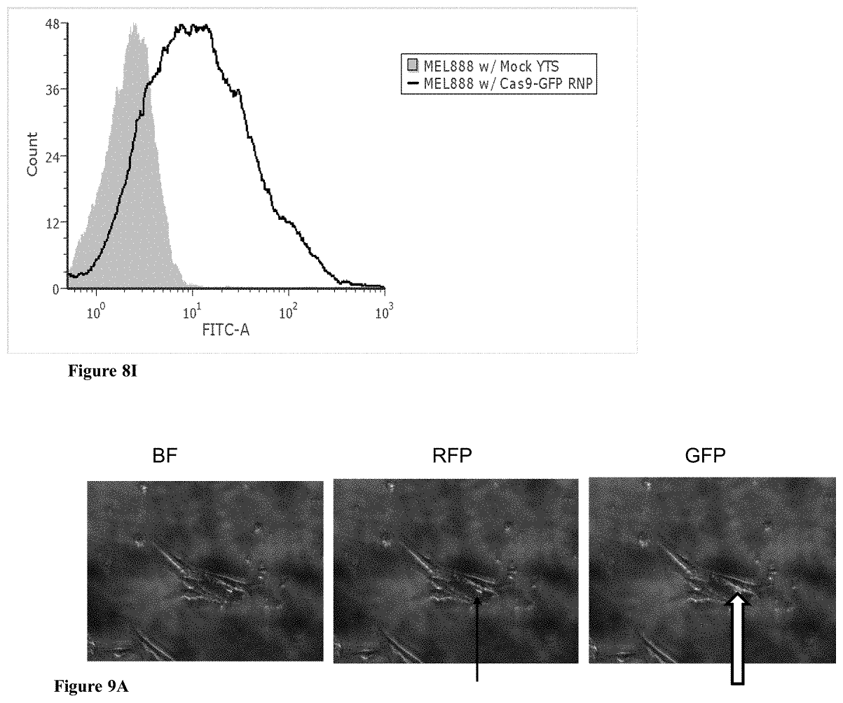

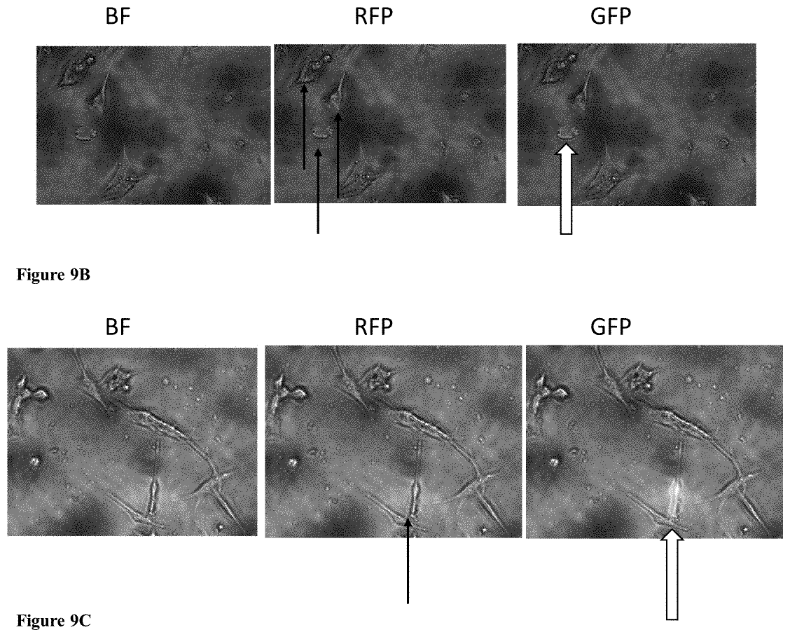

As it is now confirmed that CAS9-sgRNA complexes are transferred from effector to target cells, the ability of the RNA-Protein complex to carry out gene-editing in the target cells was also assessed. YTS and T cells were electroporated with RNA-Protein complexes as described hereinabove. The target K562 and melanoma cells were electroporated or transfected (respectively) with a PNA-reporter plasmid with an EMX1 sequence in the cleavage target area. Effector and target cells were co-cultured for 4 hours followed by sorting of target cells based on the RFP signal of the reporter plasmid. Sorted target cells were cultured for 3 days and editing was measured as before based on the presence of GFP signal in RFP gated cells. Even though the RNA-Protein complex did not comprise Granzyme, the CAS9 complex transferred, retained its sgRNA and was able to induce editing in the target cell (FIG. 9A). Similar results were observed for YTS cells and T cells used as the effector cells and for melanoma and K562 cells used as the target cells. Importantly, YTS cells that had endogenous Granzyme B knocked out and thus had reduced cytotoxicity, were also competent for transfer, sgRNA retention and editing in the target cell (FIG. 9B).

Next, it was examined if myeloid cells could also transfer RNA-protein complex that was capable of performing gene-editing in target cells. K562 cells were transduced with the CAS9-sgRNA complex as was done with the T cells and YTS cells. They were then co-cultured with melanoma cells expressing the editing reporter plasmid and it was observed that editing still occurred in the target cells (FIG. 9C). This indicates that myeloid cells, and indeed other perforin/granzyme expressing cells, are capable of transferring cargo and indeed are specifically capable of delivering functional genome-editing proteins/complexes.

Having shown editing of the exogenous PNA plasmid, editing of the endogenous genome by RNP delivery was tested. T cells, YTS cells, Granzyme B KO YTS cells and K562 cells were all used as effector cells to test RNP delivery and genome-editing. Melanoma cells, K562 cells and mouse P815 cells were all used as target cells. Editing events were quantified as described hereinabove by next-generation sequencing of the target locus. The results of the endogenous gene editing are summarized in Table 7. These results confirm that not only is the RNP transferred, but the guide RNA stays associated with the CAS9 protein during transfer and the complex is able to carry out genome-editing in the nucleus of the target cell.

TABLE 7
Results of endogenous gene editing after RNP
transfer normalized to viable cell result.

| Effector cell | Target cell | Editing |
|---|---|---|
| T Cell | MEL | 8.3% |
| YTS GZMB KO | K562 | 2.87% |
| YTS | MEL | 5% |
| YTS GZMB KO | MEL | 2.8% |
| K562 | MEL | 5.6% |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of delivering a non-lytic therapeutic protein of interest into a target cell, the method comprising contacting said target cell with a modified leukocyte wherein said modified leukocyte comprises reduced cytotoxicity as compared to a non-modified leukocyte and comprises said non-lytic therapeutic protein of interest, wherein said non-lytic therapeutic protein of interest is a chimeric protein comprising a lymphocyte lytic granule-secreted protein or a functional fragment or variant thereof and a therapeutic polypeptide, thereby delivering a non-lytic therapeutic protein of interest into a target cell.

2. (canceled)

3. The method of claim 1, wherein the therapeutic polypeptide is selected from:
a. delivered to the cytoplasm or nucleus of the target cell,
b. comprising a genome editing protein;
c. not comprising any of: a naturally secreted protein, a membranal protein expressed in the membrane of the modified leukocyte, a surface receptor-binding protein, a viral penetration or envelope protein and a nanoparticle conjugated or encapsulated protein;
d. a cytoplasmic or nuclear protein;
e. not comprising a signal peptide;
f. comprising a ribonuclear-protein (RNP) complex;
g. comprising a molecule weight of at least 50 kDa; and
h. any combination thereof.

4. The method of claim 1, wherein said modified leukocyte:
a. is capable of forming an immune synapse with said target cell;
b. comprises said non-lytic therapeutic protein of interest or genome editing protein within a secretory lysosome;
c. does not comprise said non-lytic therapeutic protein of interest or genome editing protein within or conjugated to said modified cell's cellular membrane;
d. is selected from a modified T cell, modified natural killer (NK) cell and a modified myeloid cell;
e. is a modified non-cytotoxic leukocyte, or wherein said modified leukocyte has been further modified to reduce cytotoxicity;
f. comprises a knockout or knockdown of at least one endogenous cytotoxic protein, optionally wherein said endogenous cytotoxic protein is Granzyme B;
g. comprises a mutation in at least one endogenous cytotoxic protein and wherein said mutation decreases cytotoxicity of said endogenous cytotoxic protein;
h. comprises an anti-sense mediated reduction of at least one endogenous cytotoxic protein and wherein said anti-sense mediated decreases cytotoxicity of said endogenous cytotoxic protein: or
i. any combination thereof.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. The method of claim 1, wherein said chimeric protein comprises a signal peptide, optionally wherein said signal peptide is an N-terminal signal peptide.

23. The method of claim 1, wherein said lymphocyte lytic granule-secreted protein is directly conjugated to said therapeutic polypeptide by a peptide bond or is indirectly conjugated by a protein linker.

24. The method of claim 23, wherein said linker is a cleavable linker, optionally wherein said linker is cleaved in a secretory granule or at acidic pH.

25. The method of claim 1, wherein said therapeutic polypeptide or genome editing protein comprises a nuclear localization sequence (NLS).

26. The method of claim 1, wherein said lytic granule-secreted protein is a lytic protein comprising at least one inactivating mutation, wherein said inactivating mutation inhibits the lytic function of said lytic protein.

27. The method of claim 1, wherein said lymphocyte lytic granule secreted protein is selected from the group consisting of: granzyme A, granzyme B, granzyme H, granzyme K, granzyme M, Granulysin, Serglycin, and Perforin.

28. (canceled)

29. (canceled)

30. (canceled)

31. The method of claim 1, further comprising providing said leukocyte, activating said leukocyte and expressing said non-lytic therapeutic protein of interest in said leukocyte after said activating to produce said modified leukocyte.

32. The method of claim 31, wherein said expression is done not more than 5 days before said contacting.

33. The method of claim 1, wherein said target cell is in a subject in need of treatment with said non-lytic therapeutic protein of interest, and said method comprises administering a pharmaceutical composition comprising said modified leukocyte.

34. (canceled)

35. (canceled)

36. The method of claim 33, comprising extracting leukocytes from said subject, activating said leukocytes, expressing said non-cytotoxic therapeutic protein of interest or genome editing protein in said leukocytes after said activating to produce said modified leukocytes and returning said modified leukocytes to said subject, optionally wherein said expressing is done not more than 5 days before said returning.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A modified leukocyte with reduced cytotoxicity as compared to a non-modified leukocyte, comprising at least one of:
a. a chimeric polypeptide comprising a lymphocyte lytic granule-secreted protein or a functional fragment or variant thereof and a therapeutic polypeptide; and
b. a polynucleotide encoding said chimeric polypeptide.

54. The modified leukocyte of claim 53, wherein said leukocyte
a. is capable of forming an immune synapse with a target cell;
b. is selected from a T cell, a natural killer (NK) cell, and a myeloid cell;
c. does not comprise said non-cytotoxic therapeutic protein of interest within or conjugated to said modified leukocyte's cellular membrane;
d. comprises a mutation of at least one endogenous cytotoxic protein wherein said mutation decreases the cytotoxicity of said endogenous cytotoxic protein: or
e. a combination thereof.

55. (canceled)

56. (canceled)

57. (canceled)

58. The modified leukocyte of claim 53, wherein said modified leukocyte comprises a knockout or knockdown or at least one endogenous cytotoxic protein, optionally wherein said endogenous cytotoxic protein is Granzyme B.

59. A therapeutic composition comprising a modified leukocyte of claim 53.

60. The therapeutic composition of claim 59, formulated for administration to a subject, and comprising a pharmaceutically acceptable carrier, excipient, or adjuvant or both.

61. A kit comprising
a modified leukocyte of claim 53.

62. The method of claim 1, wherein said modified leukocyte is a myeloid cell.


SEQUENCE LISTING (75 sequences)

SEQ ID NO: 1 - protein, 236 aa, Artificial
  Synthetic
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 2 - protein, 10 aa, Artificial
  Synthetic
VSKGEEDNMA

SEQ ID NO: 3 - protein, 225 aa, Artificial
  Synthetic
IIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 4 - protein, 11 aa, Artificial
  Synthetic
MVSKGEEDNMA

SEQ ID NO: 5 - DNA, 3916 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggcccccaagaagaagaggaaggtgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagcccctagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 6 - DNA, 3943 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggcccccaagaagaagaggaaggtgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagccccggatccccaaaaaagaaaagaaaagtttagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 7 - DNA, 678 bp, Artificial
  Synthetic
atcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 8 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 9 - protein, 10 aa, Artificial
  Synthetic
GSGSGSGSGS

SEQ ID NO: 10 - protein, 5 aa, Artificial
  Synthetic
GGGGS

SEQ ID NO: 11 - protein, 20 aa, Artificial
  Synthetic
GGGGSGGGGSGGGGSGGGGS

SEQ ID NO: 12 - protein, 9 aa, Artificial
  Synthetic
RARDPPVAT

SEQ ID NO: 13 - protein, 6 aa, Artificial
  Synthetic
  misc_feature (2)..(2): Xaa can be any naturally occurring amino acid
DXDPHF

SEQ ID NO: 14 - protein, 5 aa, Artificial
  Synthetic
GTGDP

SEQ ID NO: 15 - DNA, 888 bp, Homo sapiens
agctccaaccagggcagccttcctgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctcttagctgagtgga

SEQ ID NO: 16 - protein, 247 aa, Mus musculus
MKILLLLLTLSLASRTKAGEIIGGHEVKPHSRPYMALLSIKDQQPEAICGGFLIREDFVLTAAHCEGSIINVTLGAHNIKEQEKTQQVIPMVKCIPHPDYNPKTFSNDIMLLKLKSKAKRTRAVRPLNLPRRNVNVKPGDVCYVAGWGRMAPMGKYSNTLQEVELTVQKDRECESYFKNRYNKTNQICAGDPKTKRASFRGDSGGPLVCKKVAAGIVSYGYKDGSPPRAFTKVSSFLSWIKKTMKSS

SEQ ID NO: 17 - DNA, 741 bp, Mus musculus
atgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagc

SEQ ID NO: 18 - DNA, 740 bp, Artificial
  Synthetic
atgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgcta

SEQ ID NO: 19 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 20 - protein, 1368 aa, Streptococcus pyogenes [sequence omitted]

SEQ ID NO: 21 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1082 aa, Neisseria meningitidis
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 23 - protein, 1087 aa, Bacillus stearothermophilus
MRYKIGLDIGITSVGWAVINLDIPRIEDLGVRIFDRAENPQTGESLALPRRLARSARRRLRRRKHRLERIRRLIIREGILTKEELDKLFEEKHEIDVWQLRVEALDRKLNNDELARVLLHLAKRRGFKSNRKSERSNKENSTMLKHIEENRAILSGYRTVGEMIVKDPKFALHKRNKGENYTNTIARDDLEHEIRLIFSKQREFGNMSCTEKFENEYITIWASQRPVASKDDIEKKVGFCTFEPKEKRAPKATYTFQSFIAWEHINKLRLIFPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTSHLRSRWDFNKNREESDLHHAVDAAIVACTTPSDIAKVTAFYQRREQNKELAKKTEPHFPQPWPHFADELRARLSKHPKESIKALNLGNYDDQKLESLQPVFVSRMPKRSVTGAAHQETLRRYIGIDERSGKIQTVVKTKLSEIKLDASGHFPMYGKESDPRTYEAIRQRLLEHNNDPKKAFQEPLYKPKKNGEPGPIIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTTTGEEINVKDVFVYYKTIDSATGGLELISHDNRFSLRGVGSRTLKRFEKYQVDVLGNIYKVRGEKRVGLASSAHSKTGETIRPLQSTRD

SEQ ID NO: 24 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 25 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 26 - protein, 1228 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 27 - DNA, 6354 bp, Artificial
  Synthetic
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccctgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 28 - DNA, 6354 bp, Artificial
  Synthetic
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 29 - DNA, 4312 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 30 - DNA, 4312 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 31 - DNA, 8536 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 32 - DNA, 8536 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 33 - DNA, 8506 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 34 - DNA, 8506 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 35 - DNA, 4312 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgagggaggcgggggttctggcgggggtggatcagggggtggaggctccggtggaggcgggtcggctagcatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 36 - DNA, 4312 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcagcagcaatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgagggaggcgggggttctggcgggggtggatcagggggtggaggctccggtggaggcgggtcggctagcatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 37 - DNA, 8536 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 38 - DNA, 8536 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcagcagcaatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 39 - DNA, 8506 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 40 - DNA, 8506 bp, Artificial
  Sythetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgaagatcctcctgctactgctgaccttgtctctggcctccaggacaaaggcagcagcaatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagcctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 41 - DNA, 740 bp, Artificial
  Synthetic
atgaagatcctcctgctactgctgaccttgtctctggcctccaggacaaaggcagcagcaatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcag

SEQ ID NO: 42 - DNA, 4104 bp, Streptococcus pyogenes
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgat

SEQ ID NO: 43 - protein, 3 aa, Artificial
  Synthetic
GGS

SEQ ID NO: 44 - protein, 12 aa, Artificial
  Synthetic
GGSGGSGGSGGS

SEQ ID NO: 45 - DNA, 19 bp, Artificial
  Synthetic
ggagccaaguccagauuua

SEQ ID NO: 46 - DNA, 21 bp, Artificial
  Synthetic
uaaaucuggacuuggcuccuu

SEQ ID NO: 47 - DNA, 19 bp, Artificial
  Synthetic
cgcuguaaugaaacaccuu

SEQ ID NO: 48 - DNA, 21 bp, Artificial
  Synthetic
aagguguuucauuacagcguu

SEQ ID NO: 49 - DNA, 20 bp, Artificial
  Synthetic
gucaccuccaaugacuaggg

SEQ ID NO: 50 - DNA, 20 bp, Artificial
  Synthetic
acuccagucuuucuagaaga

SEQ ID NO: 51 - DNA, 20 bp, Artificial
  Synthetic
cugacagcugcucacuguug

SEQ ID NO: 52 - DNA, 20 bp, Artificial
  Synthetic
aagucucugaagaggugcgg

SEQ ID NO: 53 - protein, 356 aa, Artificial
  Synthetic
MHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 54 - DNA, 1068 bp, Artificial
  Synthetic
atgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagcccc

SEQ ID NO: 55 - DNA, 47 bp, Artificial
  Synthetic
acactgacgacatggttctacagttccagaaccggaggacaaagtac

SEQ ID NO: 56 - DNA, 40 bp, Artificial
  Synthetic
tacggtagcagagacttggtcttccaggcctccccaaagc

SEQ ID NO: 57 - DNA, 42 bp, Artificial
  Synthetic
acactgacgacatggttctacagaggcggatcacaagcaata

SEQ ID NO: 58 - DNA, 40 bp, Artificial
  Synthetic
tacggtagcagagacttggtctgggaggggagtgttgcaa

SEQ ID NO: 59 - DNA, 42 bp, Artificial
  Synthetic
acactgacgacatggttctacacctggctatggtagggacag

SEQ ID NO: 60 - DNA, 42 bp, Artificial
  Synthetic
tacggtagcagagacttggtctgcagtggggtggtgacttac

SEQ ID NO: 61 - DNA, 41 bp, Artificial
  Synthetic
acactgacgacatggttctacaccaactcctaagccagtgc

SEQ ID NO: 62 - DNA, 43 bp, Artificial
  Synthetic
tacggtagcagagacttggtctagtgcctatcagaaacccaag

SEQ ID NO: 63 - DNA, 42 bp, Artificial
  Synthetic
acactgacgacatggttctacaccacaacgaggactacacca

SEQ ID NO: 64 - DNA, 42 bp, Artificial
  Synthetic
tacggtagcagagacttggtcttggtgcagatgaacttcagg

SEQ ID NO: 65 - DNA, 8485 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 66 - DNA, 8485 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgagggaggcgggggttctggcgggggtggatcagggggtggaggctccggtggaggcgggtcggctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 67 - DNA, 8455 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 68 - DNA, 8455 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacctcgaggtgagcaagggcgaggaggataacatggccgctagcatgtacccatacgatgttccagattacgctggtaccatggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggatccccaaaaaagaaaagaaaagttgccaccaacttcagcctgctgaagcaggccggcgacgtggaggagaaccccggccccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 69 - DNA, 4720 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacagcggcttcgccaacgagcttggacccaggttgatgggaaagcccaagaagaagaggaaggtgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagccccggatccccaaaaaagaaaagaaaagtttagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 70 - DNA, 4672 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatggcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacagcggcttcgccaacgagcttggacccaggttgatgggaaagcccaagaagaagaggaaggtgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagccccggatccccaaaaaagaaaagaaaagtttagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 71 - DNA, 5437 bp, Artificial
  Synthetic
cgtctcgtcccggtctcctcccatgcatgtcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcaaccaatcctgcttctgctggccttcctcctgctgcccagggcagatgcagcagcaatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacagcggcttcgccaacgagcttggacccaggttgatgggaaagcccaagaagaagaggaaggtgcacatgaacaccaagtacaacaaggagttcctgctgtacctggccggcttcgtggacggcgacggcagcatcttcgccaggatcaagcccagccagaggagcaagttcaagcacaagctgcacctggtgttcgccgtgtaccagaagacccagaggaggtggttcctggacaagctggtggacgagatcggcgtgggctacgtgctggacagcggcagcgtgagcttctacagcctgagcgagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgcccggcagcgtgggcggcctgagccccagccaggccagcagcgccgccagcagcgccagcagcagccccggcagcggcatcagcgaggccctgagggccggcgccggcagcggcaccggctacaacaaggagttcctgctgtacctggccggcttcgtggacggcgacggcagcatctacgccaggatcaagcccgtgcagagggccaagttcaagcacgagctggtgctgggcttcgacgtgacccagaagacccagaggaggtggttcctggacaagctggtggacgagatcggcgtgggctacgtgtacgacaagggcagcgtgagcgcctacaggctgagccagatcaagcccctgcacaacttcctgacccagctgcagcccttcctgaagctgaagcagaagcaggccaacctggtgctgaagatcatcgagcagctgcccagcgccaaggagagccccgacaagttcctggaggtgtgcacctgggtggaccagatcgccgccctgaacgacagcaagaccaggaagaccaccagcgagaccgtgagggccgtgctggacagcctgagcgagaagaagaagagcagccccggaggtggaggttctggaggtggtggatcaggaggtggaggttctggtggaggtggatctcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccccaaaaaagaaaagaaaagtttagagatctcgagctcgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacttaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggtt

SEQ ID NO: 72 - protein, 14 aa, Artificial
  Synthetic
SGFANELGPRLMGK

SEQ ID NO: 73 - protein, 2117 aa, Artificial
  Synthetic
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 74 - protein, 2117 aa, Artificial
  Synthetic
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 75 - DNA, 23 bp, Artificial
  Synthetic
uugcacgagaucgccagcggcgg